SEQUENCE LISTING (346 sequences)

SEQ ID NO: 1 - DNA, 75 bp, Artificial Sequence
  Description of Artificial Sequence Synthetic oligonucleotide
gctcctgatgtacaggtaacccgtnnnnnnnnnnnnnnnnnnnnnnnngacnnntactatgcatacacggtgacc

SEQ ID NO: 2 - protein, 92 aa, Artificial Sequence
  Description of Artificial Sequence Synthetic consensus sequence
DINVQVNIDDNGKNFDYAYTVTTESELQRVLNELRDYIKKQGAKRVRISITARTKKEAEQFAAILIDVFADLGYNDIXVTXTGDTVRVEGQL

SEQ ID NO: 3 - protein, 42 aa, Artificial Sequence
  Description of Artificial Sequence Synthetic consensus sequence
INVQVYAYLQRVLNELRDYIISIFAAILIDVFIXVTXVRVEG

SEQ ID NO: 4 - DNA, 81 bp, Artificial Sequence
  Description of Artificial Sequence Synthetic oligonucleotide
ctgaacgagctcaaagactacattaaannnnnnnnnnnnnnnnnnnnnnnngttnnnatttctattaccgcgcgcactaaa

SEQ ID NO: 5 - protein, 92 aa, Artificial Sequence
  Description of Artificial Sequence Synthetic consensus sequence
DINVQVNIDDNGKNFDYAYTVTTESELQKVLNELKDYIKKQGAKRVRISITARTKKEAEQFAAILIKVFADLGYNDIXVTFTGDTVTVEGQL

SEQ ID NO: 6 - protein, 42 aa, Artificial Sequence
  Description of Artificial Sequence Synthetic consensus sequence
INVQVYAYLQKVLNELKDYIISIFAAILIKVFIXVTWVTVEG

SEQ ID NO: 7 - protein, 42 aa, Artificial Sequence
  Description of Artificial Sequence Synthetic consensus sequence
INVQVYAYLQXVLXELXDXIISXFAAILXXVFIXVTXVXVEG

SEQ ID NO: 8 - DNA, 56 bp, Artificial Sequence
  Description of Artificial Sequence Synthetic oligonucleotide
aagtattcgctgacctaggannnnnnnnnattaacgtcacttggaccggtgacaca

SEQ ID NO: 9 - DNA, 60 bp, Artificial Sequence
  Description of Artificial Sequence Synthetic oligonucleotide
acttggaccggtgacacagtaacagtagaaggannnnnntaataactcgaggaagcttgg

SEQ ID NO: 10 - DNA, 24 bp, Artificial Sequence
  Description of Artificial Sequence Synthetic primer
gctcctgatgtacaggtaacccgt

SEQ ID NO: 11 - DNA, 21 bp, Artificial Sequence
  Description of Artificial Sequence Synthetic primer
ggtcaccgtgtatgcatagta

SEQ ID NO: 12 - DNA, 27 bp, Artificial Sequence
  Description of Artificial Sequence Synthetic primer
ctgaacgagctcaaagactacattaaa

SEQ ID NO: 13 - DNA, 24 bp, Artificial Sequence
  Description of Artificial Sequence Synthetic primer
tttagtgcgcgcggtaatagaaat

SEQ ID NO: 14 - DNA, 20 bp, Artificial Sequence
  Description of Artificial Sequence Synthetic primer
aagtattcgctgacctagga

SEQ ID NO: 15 - DNA, 27 bp, Artificial Sequence
  Description of Artificial Sequence Synthetic primer
tgtgtcaccggtccaagtgacgttaat

SEQ ID NO: 16 - DNA, 33 bp, Artificial Sequence
  Description of Artificial Sequence Synthetic primer
acttggaccggtgacacagtaacagtagaagga

SEQ ID NO: 17 - DNA, 21 bp, Artificial Sequence
  Description of Artificial Sequence Synthetic primer
ccaagcttcctcgagttatta

SEQ ID NO: 18 - protein, 12 aa, Artificial Sequence
  Description of Artificial Sequence Synthetic peptide
EGGSLEHHHHHH

SEQ ID NO: 19 - protein, 106 aa, Artificial Sequence
  Description of Artificial Sequence Synthetic polypeptide
XGDIQVQVNIDDNGKNFDYTYTVTTESELQKVLNELXDYIKKQGAKRVRISITARTKKEAEKFAAILIKVFAELGYNDINVTFDGDTVTVEGQLEGGSLEHHHHHH

SEQ ID NO: 20 - protein, 92 aa, Artificial Sequence
  Description of Artificial Sequence Synthetic polypeptide
DIQVQVNIDDNGKNFDYTYTVTTESELQKVLNELKDYIKKQGAKRVRISITARTKKEAEKFAAILIKVFAELGYNDINVTFDGDTVTVEGQL

SEQ ID NO: 21 - protein, 92 aa, Artificial Sequence
  Description of Artificial Sequence Synthetic polypeptide
DINVQVNIDDNGKNFDYAYTVTTESELQKVLNELKDYIKKQGAKRVRISITARTKKEAEQFAAILIKVFADLGYNDINVTWTGDTVTVEGQL

SEQ ID NO: 22 - protein, 97 aa, Artificial Sequence
  Description of Artificial Sequence Synthetic polypeptide
DINVQVTRDXXXXXXXXDXYAYTVTTESELQKVLNELKDYIKXXXXXXXXVXISITARTKKEAEQFAAILIKVFADLGXXXINVTWTGDTVTVEGXX

SEQ ID NO: 23 - protein, 93 aa, Artificial Sequence
  Description of Artificial Sequence Synthetic polypeptide
DIQVQVNIDDNGKNFDYTYTVTTESELQKVLNELKDYIKKQGAKRVRISITARTKKEAEKFAAILIKVFAELGYNDINVTFDGDTVTVEGQLE

SEQ ID NO: 24 - protein, 100 aa, Artificial Sequence
  Description of Artificial Sequence Synthetic polypeptide
MADINVQVNIDDGGGGGGGGKNFDYAYTVTTESELQKVLNELKDYIKKQGAKRVRISITARTKKEAEQFAAILIKVFADLGYNDINVTWTGDTVTVEGQL

SEQ ID NO: 25 - protein, 97 aa, Artificial Sequence
  Description of Artificial Sequence Synthetic polypeptide
MADINVQVNIDDNGKNFDYAYTVTTESELQKVLNELKDYIKGGGGGGGGRVRISITARTKKEAEQFAAILIKVFADLGYNDINVTWTGDTVTVEGQL

SEQ ID NO: 26 - protein, 99 aa, Artificial Sequence
  Description of Artificial Sequence Synthetic polypeptide
MADINVQVNIDDNGKNFDYAYTVTTESELQKVLNELKDYIKKQGAKRVRISITARTKKEAEQFAAILIKVFADLGGGGGGGGDINVTWTGDTVTVEGQL

SEQ ID NO: 27 - protein, 108 aa, Artificial Sequence
  Description of Artificial Sequence Synthetic polypeptide
MADINVQVNIDDGGGGGGGGKNFDYAYTVTTESELQKVLNELKDYIKGGGGGGGGRVRISITARTKKEAEQFAAILIKVFADLGGGGGGGGDINVTWTGDTVTVEGQL

SEQ ID NO: 28 - protein, 110 aa, Artificial Sequence
  Description of Artificial Sequence Synthetic polypeptide
MADINVQVNIDDNGKNFDYAYTVTTESELQKVLNELKDYIKKSGKETAAAKFERQHMDSGGKRVRISITARTKKEAEQFAAILIKVFADLGYNDINVTWTGDTVTVEGQL

SEQ ID NO: 29 - protein, 116 aa, Artificial Sequence
  Description of Artificial Sequence Synthetic polypeptide
MADINVQVNIDDGGGGGGGGKNFDYAYTVTTESELQKVLNELKDYIKKQGAKRVRISITARTKKEAEQFAAILIKVFADLGGKETAAAKFERQHMDSGNAINVTWTGDTVTVEGQL

SEQ ID NO: 30 - protein, 92 aa, Artificial Sequence
  Description of Artificial Sequence Synthetic polypeptide
DINVQVNIDDNGKNFDYAYTVTTESELQKVLNELKDYIKKQGAKRVRISITARTKKEAEQFAAILIKVFADLGYNDIDVTFTGDTVTVEGQL

SEQ ID NO: 31 - protein, 92 aa, Artificial Sequence
  Description of Artificial Sequence Synthetic polypeptide
DINVQVNIDDNGRNFDYAYRVTTEDELQRVLNELRDYIDRQGARRVRISIDAYTRREAERFAAILIDVFDDLGYNDINVTFTGDTVRVEGQL

SEQ ID NO: 32 - protein, 92 aa, Artificial Sequence
  Description of Artificial Sequence Synthetic polypeptide
DINVQVNIDDNGKNFDYAYRVTTEDELQKVLNELKDYIKKQGAKRVRISIDARTKKEAERFAAILIDVFDDLGYNDINVTFTGDTVRVEGQL

SEQ ID NO: 33 - protein, 92 aa, Artificial Sequence
  Description of Artificial Sequence Synthetic polypeptide
DINVQVNIDDNGKNFDYAYTVDTESELQKVLNELKDQIKKQGAKRVRISIDARTKKEAEQFAAILQKVFADLGYNDIDVTWTGDTVTVEGQL

SEQ ID NO: 34 - protein, 92 aa, Artificial Sequence
  Description of Artificial Sequence Synthetic polypeptide
DINVQVNIDDNGRNFDYAYTVDTESELQRVLNELADQIQRQGANRVRISIDARTQREAEQFAAILQQVFADLGYNDIDVTWTGDTVTVEGQL

SEQ ID NO: 35 - protein, 92 aa, Artificial Sequence
  Description of Artificial Sequence Synthetic polypeptide
DINVQVNIDDNGRNFDYAYTVDTESELQRVLDELADQIQRQGADRVRISVDARTQDEAEQFAAILQQVFADLGYNDIDVTWTGDTVTVEGQL

SEQ ID NO: 36 - protein, 91 aa, Artificial Sequence
  Description of Artificial Sequence Synthetic polypeptide
VQVTRDSAYRSPRDSYAYTVTTESELQKVLNELKDYIKKQGAKRVRISITARTKREAEQFAAILIKVFADLGPYSINVTWTGDTVTVEGSS

SEQ ID NO: 37 - protein, 93 aa, Artificial Sequence
  Description of Artificial Sequence Synthetic polypeptide
VQVTRDSAYRSPRDSYAYTVTTESELQKVLNELKDYIKKQGAKRVRISITARTKKEAEQFAAILIKVFADLGPYSINVTWTGDTVTVEGYLKR

SEQ ID NO: 38 - protein, 91 aa, Artificial Sequence
  Description of Artificial Sequence Synthetic polypeptide
VQVTRDSAYRSPRDSYAYTVTTESELQKVLNELKDYIKKQGAKRVRISITARTKKEAEQFAAILIKVFADLGPYSINVTWTGDTVTVEGSS

SEQ ID NO: 39 - protein, 91 aa, Artificial Sequence
  Description of Artificial Sequence Synthetic polypeptide
VQVTRDSAYRSPRDSYAYTVTTESELQKVLNELKDYIKKQGAKRVRISITARTKKEAEQFAAILIKVFADLGASYINVTWTGDTVTVEGSW

SEQ ID NO: 40 - protein, 91 aa, Artificial Sequence
  Description of Artificial Sequence Synthetic polypeptide
VQVTRDSAYRSPRDSYAYTVTTESELQKVLNELKDYIKKQGAKRVRISITARTKKEAEQFAAILIKVFADLGYIRINVTWTGDTVTVEGSP

SEQ ID NO: 41 - protein, 91 aa, Artificial Sequence
  Description of Artificial Sequence Synthetic polypeptide
VQVTRDSAYRSPRDSYAYTVTTESELQKVLNELKDYIKKQGAKRVRISITARTKKEAEQFAAILIKVFADLGYSSINVTWTGDTVTVEGLL

SEQ ID NO: 42 - protein, 91 aa, Artificial Sequence
  Description of Artificial Sequence Synthetic polypeptide
VQVTRDSAYRSPRDSYAYTVTTESELQKALNELKDYIKKQGAKRVRISITARTKKEAEQFAAILIKVFADLGYDDINVTWTGDTVTVEGLW

SEQ ID NO: 43 - protein, 92 aa, Artificial Sequence
  Description of Artificial Sequence Synthetic polypeptide
VQVTRDSAYRSPRDPYAYTVTTESELQKVLNELKDYIKYNNYGYYSVAISITARTKKEAEQFAAILIKVFADLGYAINVTWTGDTVTVEGRW

SEQ ID NO: 44 - protein, 93 aa, Artificial Sequence
  Description of Artificial Sequence Synthetic polypeptide
VQVTRDSAYRSPRDSYAYTVTTESELQKVLNELKDYIKWSYYAYLNVNISITARTKKEAEQFAAILIKVFADLGYYPINVTWTGDTVTVEGLA

SEQ ID NO: 45 - protein, 93 aa, Artificial Sequence
  Description of Artificial Sequence Synthetic polypeptide
VQVTRRSGYNYSSDYYAYTVTTESELQKVLNELKDYIKLLPYPRNIVYISITARTKKEAEQFAAILIKVFADLGAYDINVTWTGDTVTVEGYL

SEQ ID NO: 46 - protein, 92 aa, Artificial Sequence
  Description of Artificial Sequence Synthetic polypeptide
VQVTRRSGYNYSSDYYAYTVTTESLQKVLNKLKDYIKLLPYPRNIVYISITARTKKEAEQFAAILIKVFSDLRAYDINVTWTGDTVTVEGYL

SEQ ID NO: 47 - protein, 93 aa, Artificial Sequence
  Description of Artificial Sequence Synthetic polypeptide
VQVTRRSGYNYSSDYYAYTVTTESELRKVLNELKDYIKLLPYPRNIVYISITARTKKEAEQFAAILIKVFADLGPYSINVTWTGDTVTVEGSS

SEQ ID NO: 48 - protein, 94 aa, Artificial Sequence
  Description of Artificial Sequence Synthetic polypeptide
VQVTRYSAYNYSDDNYYAYTVTTESELQKVLNELKDYIKWNPPLYRAVAISITARTKKEAEQFAAILIKVFADLGAYDINVTWTGDTVTVEGYL

SEQ ID NO: 49 - protein, 95 aa, Artificial Sequence
  Description of Artificial Sequence Synthetic polypeptide
VQVTRYSAYNYSDDNYYAYTVTTESELQKVLNELKDYIKWNPPLYRAVAISITARTKKEAEQFAAILIKVFADLGAYDINVTWTGDTVTVEGKKI

SEQ ID NO: 50 - protein, 93 aa, Artificial Sequence
  Description of Artificial Sequence Synthetic polypeptide
VQVTRASAYSDYSDYYYAYTVTTESELQKVLNELKDYIKLSGWRNPPVYISITARTKKEAEQFAAILIKVFADLGYYNINVTTGDTVTVEGNS

SEQ ID NO: 51 - protein, 95 aa, Artificial Sequence
  Description of Artificial Sequence Synthetic polypeptide
VRVTRSNAYNRRADYYYAYTVTTESELQKVLNELKDYIKSSNYSNGRVIISITARTKKEAEQFAAILIKVFADLGSYAINVTWTGDTVTVEGWSS

SEQ ID NO: 52 - protein, 94 aa, Artificial Sequence
  Description of Artificial Sequence Synthetic polypeptide
VQVTRSNAYNRRADYYYAYTVTTESELQKVLNELKDYIKSSNYSNGRVIISITARTKKEAEQFAAILIKVFADLGSYAINVTWTGDTVTVEGWW

SEQ ID NO: 53 - protein, 94 aa, Artificial Sequence
  Description of Artificial Sequence Synthetic polypeptide
VQVTRALALYYNLDLYYAYTVTTESELQKVLNELKDYIKGSGYYNANVIISITARTKKEAEQFAAILIKVFADLGRYAINVTWTGDTVTVEGSS

SEQ ID NO: 54 - protein, 94 aa, Artificial Sequence
  Description of Artificial Sequence Synthetic polypeptide
VQVTRSYNDYPRADSYYAYTVTTESELQKVLNELKDYIKYGRSYPLAVRISITARTKKEAEQFAAILIKVFADLGLPPINVTWTGDTVTVEGNL

SEQ ID NO: 55 - protein, 96 aa, Artificial Sequence
  Description of Artificial Sequence Synthetic polypeptide
VQVTRSYNDYPRADSYYAYTVTTESELQKVLNELKDYIKYGRSYPLAVRISITARTKKEAEQFAAILIKVFADLGLPPINVTWTGDTVTVEGPSHN

SEQ ID NO: 56 - protein, 93 aa, Artificial Sequence
  Description of Artificial Sequence Synthetic polypeptide
VQVTRSYSNPADDYYYAYTVTTESELQKVLNELKDYIKSSGYYNLPVRISITARTKKEAEQFAAILIKVFADLGGNSINVTWTGDTVTVEGAA

SEQ ID NO: 57 - protein, 93 aa, Artificial Sequence
  Description of Artificial Sequence Synthetic polypeptide
VQVTRSYSNPADDYYYAYTVTTESELQKVLNELKDYIKSSGYYNLPVRISITARTKKEAEQFAAILIKVFADLGYAYINVTWTGDTVTVEGDP

SEQ ID NO: 58 - protein, 94 aa, Artificial Sequence
  Description of Artificial Sequence Synthetic polypeptide
VQVTRDYRAYPYWDGYYAYTVTTESELQKVLTELKDYIKYSNYYPLSVLISITARTKKEAEQFAAILIKVFADLGNPNINVTWTGDTVTVEGLW

SEQ ID NO: 59 - protein, 94 aa, Artificial Sequence
  Description of Artificial Sequence Synthetic polypeptide
VQVTRDSAWSAYYDGYYAYTVTTESELQKVLNELKDYIKYSNYYPLSVRISITARTKKEAEQFAAILIKVFADLGRWNINVTWTGDTVTVEGIS

SEQ ID NO: 60 - protein, 93 aa, Artificial Sequence
  Description of Artificial Sequence Synthetic polypeptide
VQVTRRSGYNYSSDYYAYTVTTESELQKVLNELKDYIKYSLSYPLYVYISITARTKKEAEQFAAILIKVFADLGYPAINVTWTGDTVTVEGAY

SEQ ID NO: 61 - protein, 93 aa, Artificial Sequence
  Description of Artificial Sequence Synthetic polypeptide
VQVTRNWDPYYSADYYAYTVTTESELQKVLNELKDYIKYTRSYPAYVIISITARTKKEAEQFAAILIKVFADLGLININVTWTGDTVTVEGAW

SEQ ID NO: 62 - protein, 94 aa, Artificial Sequence
  Description of Artificial Sequence Synthetic polypeptide
VQVTRPLSYASPYDAYYAYTVTTESELQKVLNELKDYIKYTRSYPAYVIISITARTKKEAEQFAAILIKVFADLGLININVTWTGDTVTVEGAW

SEQ ID NO: 63 - protein, 94 aa, Artificial Sequence
  Description of Artificial Sequence Synthetic polypeptide
VQITRGADYSRAGDYYYAYTVTTESELQKVLNELKDYIKSGWGYDNSVYISITARTKKEAEQFAAILIKVFADLGRLWINVTWTGDTVTVEGSA

SEQ ID NO: 64 - protein, 95 aa, Artificial Sequence
  Description of Artificial Sequence Synthetic polypeptide
VQITRGADYSRAGDYYYAYTVTTESELQKVLNELKDYIKYRDLSYNYVLISITARTKKEAEQFAAILIKVFADLGLYNINVTWTGDTVTVEGQLV

SEQ ID NO: 65 - protein, 94 aa, Artificial Sequence
  Description of Artificial Sequence Synthetic polypeptide
VQVTRPGYDSSIGDYYYAYTVTTESELQKVLNELKDYIKSRPSYLSYVYISITARTKKEAEQFAAILIKVFADLGDAYINVTWTGDTVTVEGPA

SEQ ID NO: 66 - protein, 92 aa, Artificial Sequence
  Description of Artificial Sequence Synthetic polypeptide
VQVTRDPRRPGGDYYAYTVTTESELQKVLNELKDYIKPSSILPYYVSISITARTKKEAEQFAAILIKVFADLGLININVTWTGDTVTVEGAW

SEQ ID NO: 67 - protein, 92 aa, Artificial Sequence
  Description of Artificial Sequence Synthetic polypeptide
VQVTRDPRRPGGDYYAYTVTTESELQKVLNELKDYIKPSSILPYYVSISITARTKKEAEQFAAILIKVFADLGLININVTWTGETVTVEGAW

SEQ ID NO: 68 - protein, 92 aa, Artificial Sequence
  Description of Artificial Sequence Synthetic polypeptide
VQVTRYPRRPGGDYYAYTVTTESELQKVLNELKDYIKPSSILPYYVSISITARTKKEAEQFAAILIKVFADLGLININVTWTGDTVTVEGAW

SEQ ID NO: 69 - protein, 92 aa, Artificial Sequence
  Description of Artificial Sequence Synthetic polypeptide
VQVTRDPRRPGGDYYAYTVTTESELQKVLNELKDYIKPSSILPYYVSISITARTKKEAEQFAAILIKVFADLGLASINVTWTGDTVTVEGYL

SEQ ID NO: 70 - protein, 92 aa, Artificial Sequence
  Description of Artificial Sequence Synthetic polypeptide
VQVTRDSLDNADSYYAYTVTTESELQKVLNELKDYIKNALDPRRRVYISITARTKKEAEQFAAILIKVFADLGLYWINVTWTGDTVTVEGYY

SEQ ID NO: 71 - protein, 91 aa, Artificial Sequence
  Description of Artificial Sequence Synthetic polypeptide
VQVNIDDNGKNFDYAYTVTTESELQKVLNELKDYIKLDSNYRYNVYISITARTKKEAEQFAAILIKVFADLGYAYINVTWTGDTVTVEGDP

SEQ ID NO: 72 - protein, 94 aa, Artificial Sequence
  Description of Artificial Sequence Synthetic polypeptide
VQVTRRPASASADDLYYAYTVTTESELQKVLNELKDYIKSRDYPIYYVIISITARTKKEAEQFAAILIKVFADLGSSSINVTWTGDTVTVEGEK

SEQ ID NO: 73 - protein, 94 aa, Artificial Sequence
  Description of Artificial Sequence Synthetic polypeptide
VQVTRPPNSASADDLYYAYPGTTESQLQKVLNELQDYIKWSYYSYLNVNISITARTKKEAEQFAAILIKVFPELRSSPINVTWTGDTVTVKGSP

SEQ ID NO: 74 - protein, 94 aa, Artificial Sequence
  Description of Artificial Sequence Synthetic polypeptide
VQVTRRRNSASADDLYYAYTVTTESELQKVLNELKDYIKYSNDYPANVSISITARTKKEAEQFAAILIKVFADLGWYSINVTWTGDTVTVEGYP

SEQ ID NO: 75 - protein, 94 aa, Artificial Sequence
  Description of Artificial Sequence Synthetic polypeptide
VQVTRIPDSASSADLYYAYTVTTESELQKVLNELKDYIKLDDRSDNYVSISITARTKKEAEQFAAILIKVFADLGYSYINVTWTGDTVTVEGYP

SEQ ID NO: 76 - protein, 97 aa, Artificial Sequence
  Description of Artificial Sequence Synthetic polypeptide
VQVTRYPWRLDRADYYYAYTVTTESELQKVLNELKDYIKWAYDNRNAVRISITARTKKEAEQFAAILIKVFADLGYNDINVTWTGDTVTVEGYRENN

SEQ ID NO: 77 - protein, 94 aa, Artificial Sequence
  Description of Artificial Sequence Synthetic polypeptide
VQVTRYPWRLDRADYYYAYTVTTESELQKVLNELKDYIKWAYDNRNAVRISITARTKKEAEQFAAILIKVFADLGYNDINVTWTGDTVTVEGYR

SEQ ID NO: 78 - protein, 94 aa, Artificial Sequence
  Description of Artificial Sequence Synthetic polypeptide
VQVTRSGYPWRPADDYYAYTVTTESELQKVLNELKDYIKNAYIAGNAVLISITARTKKEAEQFAAILIKVFADLGDSAINVTWTGDTVTVEGYY

SEQ ID NO: 79 - protein, 94 aa, Artificial Sequence
  Description of Artificial Sequence Synthetic polypeptide
VQVTRPSARSLLDDRYYAYTVTTESELQKVLNELKDYIKNWYYSSYHVLISITARTKKEAEQFAAILIKVFADLGCAYINVTWTGDTVTVEGEE

SEQ ID NO: 80 - protein, 94 aa, Artificial Sequence
  Description of Artificial Sequence Synthetic polypeptide
VQVTRRSARSLLDDRYYAYTVTTESELQKVLNELKDYIKNWYYSSYNVLISITARTKKEAEQFAAILIKVFADLGYAYINVTWTGDTVTVEGDP

SEQ ID NO: 81 - protein, 94 aa, Artificial Sequence
  Description of Artificial Sequence Synthetic polypeptide
VQVTRRSARSLLDDRYYAYTVTTESELQKVLNELKDYIKNWYYSSYNVLISITARTKKEAEQFAAILIKVFADLGGIPINVTWTGDTVTVEGLD

SEQ ID NO: 82 - protein, 94 aa, Artificial Sequence
  Description of Artificial Sequence Synthetic polypeptide
VQVTRAWLWPPNLDSYYAYTVTTESEQQKVLNELKDYIKKQGAKRVRISITARTKKEAEQFAAILIKVFADLGPYSINVTWTGDTVTVEGYYLE

SEQ ID NO: 83 - protein, 94 aa, Artificial Sequence
  Description of Artificial Sequence Synthetic polypeptide
VQVTRGASYPSNIDIYYAYTVTTESELQKVLNELKDYIKALYSSGYIVNISITARTKKEAEQFAAILIKVFADLGPWRINVTWTGDTVTVEGSN

SEQ ID NO: 84 - protein, 94 aa, Artificial Sequence
  Description of Artificial Sequence Synthetic polypeptide
VQVTRGLLYAGYSDSYYAYTVTTESELQKVLNELKDYIKPCYPYSWAVYISITARTKKEAEQFAAILIKVFADLGYRRINVTWTGDTVTVEGRL

SEQ ID NO: 85 - protein, 94 aa, Artificial Sequence
  Description of Artificial Sequence Synthetic polypeptide
VQVTRASASAPRYDNYYAYTVTTESELQKVLNELKDYIKLLPYPRNIVYISITARTKKEAEQFAAILIKVFADLGAYDINVTWTGDTVTVEGYL

SEQ ID NO: 86 - protein, 94 aa, Artificial Sequence
  Description of Artificial Sequence Synthetic polypeptide
VQVTRAWLWPPNLDSYYAYTVTTESEQHKVLNELKDYIKKQGAKRVRISITARTKKQAEQFAAILIKVFADLRTYSINVSWTGDPVTVKGYYLE

SEQ ID NO: 87 - protein, 96 aa, Artificial Sequence
  Description of Artificial Sequence Synthetic polypeptide
VQVTRLLYDGNSSDYYYAYTVTTESELQKVLNELKDYIKSDRGAAARVSISITARTKKEAEQFAAILIKVFADLGLSSINVTWTGDTVTVEGYLLE

SEQ ID NO: 88 - protein, 94 aa, Artificial Sequence
  Description of Artificial Sequence Synthetic polypeptide
VQVTRLLYDGNSSDYYYAYTVTTESELQKVLNELKDYIKSDRGAAARVSISITARTKKEAEQFAAILIKVFADLGLSSINVTWTGDTVTVEGYL

SEQ ID NO: 89 - protein, 94 aa, Artificial Sequence
  Description of Artificial Sequence Synthetic polypeptide
VQVTRLLYDGNSSDYYYAYTVTTESELQKVLNELKDYIKGYWGASARVDISITARTKKEAEQFAAILIKVFADLGLSSINVTWTGDTVTVEGYL

SEQ ID NO: 90 - protein, 95 aa, Artificial Sequence
  Description of Artificial Sequence Synthetic polypeptide
VQVTRLDYRSDNDYYYAYTVTTESELQKVLNELKDYIKNLAPGASRVNISITARTKKEAEQFAAILIKVFADLGLSSINVTWTGDTVTVEGYLLE

SEQ ID NO: 91 - protein, 94 aa, Artificial Sequence
  Description of Artificial Sequence Synthetic polypeptide
VQVTRLYYNGARSDYYYAYTVTTESELQKVLNELKDYIKYPSLYSYRVNISITARTKKEAEQFAAILIKVFADLGYASINVTWTGDTVTVEGYS

SEQ ID NO: 92 - protein, 96 aa, Artificial Sequence
  Description of Artificial Sequence Synthetic polypeptide
VQVTRLNAASSLGDRYYAYTVTTESELQKVLNELKDYIKSYYSYSSRVNISITARTKKEAEQFAAILIKVFADLGLSSINVTWTGDTVTVEGYLLE

SEQ ID NO: 93 - protein, 93 aa, Artificial Sequence
  Description of Artificial Sequence Synthetic polypeptide
VQVTRLAGARSSSDYYYAYTVTTESELQKVLNELKDYIKSASAPYRVDISITARTKKEAEQFAAILIKVFADLGLSSINVTWTGDTVTVEGSS

SEQ ID NO: 94 - protein, 97 aa, Artificial Sequence
  Description of Artificial Sequence Synthetic polypeptide
VQVTRSYPGASSSDAYYAYTVTTESELQKVLNELKDYIKPANLYSGRVLISITARTKKEAEQFAAILIKVFADLGLSSINVTWTGDTVTVEGNLLEE

SEQ ID NO: 95 - protein, 94 aa, Artificial Sequence
  Description of Artificial Sequence Synthetic polypeptide
VQVTRSYPGASSSDAYYAYTVTTESELQKVLNELKDYIKPANLYSGRVLISITARTKKEAEQFAAILIKVFADLGLASINVTWTGDTVTVEGGL

SEQ ID NO: 96 - protein, 97 aa, Artificial Sequence
  Description of Artificial Sequence Synthetic polypeptide
VQVTRSYPGASSSDAYYAYTVTTESELQKVLNELKDYIKPANLYSGRVLISITARTKKEAEQFAAILIKVFADLGLSSINVTWTGDTVTVEGYALEE

SEQ ID NO: 97 - protein, 97 aa, Artificial Sequence
  Description of Artificial Sequence Synthetic polypeptide
VQVTRSYPGASSSDAYYAYTVTTESELQKVLNELKDYIKPANLYSGRVLISITARTKKEAEQFAAILIKVFADLGYNDINVTWTGDTVTVEGQLVLE

SEQ ID NO: 98 - protein, 94 aa, Artificial Sequence
  Description of Artificial Sequence Synthetic polypeptide
VQVTRLYPLYSDSDNYYAYTVTTESELQKVLNELKDYIKWNINPSYRVLISITARTKKEAEQFAAILIKVFADLGLSSINVTWTGDTVTVEGSL

SEQ ID NO: 99 - protein, 97 aa, Artificial Sequence
  Description of Artificial Sequence Synthetic polypeptide
VQVTRLYPLSSPSDYYYAYTVTTESELQKVLNELKDYIKAPNYNYLSVYISITARTKKEAEQFAAILIKVFADLGLDYINVTWTGDTVTVEGYYLEE

SEQ ID NO: 100 - protein, 94 aa, Artificial Sequence
  Description of Artificial Sequence Synthetic polypeptide
VQVTRLYPLSSPSDYYYAYTVTTESELQKVLNELKDYIKGYNDYSSRVLISITARTKKEAEQFAAILIKVFADLGYDGINVTWTGDTVTVEGSS

SEQ ID NO: 101 - protein, 97 aa, Artificial Sequence
  Description of Artificial Sequence Synthetic polypeptide
VQVTRYYPYSGSADNYYAYTVTTESELQKVLNELKDYIKGYDWASGRVLISITARTKKEAEQFAAILIKVFADLGLLSINVTWTGDTVTVEGYYLEE

SEQ ID NO: 102 - protein, 94 aa, Artificial Sequence
  Description of Artificial Sequence Synthetic polypeptide
VQVTRYYPYSGSADNYYAYTVTTESELQKVLNELKDYIKGYDWASGRVLISITARTKKEAEQFAAILIKVFADLGLLSINVTWTGDTVTVEGYY

SEQ ID NO: 103 - protein, 93 aa, Artificial Sequence
  Description of Artificial Sequence Synthetic polypeptide
VQVTRLRNAYLSDSYYAYTVTTESELQKVLNELKDYIKGYDWASGRVLISITARTKKEAEQFAAILIKVFADLGLLSINVTWTGDTVTVEGYY

SEQ ID NO: 104 - protein, 94 aa, Artificial Sequence
  Description of Artificial Sequence Synthetic polypeptide
VQVTRLNSGGAAYDNYYAYTVTTESELQKVLNELKDYIKSIRIASLRVYISITARTKKEAEQFAAILIKVFADLGLASINVTWTGDTVTVEGYY

SEQ ID NO: 105 - protein, 94 aa, Artificial Sequence
  Description of Artificial Sequence Synthetic polypeptide
VQVTRYNYALNGYDDYYAYTVTTESELQKVLNELKDYIKYRRSYNSGVLISITARTKKEAEQFAAILIKVFADLGYLAINITWTGDTVTVEGYL

SEQ ID NO: 106 - protein, 95 aa, Artificial Sequence
  Description of Artificial Sequence Synthetic polypeptide
VQVTRYNAYARWNDYYYAYTVTTESELQKVLNELKDYIKYLRSYSSDVGISITARTKKEAEQFAAILIKVFADLGYYNINVTWTGDTVTVEGQLV

SEQ ID NO: 107 - protein, 94 aa, Artificial Sequence
  Description of Artificial Sequence Synthetic polypeptide
VQVTRWNASDYGYDSYYAYTVTTESELQKVLNELKDYIKWSYLYNLDVNISITARTKKEAEQFAAILIKVFADLGPYNINVTWTGDTVTVEGAL

SEQ ID NO: 108 - protein, 96 aa, Artificial Sequence
  Description of Artificial Sequence Synthetic polypeptide
VQVTRWNASDYGYDSYYAYTVTTESELQKVLNELKDYIKSAASYSRYVSISITARTKKEAEQFAAILIKVFADLGYNDINVTWTGDTVTVEGQLLL

SEQ ID NO: 109 - protein, 86 aa, Artificial Sequence
  Description of Artificial Sequence Synthetic polypeptide
VQVTRYRAGDYGYDDYYAYTVTTESELQKVLNELKDYIKYAYSYGLAVRISITARTKKEAEQFAAILIKVFADLGYRYINVTWTGD

SEQ ID NO: 110 - protein, 94 aa, Artificial Sequence
  Description of Artificial Sequence Synthetic polypeptide
VQVTRWNSYPSPYDNYYAYTVTTESELQKVLNELKDYIKISASYRYPVAISITARTKKEAEQFAAILIKVFADLGSYIINVTWTGDTVTVEGAP

SEQ ID NO: 111 - protein, 97 aa, Artificial Sequence
  Description of Artificial Sequence Synthetic polypeptide
VQVTRWNSSPNGYDNYYAYTVTTESELQKVLNELKDYIKWGRSYASPVAISITARTKKEAEQFAAILIKVFADLGYDDINVTWTGDTVTVEGQLVLE

SEQ ID NO: 112 - protein, 94 aa, Artificial Sequence
  Description of Artificial Sequence Synthetic polypeptide
VQVTRANRIPYAYDGYYAYTVTTESELQKVLNELKDYIKDYSDPRPAVAISITARTKKEAEQFAAILIKVFADLGYADINVTWTGDTVTVEGLL

SEQ ID NO: 113 - protein, 91 aa, Artificial Sequence
  Description of Artificial Sequence Synthetic polypeptide
VQVNIDDNGKNFDYAYTVTTESELQKVLNELKDYIKSWDLYLPGVNISITARTKKEAEQFAAILIKVFADLGYYDINVTWTGDTVTVGGAA

SEQ ID NO: 114 - protein, 91 aa, Artificial Sequence
  Description of Artificial Sequence Synthetic polypeptide
VQVNIDDNGKNFDYAYTVTTESELQKVLNELKDYIKSWDLYLPGVNISITARTKKEAEQFAAILIKVFADLGYYDINVTWTGDTVTVEGAA

SEQ ID NO: 115 - protein, 93 aa, Artificial Sequence
  Description of Artificial Sequence Synthetic polypeptide
VQVNIDDNGKNFDYAYTVTTESELQKVLNELKDYIKPANLYSGRVLISITARTKKEAEQFAAILIKVFADLGYIDINVTWTGDTVTVEGSALE

SEQ ID NO: 116 - protein, 93 aa, Artificial Sequence
  Description of Artificial Sequence Synthetic polypeptide
VQVTRRPDPARNLDYYAYTVTTESELQKVLNELKDYIKPPIDYRSAVSISITARTKKEAEQFAAILIKVFAELGYLDINVTWTGDTVTVEGSP

SEQ ID NO: 117 - protein, 92 aa, Artificial Sequence
  Description of Artificial Sequence Synthetic polypeptide
VQVTRGPLPDNYDCYYAYTVTTESELQKVLNELKDYIKPCEYSALRVSISITARTKKEAEQFAAILIKVFADLGLLPINVTWTGDTVTVEGL

SEQ ID NO: 118 - protein, 94 aa, Artificial Sequence
  Description of Artificial Sequence Synthetic polypeptide
VQVTRPSAPDRAYDRYYAYTVTTESELQKVLNELKDYIKPPRNYPPYVYISITARTKKEAEQFAAILIKVFADLGYYSINVTWTGDTVTVEGNY

SEQ ID NO: 119 - protein, 95 aa, Artificial Sequence
  Description of Artificial Sequence Synthetic polypeptide
VQVTRSSSPSGSRDYYYAYTVTTESELQKVLNELKDYIKAPLYPYLAVYISITARTKKEAEQFAAILIKVFADLGYDPINVTWTGDTVTVEGALL

SEQ ID NO: 120 - protein, 93 aa, Artificial Sequence
  Description of Artificial Sequence Synthetic polypeptide
VQVTRAPYSNSSNDYYYAYTVTTESELQKVLNELKDYIKYRLYNYLLVYISITARTKKEAEQFAAILIKVFADLGGNLINVTWTGDTVTVEGY

SEQ ID NO: 121 - protein, 94 aa, Artificial Sequence
  Description of Artificial Sequence Synthetic polypeptide
VQVTRRPNNSDSNDYYYAYTVTTESELQKVLNELKDYIKNPDDPLPLVYISITARTKKEAEQFAAILIKVFADLGYPRINVTWTGDTVTVEGSW

SEQ ID NO: 122 - protein, 94 aa, Artificial Sequence
  Description of Artificial Sequence Synthetic polypeptide
VQVTRPYYNSYYDDAYYAYTVTTESELQKVLNELKDYIKALYYPLSPVYISITARTKKEAEQFAAILIKVFADLGYPDINVTWTGDTVTVEGRW

SEQ ID NO: 123 - protein, 95 aa, Artificial Sequence
  Description of Artificial Sequence Synthetic polypeptide
VQVTRPSASDWLLDPYYAYTVTTESELQKVLNELKDYIKLDLAPAPRVYISITARTKKEAEQFAAILIKVFADLGYNDINVTWTGDTVTVEGQLV

SEQ ID NO: 124 - protein, 94 aa, Artificial Sequence
  Description of Artificial Sequence Synthetic polypeptide
VQVTRGLWWSINRDAYYAYTVTTESELQKVLNELKDYIKYAYDYSSPVAISITARTKKEAEQFAAILIKVFADLGLLYINVTWTGDTVTVEGWR

SEQ ID NO: 125 - protein, 95 aa, Artificial Sequence
  Description of Artificial Sequence Synthetic polypeptide
VQVTRAPYDDPPYDYYYAYTVTTESELQKVLNELKDYIKLAADYAYPVSISITARTKKEAEQFAAILIKVFADLGGLSINVTWTGDTVTVEGYLL

SEQ ID NO: 126 - protein, 93 aa, Artificial Sequence
  Description of Artificial Sequence Synthetic polypeptide
VQVTRWNASDYGYDSYYAYTVTTESELQKVLNELKDYIKLYDYYPADVRISITARTKKEAEQFAAILIKVFADLGSRINVTWTGDTVTVEGYY

SEQ ID NO: 127 - protein, 93 aa, Artificial Sequence
  Description of Artificial Sequence Synthetic polypeptide
VQVTRLDRSLSAWDYYYAYTVTTESELQKVLNELKDYIKLYDYYPADVRISITARTKKEAEQFAAILIKVFADLGSRINVTWTGDTVTVEGYY

SEQ ID NO: 128 - protein, 97 aa, Artificial Sequence
  Description of Artificial Sequence Synthetic polypeptide
VQVTRILRYRSGYDYYYAYTVTTESELQKVLNELKDYIKSYLYLSLNVWISITARTKKEAEQFAAILIKVFADLGDDLINVTWTGDTVTVEGLALEE

SEQ ID NO: 129 - protein, 94 aa, Artificial Sequence
  Description of Artificial Sequence Synthetic polypeptide
VQVTRYYPYSGSADNYYAYTVTTESELQKVLNELKDYIKALPPDYWYVYISITARTKKEAEQFAAILIKVFADLGPYAINVTWTGDTVTVEGYR

SEQ ID NO: 130 - protein, 94 aa, Artificial Sequence
  Description of Artificial Sequence Synthetic polypeptide
VQVTRNPPWPASYDSYYAYTVTTESELQKVLNELKDYIKYLPPDYWSVSISITARTKKEAEQFAAILIKVFADLGYYLINVTWTGDTVTVEGSS

SEQ ID NO: 131 - protein, 94 aa, Artificial Sequence
  Description of Artificial Sequence Synthetic polypeptide
VQVTRSAYSNISYDSYYAYTVTTESELQKVLNELKDYIKSPWYRDASVNISITARTKKEAEQFAAILIKVFADLGYYAINVTWTGDTVTVEGWW

SEQ ID NO: 132 - protein, 94 aa, Artificial Sequence
  Description of Artificial Sequence Synthetic polypeptide
VQVTRLASNALNRDSYYAYTVTTESELQKVLNELKDYIKPWPYSALRVNISITARTKKEAEQFAAILIKVFADLGLININVTWTGDTVTVEGYN

SEQ ID NO: 133 - protein, 94 aa, Artificial Sequence
  Description of Artificial Sequence Synthetic polypeptide
VQVTRLASNALNRDSYYAYTVTTESELQKVLNELKDYIKPWPYSALRVNISITARTKKEAEQFAAILIKVFADLGYYGINVTWTGDTVTVEGWS

SEQ ID NO: 134 - protein, 94 aa, Artificial Sequence
  Description of Artificial Sequence Synthetic polypeptide
VQVTRLASNALNRDSYYAYTVTTESELQKVLNELKDYIKLALNDGDRVNISITARTKKEAEQFAAILIKVFADLGLLNINVTWTGDTVTVEGYL

SEQ ID NO: 135 - protein, 93 aa, Artificial Sequence
  Description of Artificial Sequence Synthetic polypeptide
VQVTRLASNALNRDSYYAYTVTTESELQKVLNELKDYIKSLRGAYNVNISITARTKKEAEQFAAILIKVFADLGLASINVTWTGDTVTVEGYA

SEQ ID NO: 136 - protein, 94 aa, Artificial Sequence
  Description of Artificial Sequence Synthetic polypeptide
VQVTRLAYNYLPDDGYYAYTVTTESELQKVLNELKDYIKDYYGSPYAVYISITARTKKEAEQFAAILIKVFADLGIYYINVTWTGDTVTVEGSR

SEQ ID NO: 137 - protein, 96 aa, Artificial Sequence
  Description of Artificial Sequence Synthetic polypeptide
VQVTRWYNNYPPLDRYYAYTVTTESELQKVLNELKDYIKYSDRALWYVSISITARTKKEAEQFAAILIKVFADLGLLPINVTWTGDTVTVEGWLLE

SEQ ID NO: 138 - protein, 94 aa, Artificial Sequence
  Description of Artificial Sequence Synthetic polypeptide
VQVTRYARNADYWDNYYAYTVTTESELQKVLNELKDYIKLPSSWYPYVAISITARTKKEAEQFAAILIKVFADLGPYYINVTWTGDTVTVEGIL

SEQ ID NO: 139 - protein, 94 aa, Artificial Sequence
  Description of Artificial Sequence Synthetic polypeptide
VQVTRNRASSAPDDLYYAYTVTTESELQKVLNELKDYIKSNRASYYSVAISITARTKKEAEQFAAILIKVFADLGAANINVTWTGDTVTVEGAY

SEQ ID NO: 140 - protein, 94 aa, Artificial Sequence
  Description of Artificial Sequence Synthetic polypeptide
VQVTRNPRGLAAYDLYYAYTVTTESELQKVLNELKDYIKPSGSDYYYVIISITARTKKEAEQFAAILIKVFADLGLASINVTWTGDTVTVEGYA

SEQ ID NO: 141 - protein, 96 aa, Artificial Sequence
  Description of Artificial Sequence Synthetic polypeptide
VQVTRGYPLGSAPDGYYAYTVTTESELQKVLNELKDYIKDSISSYGYVAISITARTKKEAEQFAAILIKVFADLGLSSINVTWTGDTVTVEGYALE

SEQ ID NO: 142 - protein, 96 aa, Artificial Sequence
  Description of Artificial Sequence Synthetic polypeptide
VQVTRYAYRSSPYDSYYAYTVTTESELQKVLNELKDYIKPSDSSSYRVNISITARTKKEAEQFAAILIKVFADLGYLSINVTWTGDTVTVEGYALE

SEQ ID NO: 143 - protein, 94 aa, Artificial Sequence
  Description of Artificial Sequence Synthetic polypeptide
VQVTRYPYRGGAYDYYYAYTVTTESELQKVLNELKDYIKISYSYGYIVSISITARTKKEAEQFAAILIKVFADLGNSYINVTWTGDTVTVEGSA

SEQ ID NO: 144 - protein, 94 aa, Artificial Sequence
  Description of Artificial Sequence Synthetic polypeptide
VQVTRYYWGANGYDNYYAYTVTTESELQKVLNELKDYIKSSSSLGYYVYISITARTKKEAEQFAAILIKVFADLGYGSINVTWTGDTVTVEGYS

SEQ ID NO: 145 - protein, 93 aa, Artificial Sequence
  Description of Artificial Sequence Synthetic polypeptide
VQVTRYSRSSGDYDAYYAYTVTTESELQKVLNELKDYIKYLRWSSGGVRISITARTKKEAEQFAAILIKVFADLGRSIINVTWTGDTVTVEGN

SEQ ID NO: 146 - protein, 94 aa, Artificial Sequence
  Description of Artificial Sequence Synthetic polypeptide
VQVTRYSAIYPRYDRYYAYTVTTESELQKVLNELKDYIKALGPGGYGVNISITARTKKEAEQFAAILIKVFADLGLSSINVTWTGDTVTVEGYA

SEQ ID NO: 147 - protein, 96 aa, Artificial Sequence
  Description of Artificial Sequence Synthetic polypeptide
VQVTRYDSIPAGLDRYYAYTVTTESELQKVLNELKDYIKYYSYYRYWVGISITARTKKEAEQFAAILIKVFADLGWSWINVTWTGDTVTVERSSVI

SEQ ID NO: 148 - protein, 94 aa, Artificial Sequence
  Description of Artificial Sequence Synthetic polypeptide
VQVTRYPSYPADPDYYYAYTVTTESELQKVLNELKDYIKYWSYYSSYVNISITARTKKEAEQFAAILIKVFADLGLSSINVTWTGDTVTVEGYY

SEQ ID NO: 149 - protein, 95 aa, Artificial Sequence
  Description of Artificial Sequence Synthetic polypeptide
VQVTRPLSYSDNPDYYYAYTVTTESELQKVLNELKDYIKSSNLDGYRVYISITARTKKEAEQFAAILIKVFADLGLWYINVLGPVNSNHRRSSVI

SEQ ID NO: 150 - protein, 93 aa, Artificial Sequence
  Description of Artificial Sequence Synthetic polypeptide
VQVTRLNAASSLGDRYYAYTVTTESELQKVLNELKDYIKWSRSYNSGVNISITARTKKEAEQFAAILIKVFADLGYYSRHLDRHSNSRRTILI

SEQ ID NO: 151 - protein, 90 aa, Artificial Sequence
  Description of Artificial Sequence Synthetic polypeptide
VQVTRSDPSLSSWDYYYAYTVTTESELQKVLNELKDYIKPAILYRGGVLISITARSKKEAEQFAEFLIKVFGPRIAFYRHLDGVTQGKES

SEQ ID NO: 152 - protein, 58 aa, Artificial Sequence
  Description of Artificial Sequence Synthetic polypeptide
VQVTRLPSILDYDDSYYAYTVTTESELQKVLNELKDYIKGYDWASGRVLISITVRTKK

SEQ ID NO: 153 - protein, 95 aa, Artificial Sequence
  Description of Artificial Sequence Synthetic polypeptide
NVQVTRRSARSLLDDRYYAYTVTTESELQKVLNELKDYIKNWYYSSYNVLISITARTKKEAEQFAAILIKVFADLGYAYINVTWTGDTVTVEGDP

SEQ ID NO: 154 - protein, 95 aa, Artificial Sequence
  Description of Artificial Sequence Synthetic polypeptide
NVQVTRRSARSLLDDRYYAYTVTTESELQKVLNELKDYIKNWYYSSYNVLISITARTKKEAEQFAAILIKVFADLGYAYINVTWTGDTVTVEGAP

SEQ ID NO: 155 - protein, 96 aa, Artificial Sequence
  Description of Artificial Sequence Synthetic polypeptide
NVQVTRRSARSLLDDRYYAYTVTTESELQKVLNELKDYIKNWYYSSYNVLISITARTKKEAEQFAAILIKVFADLGYNDINVTWTGDTVTVEGQLV

SEQ ID NO: 156 - protein, 95 aa, Artificial Sequence
  Description of Artificial Sequence Synthetic polypeptide
NVQVTRRRNSASADDLYYAYTVTTESELQKVLNELKDYIKNWYYSYLNVYISITARTKKEAEQFAAILIKVFADLGWRIINVTWTGDTVTVEGLW

SEQ ID NO: 157 - protein, 95 aa, Artificial Sequence
  Description of Artificial Sequence Synthetic polypeptide
NVQVTRRSPSRSPDDLYYAYTVTTESELQKVLNELKDYIKNWYYSYLNVYISITARTKKEAEQFAAILIKVFADLGWRIINVTWTGDTVTVEGLW

SEQ ID NO: 158 - protein, 95 aa, Artificial Sequence
  Description of Artificial Sequence Synthetic polypeptide
NVQVTRSSARPYYDDRYYAYTVTTESELQKVLNELKDYIKNWYYSYLNVYISITARTKKEAEQFAAILIKVFADLGWRIINVTWTGDTVTVEGLW

SEQ ID NO: 159 - protein, 95 aa, Artificial Sequence
  Description of Artificial Sequence Synthetic polypeptide
NVQVTRRRNSASADDLYYAYTVTTESELQKVLNELKDYIKWSYYAYLNVNISITARTKKEAEQFAAILIKVFADLGWYSINVTWTGDTVTVEGWL

SEQ ID NO: 160 - protein, 95 aa, Artificial Sequence
  Description of Artificial Sequence Synthetic polypeptide
NVQVTRRRNSTSADDLYYAYTVTTESELQKVLNELKDYIKWSYYAYLNVNISITARTKKEAEQFAAILIKVFADLGWYSINVTWTGDTVTVEGYY

SEQ ID NO: 161 - protein, 95 aa, Artificial Sequence
  Description of Artificial Sequence Synthetic polypeptide
NVQVTRRRNSASADDLYYAYTVTTESELQKVLNELKDYIKWSYYAYLNVNISITARTKKEAEQFAAILIKVFADLGWYSINVTWTGDTVTVEGYY

SEQ ID NO: 162 - protein, 96 aa, Artificial Sequence
  Description of Artificial Sequence Synthetic polypeptide
NVQVTRRRNSASADDLYYAYTVTTESELQKVLNELKDYIKWSYYAYLNVNISITARTKKEAEQFAAILIKVFADLGYNDINVTWTGDTVTVEGQLV

SEQ ID NO: 163 - protein, 94 aa, Artificial Sequence
  Description of Artificial Sequence Synthetic polypeptide
NVQVTRRRNSASADDLYYAYTVTTESELQKVLNELKDYIKWSYYAYLNVNISITARTKKEAEQFAAILIKVFADLGYAINVTWTGDTVTVEGIR

SEQ ID NO: 164 - protein, 94 aa, Artificial Sequence
  Description of Artificial Sequence Synthetic polypeptide
NVQVTRSDYRAASDYYYAYTVTTESELQKVLNELKDYIKYSRDYPIGVSISITARTKEEAEQFAAILIKVFADLGNSNINVTWTGDTVTVEGGW

SEQ ID NO: 165 - protein, 95 aa, Artificial Sequence
  Description of Artificial Sequence Synthetic polypeptide
NVQVTRADYARPYYDGYYAYTVTTESELQKVLNELKDYIKYTRSYPAYVIISITARTKKEAEQFAAILIKVFADLGLININVTWTGDTVTVEGAW

SEQ ID NO: 166 - protein, 94 aa, Artificial Sequence
  Description of Artificial Sequence Synthetic polypeptide
NVQVTRYLANLAYYDYYAYTVTTESELQKVLNELKDYIKYHSYWSIYVSISITARTKKEAEQFAAILIKVFADLGLSDINVTWTGDTVTVEGDY

SEQ ID NO: 167 - protein, 95 aa, Artificial Sequence
  Description of Artificial Sequence Synthetic polypeptide
NVQVTRSISYYGSADSYYAYTVTTESELQKVLNELKDYIKYGDSYPLLVYISITARTKKEAEQFAAILIKVFADLGASSINVTWTGDTVTVEGIW

SEQ ID NO: 168 - protein, 95 aa, Artificial Sequence
  Description of Artificial Sequence Synthetic polypeptide
NVQVTRYPWRLDRADYYYAYTVTTESELQKVLNELKDYIKWAYDNRNAVRISITARTKKEAEQFAAILIKVFADLGHSYINVTWTGDTVTVEGYY

SEQ ID NO: 169 - protein, 95 aa, Artificial Sequence
  Description of Artificial Sequence Synthetic polypeptide
NVQVTRAISSDSWADYYYAYTVTTESELQKVLNELKDYIKNALDPRRRVYISITARTKKEAEQFAAILIKVFADLGRSYINVTWTGDTVTVEGRA

SEQ ID NO: 170 - protein, 92 aa, Artificial Sequence
  Description of Artificial Sequence Synthetic polypeptide
NVRVTRDSAYRSPRDSYAYTVTTESELQKVLNELKDYIKKQGAKRVRISITACTKKEAEQFAAILIKVFADLGPYSINVTWTGDTVTVEGSS

SEQ ID NO: 171 - protein, 92 aa, Artificial Sequence
  Description of Artificial Sequence Synthetic polypeptide
NVQVTRDSAYRSPRDSYAYTVTTESELQKVLNELKDYIKKQGAKRVRISITARTKKEAEQFAAILIKVFADLGPYSINVTWTGDTVTVEGSS

SEQ ID NO: 172 - protein, 92 aa, Artificial Sequence
  Description of Artificial Sequence Synthetic polypeptide
NVQVTRDSAYRSPRDSYAYTVTTESELQKVLNELKDYIKKQGAKRVRISITARTKKEAEQFAAILIKVFADLGAYDINVTWTGDTVTVEGRW

SEQ ID NO: 173 - protein, 91 aa, Artificial Sequence
  Description of Artificial Sequence Synthetic polypeptide
NVQVTRDSAYRSPRDSYAYTVTTESELQKVLNELKDYIKKQGAKRVRISITARTKKEAEQFAAILIKVFADLGYAINVTWTGDTVTVEGLA

SEQ ID NO: 174 - protein, 92 aa, Artificial Sequence
  Description of Artificial Sequence Synthetic polypeptide
NVQVTRDSAYRSPRDSYAYTVTTESELQKVLNELKDYIKKQGAKRVRISITARTKKEAEQFAAILIKVFADLGLSAINVTWTGDTVTVEGYA

SEQ ID NO: 175 - protein, 92 aa, Artificial Sequence
  Description of Artificial Sequence Synthetic polypeptide
NVQVTRDSAYRSPRDSYAYTVTTESELQKVLNELKDYIKKQGAKRVRISITARTKKEAEQFAAILIKVFADLGSLWINVTWTGDTVTVEGYR

SEQ ID NO: 176 - protein, 95 aa, Artificial Sequence
  Description of Artificial Sequence Synthetic polypeptide
NVQVTRGSADYPRSDSYYAYTVTTESELQKVLNELKDYIKSNYGSPGRVSISITARTKKEAEQFAAILIKVFADLGLYYINVTWTGDTVTVEGYL

SEQ ID NO: 177 - protein, 94 aa, Artificial Sequence
  Description of Artificial Sequence Synthetic polypeptide
NVQVTRGGSYNRSDNYYAYTVTTESELQKVLNELKDYIKSNYGSPGRVSISITARTKKEAEQFAAILIKVFADLGLYYINVTWTGDTVTVEGYL

SEQ ID NO: 178 - protein, 95 aa, Artificial Sequence
  Description of Artificial Sequence Synthetic polypeptide
NVQVTRSNASNPRIDAYYAYTVTTESELQKVLNKLKDYIKSNYGSPGRVSISITARTKKEAEQFAAILIKVFADLGLYYINVTWTGDTVTVEGYL

SEQ ID NO: 179 - protein, 92 aa, Artificial Sequence
  Description of Artificial Sequence Synthetic polypeptide
NVQVTRGGSYNRSDNYYAYTVTTESELQKVLNELKDYIKKQGAKRVRISITARTKKEAEQFAAILIKVFADLGLYYINVTWTGDTVTVEGYA

SEQ ID NO: 180 - protein, 95 aa, Artificial Sequence
  Description of Artificial Sequence Synthetic polypeptide
NVQVTRSNAYNRRADYYYAYTVTTESELQKVLNELKDYIKSSNYSNGRVIISITARTKKEAEQFAAILIKVFADLGSYAINVTWTGDTVTVEGWW

SEQ ID NO: 181 - protein, 93 aa, Artificial Sequence
  Description of Artificial Sequence Synthetic polypeptide
NVQVTRSNAYNRRADYYYAYTVTTESELQKVLNELKNYIKSSNYSNGRVIISITARTKKKAEQFAAILIKVFADLGSYAINVTWTGDTVTVKG

SEQ ID NO: 182 - protein, 95 aa, Artificial Sequence
  Description of Artificial Sequence Synthetic polypeptide
NVQVTRSNAYNRRADYYYAYTVTTESELQKVLNELKDYIKSSNYSNGRVIISITARTKEEAEQFAAILIKVFADLGSYAINVTWTGDTVTVEGWW

SEQ ID NO: 183 - protein, 95 aa, Artificial Sequence
  Description of Artificial Sequence Synthetic polypeptide
NVQVTRSNAYNRRADYYYAYTVTTESELQKVLNELKDYIKPAAIYADSVPISITARTKKEAEQFAAILIKVFADLGSNYINVTWTGDTVTVEGRS

SEQ ID NO: 184 - protein, 94 aa, Artificial Sequence
  Description of Artificial Sequence Synthetic polypeptide
NVQVTRRSGYNYSSDYYAYTVTTESELQKVLNELKDYIKLLPYPRNIVYISITARTKKEAEQFAAILIKVFADLGAYDINVTWTGDTVTVEGYL

SEQ ID NO: 185 - protein, 94 aa, Artificial Sequence
  Description of Artificial Sequence Synthetic polypeptide
NVQVTRRSGYNYSSDYYAYTVTTESELQKALNELKDYIKLLPYPRNIVYISITARTKKEAEQFAAILIKVFADLGAYDINVTWTGDTVTVEGYL

SEQ ID NO: 186 - protein, 95 aa, Artificial Sequence
  Description of Artificial Sequence Synthetic polypeptide
NVQITRGADYSRAGDYYYAYTVTTESELQKVLNELKDYIKYRDLSYNYVLISITARTKKEAEQFAAILIKVFADLGLYNINVTWTGDTVTVEGLL

SEQ ID NO: 187 - protein, 95 aa, Artificial Sequence
  Description of Artificial Sequence Synthetic polypeptide
NVQVTRYWGYSPPYDAYYAYTVTTESELQKVLNELKDYIKLAYRNALIVRISITARTKKEAEQFAAILIKVFADLGNYSINVTWTGDTVTVEGSR

SEQ ID NO: 188 - protein, 95 aa, Artificial Sequence
  Description of Artificial Sequence Synthetic polypeptide
NVQVTRSDPLYPLYDAYYAYTVTTESELQKVLNELKDYIKIRYPLISAVYISITARTKKEAEQFAAILIKVFADLGYYRINVTWTGDTVTVKGSS

SEQ ID NO: 189 - protein, 94 aa, Artificial Sequence
  Description of Artificial Sequence Synthetic polypeptide
NVQVTRSYSNPADDYYYAYTVTTESELQKVLNELKDYIKSSGYYNLPVRISITARTKKEAEQFAAILIKVFADLGGNSINVTWTGDTVTVEGAA

SEQ ID NO: 190 - protein, 92 aa, Artificial Sequence
  Description of Artificial Sequence Synthetic polypeptide
NVQVNIDDNGKNFDYAYTVTTESELQKVLNELKDYIKLSGWRNPPVYISITARTKKEAEQFAAILIKVFADLGYYNINVTWTGDTVTVEGNS

SEQ ID NO: 191 - protein, 91 aa, Artificial Sequence
  Description of Artificial Sequence Synthetic polypeptide
NVQVNIDDNGKNFDYAYTVTTESELQKVLNELKDYIKYNNYGYYSVAISITARTKKEAEQFAAILIKVFADLGYAINVTWTGDTVTVEGLA

SEQ ID NO: 192 - protein, 93 aa, Artificial Sequence
  Description of Artificial Sequence Synthetic polypeptide
NVQVTRDPRRPGGDYYAYTVTTESELQKVLNELKDYIKPSSILPYYVSISITARTKKEAEQFAAILIKVFADLGPYSINVTWTGDTVTVEGNY

SEQ ID NO: 193 - protein, 91 aa, Artificial Sequence
  Description of Artificial Sequence Synthetic polypeptide
VQVTRDSAYRSPRDSYAYTVTTESELQKVLNELKDYIKKQGAKRVRISITARTKKEAEQFAAILIKVFADLGSYAINVTWTGDTVTVEGWW

SEQ ID NO: 194 - protein, 94 aa, Artificial Sequence
  Description of Artificial Sequence Synthetic polypeptide
VQVTRYAAAYDIPDAYYAYTVTTESELQKVLNELKDYIKLAADNASLVAISITARTKKEAEQFAAILIKVFADLGPYNINVTWTGDTVTVEGLR

SEQ ID NO: 195 - protein, 94 aa, Artificial Sequence
  Description of Artificial Sequence Synthetic polypeptide
VQVTRGNYWDDSYDNYYAYTVTTESELQKVLNELKDYIKAAAALDAYVRISITARTKKEAEQFAAILIKVFADLGPYNINVTWTGDTVTVEGLR

SEQ ID NO: 196 - protein, 95 aa, Artificial Sequence
  Description of Artificial Sequence Synthetic polypeptide
VQVTRSLNWPAPYDLYYAYTVTTESELQKVLNELKDYIKLAADYAYPVSISITARTKKEAEQFAAILIKVFADLGYLYINVTWTGDTVTVEGLYR

SEQ ID NO: 197 - protein, 94 aa, Artificial Sequence
  Description of Artificial Sequence Synthetic polypeptide
VQVTRLGNISSGIDSYYVYTVTTESELQKVLNELKDYIKLDLYPSSSVLISITARTKKEAEQFAAILIKVFADLGAANINVTWTGDTVTVEGLY

SEQ ID NO: 198 - protein, 94 aa, Artificial Sequence
  Description of Artificial Sequence Synthetic polypeptide
VQVTRLGNGNSYADYYYAYTVTTESELQKVLNELKDYIKYPIYGSYRVLISITARTKKEAEQFAAILIKVFADLGAYPINVTWTGDTVTVEGSI

SEQ ID NO: 199 - protein, 94 aa, Artificial Sequence
  Description of Artificial Sequence Synthetic polypeptide
VQVTRALILNIDDDDYYAYTVTTESELQKVLNELKDYIKYYAWDNNAVYISITARTKKEAEQFAAILIKVFADLGRYLINVTWTGDTVTVEGRR

SEQ ID NO: 200 - protein, 96 aa, Artificial Sequence
  Description of Artificial Sequence Synthetic polypeptide
VQVTRSWSDAIAYDDYYAYTVTTESELQKVLNELKDYIKDYALDSAYVAISITARTKKEAEQFAAILIKVFADLGLYGINVTWTGDTVTVEGPEVI

SEQ ID NO: 201 - protein, 94 aa, Artificial Sequence
  Description of Artificial Sequence Synthetic polypeptide
VQVTRRPLNLNSPDRYYAYTVTTESELQKVLNELKDYIKYYLLADAWVYISITARTKKEAEQFAAILIKVFADLGLYDINVTWTGDTVTVEGYS

SEQ ID NO: 202 - protein, 95 aa, Artificial Sequence
  Description of Artificial Sequence Synthetic polypeptide
VQVTRRPLNLNSPDRYYAYTVTTESELQKVLNELKDYIKYYLLADAWVYISITARTKKEAEQFAAILIKVFADLGLYDINVTGGDTVTVEGKEKI

SEQ ID NO: 203 - protein, 94 aa, Artificial Sequence
  Description of Artificial Sequence Synthetic polypeptide
VQVTRLLYDGNSSDYYYAYTVTTESELQKVLNELKDYIKSDRGAAARVSISITARTKKEAEQFAAILIKVFADLGLYNINVTWTGDTVTVEGEK

SEQ ID NO: 204 - protein, 94 aa, Artificial Sequence
  Description of Artificial Sequence Synthetic polypeptide
VQVTRLNAPNYDYDSYYAYTVTTESELQKVLNELKDYIKSSRNIAGLVYISITARTKKEAEQFAAILIKVFADLGLYNINVTWTGDTVTVEGSL

SEQ ID NO: 205 - protein, 93 aa, Artificial Sequence
  Description of Artificial Sequence Synthetic polypeptide
VQVTRRPNNSDSNDYYYAYTVTTESELQKVLNELKDYIKNPDDPLPLVYISITARTKKEAEQFAAILIKVFADLGYPRINVTWTGDTVTVGSS

SEQ ID NO: 206 - protein, 94 aa, Artificial Sequence
  Description of Artificial Sequence Synthetic polypeptide
VQVTRRPGYNRSYDYYYAYTVTTESELQKVLNELKDYIKYANAYPALVYISITARTKKEAEQFAAILIKVFADLGSLNINVTWTGDTVTVEGSW

SEQ ID NO: 207 - protein, 94 aa, Artificial Sequence
  Description of Artificial Sequence Synthetic polypeptide
VQVTRSRYGSNANDYYYAYTVTTESELQKVLNELKDYIKYSPLALLSVYISITARTKKEAEQFAAILIKVFADLGWANINVTWTGDTVTVEGLP

SEQ ID NO: 208 - protein, 94 aa, Artificial Sequence
  Description of Artificial Sequence Synthetic polypeptide
VQVTRLYYYAPPSDYYYAYTVTTESELQKVLNELKDYIKYYYYYIDYVAISITARTKKEAEQFAAILIKVFADLGWPYINVTWTGDTVTVEGNS

SEQ ID NO: 209 - protein, 97 aa, Artificial Sequence
  Description of Artificial Sequence Synthetic polypeptide
VQVTRNYYYDPASDYYYAYTVTTESELQKVLNELKDYIKYPYYPILNVYISITARTKKGAEQFAAILIKVFADLGYYDINVTWTGDTVTVEGNGSNN

SEQ ID NO: 210 - protein, 94 aa, Artificial Sequence
  Description of Artificial Sequence Synthetic polypeptide
VQVTRDPLSDSSYDYYYAYTVTTESELQKVLNELKDYIKYAYPGPYPVIISITARTKKEAEQFAAILIKVFADLGYADINVTWTGDTVTVEGWP

SEQ ID NO: 211 - protein, 93 aa, Artificial Sequence
  Description of Artificial Sequence Synthetic polypeptide
VQVTRSPNDLSYNDLYYAYTVTTESELQKVLNELKDYIKLGYYAPPGVIISITARTKKEAEQFAAILIKVFADLGYIYINVTRGDTVTVEGYK

SEQ ID NO: 212 - protein, 94 aa, Artificial Sequence
  Description of Artificial Sequence Synthetic polypeptide
VQVTRYYPSYSSIDYYYAYTVTTESELQKVLNELKDYIKALPPDYWYVYISITARTKKEAEQFAAILIKVFADLGYYDINVTWTGDTVTVEGYS

SEQ ID NO: 213 - protein, 94 aa, Artificial Sequence
  Description of Artificial Sequence Synthetic polypeptide
VQVTRYSSAGSNNDYYYAYTVTTESELQKVLNELKDYIKALPPDYWYVYISITARTKKEAEQFAAILIKVFADLGYYDINVTWTGDTVTVEGYS

SEQ ID NO: 214 - protein, 94 aa, Artificial Sequence
  Description of Artificial Sequence Synthetic polypeptide
VQVTRSYNDDNYGDYYYAYTVTTESELQKVLNELKDYIKALPPDYWYVYISITARTKKEAEQFAAILIKVFADLGYPRINVTWTGDTVTVEGYL

SEQ ID NO: 215 - protein, 93 aa, Artificial Sequence
  Description of Artificial Sequence Synthetic polypeptide
VQVTRWNSSANGYDYYYAYTVTTESELQKVLNELKDYIKIAASYSRYVSISITARTKKEAEQFAAILIKVFADLGYYDINVTDRHSNSRRRRE

SEQ ID NO: 216 - protein, 91 aa, Artificial Sequence
  Description of Artificial Sequence Synthetic polypeptide
VQVNIDDNGKNFDYAYTVTTESELQKVLNELKDYIKPANLYSDPVSISITARTKKEAEQFAAILIKVFADLGYYGINVTWTGDTVTVEGYS

SEQ ID NO: 217 - protein, 96 aa, Artificial Sequence
  Description of Artificial Sequence Synthetic polypeptide
VQVTRYRSGSYGYDSYYAYTVTTESELQKVLNELKDYIKYSYSYSYDVYISITARTKKEAEQFAAILIKVFADLGSYDINVTWTGDTVTVEGTLNN

SEQ ID NO: 218 - protein, 94 aa, Artificial Sequence
  Description of Artificial Sequence Synthetic polypeptide
VQVTRLYPLSSPSDYYYAYTVTTESELQKVLNELKDYIKLGSSYSLRVSISITARTKKEAEQFAAILIKVFADLGLASINVTWTGDTVTVEGYW

SEQ ID NO: 219 - protein, 94 aa, Artificial Sequence
  Description of Artificial Sequence Synthetic polypeptide
VQVTRLYPLSSPSDYYYAYTVTTESELQKVLNELKDYIKPANLYSGRVLISITARTKKEAEQFAAILIKVFADLGLSSINVTWTGDTVTVEGYA

SEQ ID NO: 220 - protein, 94 aa, Artificial Sequence
  Description of Artificial Sequence Synthetic polypeptide
VQVTRLAASSRSSDYYYAYTVTTESELQKVLNELKDYIKPANLYSGRVLISITARTKKEAEQFAAILIKVFADLGLSSINVTWTGDTVTVEGYA

SEQ ID NO: 221 - protein, 94 aa, Artificial Sequence
  Description of Artificial Sequence Synthetic polypeptide
VQVTRSYPGASSSDAYYAYTVTTESELQKVLNELKDYIKPANLYSGRVLISITARTKKEAEQFAAILIKVFADLGLSSINVTWTGDTVTVEGYY

SEQ ID NO: 222 - protein, 94 aa, Artificial Sequence
  Description of Artificial Sequence Synthetic polypeptide
VQVTRSYPGASSSDAYYAYTVTTESELQKVLNELKDYIKPANLYSGRVLISITARTKKEAEQFAAILIKVFADLGLSSINVTWTGDTVTVEGYA

SEQ ID NO: 223 - protein, 94 aa, Artificial Sequence
  Description of Artificial Sequence Synthetic polypeptide
VQVTRLLPYADSPDYYYAYTVTTESELQKVLNELKDYIKGYNDYSSRVLISITARTKKEAEQFAAILIKVFADLGWASINVTWTGDTVTVEGYS

SEQ ID NO: 224 - protein, 94 aa, Artificial Sequence
  Description of Artificial Sequence Synthetic polypeptide
VQVTRLYYNSNYNDAYYAYTVTTESELQKVLNELKDYIKGYNDYSSRVLISITARTKKEAEQFAAILIKVFADLGWASINVTWTGDTVTVEGYY

SEQ ID NO: 225 - protein, 94 aa, Artificial Sequence
  Description of Artificial Sequence Synthetic polypeptide
VQVTRLAPNAARPDNYYAYTVTTESELQKVLNELKDYIKYPSLYSYRVNISITARTKKEAEQFAAILIKVFADLGYASINVTWTGDTVTVEGYS

SEQ ID NO: 226 - protein, 93 aa, Artificial Sequence
  Description of Artificial Sequence Synthetic polypeptide
VQVTRLAPNAARPDTYYAYTVTTESELQKVLNKLKNYIKYPTLYSYRVNFSFTARTKKKPNQFAAFLIKVFPNLGYTSINVTTGDTVTVKGYS

SEQ ID NO: 227 - protein, 94 aa, Artificial Sequence
  Description of Artificial Sequence Synthetic polypeptide
VQVTRLSPRYLLSDAYYAYTVTTESELQKVLNELKDYIKPIDNLPYRVIISITARTKKEAEQFAAILIKVFADLGYGSINVTWTGDTVTVEGYS

SEQ ID NO: 228 - protein, 94 aa, Artificial Sequence
  Description of Artificial Sequence Synthetic polypeptide
VQVTRLASNALNRDSYYAYTVTTESELQKVLNELKDYIKPWPYSALRVNISITARTKKEAEQFAAILIKVFADLGYYDINVTWTGDTVTVEGWA

SEQ ID NO: 229 - protein, 95 aa, Artificial Sequence
  Description of Artificial Sequence Synthetic polypeptide
VQVTRYPSYPAYADYYYAYTVTTESELQKVLNELKDYIKYSANPSSRVAISITARTKKEAEQFAAILIKVFADLGLASINVTWTGDTVTVEGQLV

SEQ ID NO: 230 - protein, 96 aa, Artificial Sequence
  Description of Artificial Sequence Synthetic polypeptide
VQVTRYPSYPAYSDYYYAYTVTTESELQKVLNELKDYIKYSDNSSSHVAISITARTKKEAEQFAAILIKVFADLGLAYINVTWTGDTVTVEGQLIL

SEQ ID NO: 231 - protein, 94 aa, Artificial Sequence
  Description of Artificial Sequence Synthetic polypeptide
VQVTRYRSYNPWSDYYYAYTVTTESELQKVLNELKDYIKLWSWGSARVNISITARTKKEAEQFAAILIKVFADLGLISINVTWTGDTVTVEGYA

SEQ ID NO: 232 - protein, 92 aa, Artificial Sequence
  Description of Artificial Sequence Synthetic polypeptide
VQVTRDRSYTLWYDYYYAYTVTTESELQKVLNELKDYIKWSWDSAPVNISITARTKKEAEQFAAILIKVFADLGRISINVTWTGDTVTVEGA

SEQ ID NO: 233 - protein, 94 aa, Artificial Sequence
  Description of Artificial Sequence Synthetic polypeptide
VQVTRYNGPDDGSDYYYAYTVTTESELQKVLNELKDYIKSYYANAYLVYISITARTKKEAEQFAAILIKVFADLGWDSTNVTWTGDTVTVEGRP

SEQ ID NO: 234 - protein, 94 aa, Artificial Sequence
  Description of Artificial Sequence Synthetic polypeptide
VQVTRYPNYSDPSDSYYAYTVTTESELQKVLNELKDYIKDYYGSPYAVYISITARTKKEAEQFAAILIKVFADLGYAYINVTWTGDTVTVEGRW

SEQ ID NO: 235 - protein, 93 aa, Artificial Sequence
  Description of Artificial Sequence Synthetic polypeptide
VQVTRAPAYNAGWDPYYAYTVTTESELQKVLNELKDYIKSGYYYSNSVISITARTKKEAEQFAAILIKVFADLGPWNINVTWTGDTVTVEGGA

SEQ ID NO: 236 - protein, 94 aa, Artificial Sequence
  Description of Artificial Sequence Synthetic polypeptide
VQVTRPYGYSYGSDDYYAYTVTTESELQKVLNELKDYIKPGYAYPSRVNISITARTKKEAEQFAAILIKVFADLGLYSINVTWTGDTVTVEGRY

SEQ ID NO: 237 - protein, 93 aa, Artificial Sequence
  Description of Artificial Sequence Synthetic polypeptide
VQVTRASSYSSYDSYYAYTVTTESELQKVLNELKDYIKIIYSYGGYVYISITARTKKEAEQFAAILIKVFADLGLASINVTWTGDTVTVEGYC

SEQ ID NO: 238 - protein, 92 aa, Artificial Sequence
  Description of Artificial Sequence Synthetic polypeptide
VQVTRWLLNNNYDNYYAYTVTTESELQKVLNELKDYIKSSYLYPSRVNISITARTKKEAEQFAAILIKVFADLGSRINVTWTGDTVTVEGYY

SEQ ID NO: 239 - protein, 91 aa, Artificial Sequence
  Description of Artificial Sequence Synthetic polypeptide
VQVTRDSAYRSGHDPYSYATTKYQLKIVKNDKKDYIKYNNLRYYSVAFFITRRTKKEDELVAAILIKVFDDLRYSINVTWTGDTVTVEGHW

SEQ ID NO: 240 - protein, 91 aa, Artificial Sequence
  Description of Artificial Sequence Synthetic polypeptide
VQVTRDSAYRSPRDSYAYTVTTESELQKVLNELKDYIKKQGAKRVRISITARTKKEAEQFAAILIKVFADLGLGGINVTWTGDTVTVEGLA

SEQ ID NO: 241 - protein, 93 aa, Artificial Sequence
  Description of Artificial Sequence Synthetic polypeptide
VQVTRRSGYNYSSDYYAYTVTTESELRKVLNELKDYIKLLPYPRNIVYISITARTKKEAEQFAAILIKVFADLGAYDINVTWTGDTVTVEGYL

SEQ ID NO: 242 - protein, 93 aa, Artificial Sequence
  Description of Artificial Sequence Synthetic polypeptide
VQVTRRSGYNYSSDYYAYTVTTESELQKVLNELKDYIKLLPYPRNIVYISITARTKKEAEQFAAILIKVFADLGAYDINVTWTGDTVTVEGLW

SEQ ID NO: 243 - protein, 93 aa, Artificial Sequence
  Description of Artificial Sequence Synthetic polypeptide
VQVTRRSGYNYSSDYYAYTVTTESELQKVLNELKDYIKLLPYPRNIVYISITARTKKEAEQFAAILIKVFADLGWYSINVTWTGDTVTVEGWL

SEQ ID NO: 244 - protein, 93 aa, Artificial Sequence
  Description of Artificial Sequence Synthetic polypeptide
VQVTRYRSSYWYNDGYAYTVTTESELQKVLNELKDYIKYYNSNINSVLISITARTKKEAEQFAAILIKVFADLGYYSINVTWTGDTVTVEGNL

SEQ ID NO: 245 - protein, 94 aa, Artificial Sequence
  Description of Artificial Sequence Synthetic polypeptide
VQVTRGAISPWDPDAYYAYTVTTESELQKVLNELKDYIKYSSAYPIDVPISITARTKKEAEQFAAILIKVFADLGALDINVTWTGDTVTVEGRW

SEQ ID NO: 246 - protein, 94 aa, Artificial Sequence
  Description of Artificial Sequence Synthetic polypeptide
VQVTRGAISPWDPDSYYAYTVTTESELQKVLNELKDYIKYSSAYPIDVPISITARTKKEAEQFAAILIKVFADLGPLDINVTWTGDTVTVEGRW

SEQ ID NO: 247 - protein, 94 aa, Artificial Sequence
  Description of Artificial Sequence Synthetic polypeptide
VQVTRDPRRSPYSDYYYAYTVTTESELQKVLNELKDYIKYSSGYPISVPISITARTKKEAEQFAAILIKVFADLGPLIINVTWTGDTVTVEGRW

SEQ ID NO: 248 - protein, 94 aa, Artificial Sequence
  Description of Artificial Sequence Synthetic polypeptide
VQVTRSSRNYSNADYYYAYTVTTESELQKVLNELKDYIKYSSAYPIDVPISITARTKKEAEQFAAILIKVFADLGLININVTWTGDTVTVEGAW

SEQ ID NO: 249 - protein, 94 aa, Artificial Sequence
  Description of Artificial Sequence Synthetic polypeptide
VQVTRSSPRSASYDNYYAYTVTTESELQKVLNELKDYIKYSSSYPLRVDISITARTKKEAEQFAAILIKVFADLGAPSINVTWTGDTVTVEGWW

SEQ ID NO: 250 - protein, 94 aa, Artificial Sequence
  Description of Artificial Sequence Synthetic polypeptide
VQVTRSDAPRLAYDSYYAYTVTTESELQKVLNELKDYIKYTRSYPAYVIISITARTKKEAEQFAAILIKVFADLGLININVTWTGDTVTVEGAW

SEQ ID NO: 251 - protein, 94 aa, Artificial Sequence
  Description of Artificial Sequence Synthetic polypeptide
VQVTRLSSSAYPDDYYYAYTVTTESELQKVLNELKDYIKYSNSYPAAVLISITARTKKEAEQFAAILIKVFADLGGSYINVTWTGDTVTIEGRW

SEQ ID NO: 252 - protein, 94 aa, Artificial Sequence
  Description of Artificial Sequence Synthetic polypeptide
VQVTRNNSYRYLNDSYYAYTVTTESELQKVLNELKDYIKYSYGYPLNVLISITARTKKEAEQLAAILIKVFADLGYGAINVTWTGDTVTVEGNS

SEQ ID NO: 253 - protein, 94 aa, Artificial Sequence
  Description of Artificial Sequence Synthetic polypeptide
VQVTRSYNDYPRADSYYAYTVTTESELQKVLNELKDYIKSNYGSPGRVSISITARTKKEAEQFAAILIIVFADLGLYYINVTWTGDTVTVEGYL

SEQ ID NO: 254 - protein, 94 aa, Artificial Sequence
  Description of Artificial Sequence Synthetic polypeptide
VQITRSYNDYPRADSYYAYTVTTESELRKVLNELKDYIKSNYGSPGRVSISITARTKKEAEQFAAILIKVFADLGWYSINVTWTGDTVTVEGYY

SEQ ID NO: 255 - protein, 94 aa, Artificial Sequence
  Description of Artificial Sequence Synthetic polypeptide
VQVTRSYNDYPRADSYYAYTVTTESELQKVLNELKDYIKSNYGSPGRVSISITARTKKEAEQFAAILIKVFADLGYPRINVTWTGDTVTVEGIA

SEQ ID NO: 256 - protein, 94 aa, Artificial Sequence
  Description of Artificial Sequence Synthetic polypeptide
VQVTRGNYANARIDSYYAYTVTTESELQKVLNELKDYIKLLAYSSARVIISITARTKKEAEQFAAILIKVFADLGAYDINVTWTGDTVTVEGLW

SEQ ID NO: 257 - protein, 94 aa, Artificial Sequence
  Description of Artificial Sequence Synthetic polypeptide
VQVTRSYNDYPPADSYYAYTVTTESELQKVLNELKDYIKLLAYSSARVIISITARTKKEAEQFAAILIKVFADLGSYDINVTWTGDTVTVEGLW

SEQ ID NO: 258 - protein, 94 aa, Artificial Sequence
  Description of Artificial Sequence Synthetic polypeptide
VQVTRSNASNPRIDAYYAYTVTTESELQKVLNELKDYIKSSNYSNGRVIISITARTKKEAEQFAAILIKVFADLGSYAINVTWTGDTVTVEGWW

SEQ ID NO: 259 - protein, 94 aa, Artificial Sequence
  Description of Artificial Sequence Synthetic polypeptide
VQVTRSNASNPRIDAYYAYTVTTESELQKVLNELKDYIKSSNYSNGRVIISITARTKKEAEQFAAILIRVFADLGSYAINVTWTGDTVTVEGWW

SEQ ID NO: 260 - protein, 94 aa, Artificial Sequence
  Description of Artificial Sequence Synthetic polypeptide
VQVTRSNAYNRRADYYYAYTVTTESELQKVLNELEDYIKSSNYSNGRVIISITARTKKEAEQFAAILIKVFADLGSYAINVTWTGDTVTVEGWW

SEQ ID NO: 261 - protein, 94 aa, Artificial Sequence
  Description of Artificial Sequence Synthetic polypeptide
VQVTRRRNSASADDLYYAYTVTTESELQEVLNELKDYIKWSYYAYLNVNISITARTKKEAEQFAAILIKVFADLGWYSINVTWTGGTVTVEGWL

SEQ ID NO: 262 - protein, 94 aa, Artificial Sequence
  Description of Artificial Sequence Synthetic polypeptide
VQVTRRPNSASADDLYYAYTVTTESELQKVLNELKDYIKWSYYAYLNVNISITARTKKEAEQFAAILIKVFADLGWYSINVTWTGDTVTVEGDP

SEQ ID NO: 263 - protein, 94 aa, Artificial Sequence
  Description of Artificial Sequence Synthetic polypeptide
VQVTRRPSSASADDLYYAYTVTTESELQKVLNELKDYIKWSYYPYLNVNISITARTKKEAEQFAAILIKVFADLGWYSINVTWTGDTVTVEGYY

SEQ ID NO: 264 - protein, 94 aa, Artificial Sequence
  Description of Artificial Sequence Synthetic polypeptide
VQVTRRHNSASADDLYYAYTVTTESELQKVLNELKDYIKWSYYAYLNVNISITARTKKEAEQFAAILIKVFADLGLYNINVTWTGDTVTVEGLL

SEQ ID NO: 265 - protein, 92 aa, Artificial Sequence
  Description of Artificial Sequence Synthetic polypeptide
VHVTRLHNSASAHDLHYAYTVTTEAKLQKVLNELKDDIKSYYAYPLVAFSITARTIQTEQFAAILIKVFPDLGWYSINVTWTGDTVTVQGLY

SEQ ID NO: 266 - protein, 94 aa, Artificial Sequence
  Description of Artificial Sequence Synthetic polypeptide
VQVTRGLLYAGYSDSYYAYTVTTESELQKVLNELKDYIKSYYYDIASVSISITARTKKEAEQFAAILIKVFADLGGIPINVTWTGDTVTVEGLD

SEQ ID NO: 267 - protein, 94 aa, Artificial Sequence
  Description of Artificial Sequence Synthetic polypeptide
VQVTRRSARSLLDDRYYAYTVTTESELQKVLNELKDYIKNWYYSSYNVLISITARTKKEAERFAAILIKVFADLGYAYINVTWTGDTVTVEGAN

SEQ ID NO: 268 - protein, 93 aa, Artificial Sequence
  Description of Artificial Sequence Synthetic polypeptide
VQVTRRSARSLLDDRYYAYTVTTESELQKVLNELKDYIKNWYYSSYNVLISITARTKREAEQFAAILIKVFADLGYAINVTWTGDTVTVEGRW

SEQ ID NO: 269 - protein, 94 aa, Artificial Sequence
  Description of Artificial Sequence Synthetic polypeptide
VQVTRRSARSLLDDRYYAYTVTTESELQKVLNELKDYIKNWYYSSYNVLISITARTKKEAEQFAAILIKVFADLGLYNINVTWTGDTVTVEGLL

SEQ ID NO: 270 - protein, 94 aa, Artificial Sequence
  Description of Artificial Sequence Synthetic polypeptide
VQVTRPSARSLLDDRYYAYTVTTESELQKVLNELKDYIKYWYDSSYGVLISITARTKKEAEQFAAILIKVFADLGLYYINVTWTGDTVTVEGLR

SEQ ID NO: 271 - protein, 91 aa, Artificial Sequence
  Description of Artificial Sequence Synthetic polypeptide
VQVTRRSARSLLDDRYYAYTVTTESELQKVLNELKDYIKKQGAKRVRISITARTKKEAEQFAAILIKVFADLGLYYINVTWTGDTVTVEGY

SEQ ID NO: 272 - protein, 93 aa, Artificial Sequence
  Description of Artificial Sequence Synthetic polypeptide
VQVTRRSARSLLDDRYYAYTVTAESELQKVLNELKDYIKYNNYGYYSVAISITARTKKEAEQFAAILIKVFADLGYAINVTWTGDTVTVEGLA

SEQ ID NO: 273 - protein, 94 aa, Artificial Sequence
  Description of Artificial Sequence Synthetic polypeptide
VQVTRRSARSLLDDRYYAYTVTTESELQKVLNELKDYIKWSYYAYLNVNISITARTKKEAEQFAAILIKVFADLGYRLINVTWTGDTVTVEGYP

SEQ ID NO: 274 - protein, 94 aa, Artificial Sequence
  Description of Artificial Sequence Synthetic polypeptide
VQITRGADYSRAGDYYYAYTVTTESELQKVLNELKDYIKYRDLSYNYVLISITARTKKEAEQFAAILIKVFADLGLYNINVTWTGDTVTVEGLL

SEQ ID NO: 275 - protein, 94 aa, Artificial Sequence
  Description of Artificial Sequence Synthetic polypeptide
VQVTRRSARSLLDDRYYAYTVTTESELQKVLNELKDYIKYRDLSYNYVLISITARTKKEAEQFAAILIKVFADLGLYNINVTWTGDTVTVEGLL

SEQ ID NO: 276 - protein, 94 aa, Artificial Sequence
  Description of Artificial Sequence Synthetic polypeptide
VQVTRYNPYSYSSDYYYAYTVTTESELQKVLNELKDYIKLDDRSDNYVSISITARTKKEAEQFAAILIKVFADLGRLYINVTWTGDTVTVEGPR

SEQ ID NO: 277 - protein, 93 aa, Artificial Sequence
  Description of Artificial Sequence Synthetic polypeptide
VQVTRSAPYSDLSDAYYAYTVTTESELQKVLNELKDYIKLSGYYSPVWISITARTKKEAEQFAAILIKVFADLGLYNINVTWTGDTVTVEGYP

SEQ ID NO: 278 - protein, 92 aa, Artificial Sequence
  Description of Artificial Sequence Synthetic polypeptide
VQVTRDPRRPGGDYYAYTVTTESELQKVLNELKDYIKPSSILPYYVSISITARTKKEAEQFAAILIKVFADLGPYSINVTWTGDTVTVEGNY

SEQ ID NO: 279 - protein, 92 aa, Artificial Sequence
  Description of Artificial Sequence Synthetic polypeptide
VQVTRDPRRPGGDYYAYTVTTESELQKVLNELKDYIKPAPLDPSLVLISITARTKKEAEQFAAILIKVFADLGAANINVTWTGDTVTVEGLW

SEQ ID NO: 280 - protein, 92 aa, Artificial Sequence
  Description of Artificial Sequence Synthetic polypeptide
VQVTRDPRRPGGDYYAYTVTTESELQKVLNELKDYIKWAYDNRNAVRISITARTKKEAEQFAAILIKVFADLGHSYINVTWTGDTVTVEGYY

SEQ ID NO: 281 - protein, 94 aa, Artificial Sequence
  Description of Artificial Sequence Synthetic polypeptide
VQVTRDPLNYSSADYYYAYTVTTESELQKVLNELKDYIKYTRSYPAYVIISITARTKKEAEQFAAILIKVFADLGPYSINVTWTGDTVTVEGNY

SEQ ID NO: 282 - protein, 94 aa, Artificial Sequence
  Description of Artificial Sequence Synthetic polypeptide
VQVTRYPWRLDRADYYYAYTVTTESELQKVLNELKDYIKWAYDNRNAVRISITARTKKEAEQFAAILIKVFADLGHSYINVTWTGDTVTVEGYY

SEQ ID NO: 283 - protein, 94 aa, Artificial Sequence
  Description of Artificial Sequence Synthetic polypeptide
VQVTRSGYPWRPADDYYAYTVTTESELQKVLNELKDYIKNAYIAGNAVLISITARTKKEAEQFAAILIKVFADLGDSAINVTWTGDTVTVEGCY

SEQ ID NO: 284 - protein, 93 aa, Artificial Sequence
  Description of Artificial Sequence Synthetic polypeptide
VQVTRAPYPAWIYDSYYAYTVTTESELQKVLNELKDYIKKQGAKRVRISITARTKKEAEQFAAILIKVFADLGAALINVTWTGDTVTVEGLYK

SEQ ID NO: 285 - protein, 94 aa, Artificial Sequence
  Description of Artificial Sequence Synthetic polypeptide
VQVTRSALNAAPLDIYYAYTVTTESELQKVLNELKDYIKPYYPYSWAVYISITARTKKEAEQFAAILIKVFADLGAAAINVTWTGDTVTVEGRL

SEQ ID NO: 286 - protein, 93 aa, Artificial Sequence
  Description of Artificial Sequence Synthetic polypeptide
VQVTRNSDNRSSYDYYYAYTVTTESELQKVLNELKDYIKYRPYLYLGVYISITARTKKKAEQFAAILIKVFADLGIALINVTWTGDTVTLSAL

SEQ ID NO: 287 - protein, 94 aa, Artificial Sequence
  Description of Artificial Sequence Synthetic polypeptide
VQVTRNSDNRSSYDYYYAYTVTTESELQKVLNELKDYIKYRPYLYLGVYISITARTKKEAEQFAAILIKVFADLGIALINVTWTGDTVTVEGSA

SEQ ID NO: 288 - protein, 91 aa, Artificial Sequence
  Description of Artificial Sequence Synthetic polypeptide
VQVNIDDNGKNFDYAYTVTTESELQKVLNELKDYIKLSGWRNPPVYISITARTKKEAEQFAAILIKVFADLGYYNINVTWTGDTVTVEGNS

SEQ ID NO: 289 - protein, 90 aa, Artificial Sequence
  Description of Artificial Sequence Synthetic polypeptide
VQVNIDDNGKNFDYAYTVTTESELQKVLNELKDYIKYNNYGYYSVAISITARTKKEAEQFAAILIKVFADLGYAINVTWTGDTVTVEGLA

SEQ ID NO: 290 - protein, 92 aa, Artificial Sequence
  Description of Artificial Sequence Synthetic polypeptide
VQVTRDSAYRSPRDSYAYTVTTESELQKVLNELKDYIKYNNYGYYSVAISITARTKKEAEQFAAILIKVFADLGYAINVTWTGDTVTVEGLA

SEQ ID NO: 291 - protein, 89 aa, Artificial Sequence
  Description of Artificial Sequence Synthetic polypeptide
VQVTRDSAYRSPRDSYAYTVTTESELQKVLNELKDYIKKQGAKRVRISITARTKKKLSSLRQFLKYSLTDCTTLTSLGPVDTVTVEGYL

SEQ ID NO: 292 - protein, 97 aa, Artificial Sequence
  Description of Artificial Sequence Synthetic polypeptide
MADINVQVTRDPRRPGGDYYAYTVTTESELQKVLNELKDYIKPSSILPYYVSISITARTKKEAEQFAAILIKVFADLGPYSITVTWTGDTVTVEGNY

SEQ ID NO: 293 - protein, 97 aa, Artificial Sequence
  Description of Artificial Sequence Synthetic polypeptide
MADINVQVTRDPRRPGGDYYAYRVTTEDELQKVLNELKDYIKPSSILPYYVSISIDAETKKEAERFAAILIDVFDDLGPYSITVTFTGDTVRVEGNY

SEQ ID NO: 294 - protein, 97 aa, Artificial Sequence
  Description of Artificial Sequence Synthetic polypeptide
MADINVQVTRDPRRPGGDYYAYRVTTEDELQRVLNELRDYIDPSSILPYYVSISIDAETRREAERFAAILIDVFDDLGPYSITVTFTGDTVRVEGNY

SEQ ID NO: 295 - protein, 99 aa, Artificial Sequence
  Description of Artificial Sequence Synthetic polypeptide
MADINVQVTRRYPPPLRYDYYYAYTVTTESELQKVLNELKDYIKRDYILPSNVLISITARTKKEAEQFAAILIKVFADLGDRDITVTFTGDTVTVEGYL

SEQ ID NO: 296 - protein, 99 aa, Artificial Sequence
  Description of Artificial Sequence Synthetic polypeptide
MADINVQVTRRYPPPLRYDYYYAYRVTTEDELQKVLNELKDYIKRDYILPSNVLISIDAETKKEAERFAAILIDVFDDLGDRDITVTFTGDTVRVEGYL

SEQ ID NO: 297 - protein, 99 aa, Artificial Sequence
  Description of Artificial Sequence Synthetic polypeptide
MADINVQVTRRYPPPLRYDYYYAYRVTTEDELQRVLNELRDYIDRDYILPSNVLISIDAETRREAERFAAILIDVFDDLGDRDITVTFTGDTVRVEGYL

SEQ ID NO: 298 - protein, 95 aa, Artificial Sequence
  Description of Artificial Sequence Synthetic polypeptide
MADIQVQVNIDDNGKNFDYTYTVTTESELQKVLNELKDYIKKQGAKRVRISITARTKKEAEKFAAILIKVFAELGYNDITVTFDGDTVTVEGQLE

SEQ ID NO: 299 - protein, 94 aa, Artificial Sequence
  Description of Artificial Sequence Synthetic polypeptide
MADINVQVNIDDNGKNFDYAYTVTTESELQKVLNELKDYIKKQGAKRVRISITARTKKEAEQFAAILIKVFADLGYNDITVTFTGDTVTVEGQL

SEQ ID NO: 300 - protein, 94 aa, Artificial Sequence
  Description of Artificial Sequence Synthetic polypeptide
MADIQVQVNIDDNGKNFDYAYTVTTESELQKVLNELKDYIKKQGAKRVRISITARTKKEAEQFAAILIKVFADLGYNDITVTFTGDTVTVEGQL

SEQ ID NO: 301 - protein, 95 aa, Artificial Sequence
  Description of Artificial Sequence Synthetic polypeptide
MADIQVQVNIDDNGKNFDYAYTVTTESELQKVLNELKDYIKKQGAKRVRISITARTKKEAEQFAAILIKVFADLGYNDITVTFTGDTVTVEGQLE

SEQ ID NO: 302 - protein, 94 aa, Artificial Sequence
  Description of Artificial Sequence Synthetic polypeptide
MADINVQVNIDDNGKNFDYAYRVTTEDELQKVLNELKDYIKKQGAKRVRISIDAETKKEAERFAAILIDVFDDLGYNDITVTFTGDTVRVEGQL

SEQ ID NO: 303 - protein, 94 aa, Artificial Sequence
  Description of Artificial Sequence Synthetic polypeptide
MADINVQVNIDDNGRNFDYAYRVTTEDELQRVLNELRDYIDRQGARRVRISIDAETRREAERFAAILIDVFDDLGYNDITVTFTGDTVRVEGQL

SEQ ID NO: 304 - protein, 94 aa, Artificial Sequence
  Description of Artificial Sequence Synthetic polypeptide
MADINVQVNIDDNGKNFDYAYTVDTESELQKVLNELKDQIKKQGAKRVRISIDARTKKEAEQFAAILQKVFADLGYNDIDVTFTGDTVTVEGQL

SEQ ID NO: 305 - protein, 94 aa, Artificial Sequence
  Description of Artificial Sequence Synthetic polypeptide
MADINVQVNIDDNGRNFDYAYTVDTESELQRVLNELADQIQRQGANRVRISIDARTQREAEQFAAILQQVFADLGYNDIDVTFTGDTVTVEGQL

SEQ ID NO: 306 - protein, 94 aa, Artificial Sequence
  Description of Artificial Sequence Synthetic polypeptide
MADINVQVNIDDNGRNFDYAYTVDTESELQRVLDELADQIQRQGADRVRISVDARTQDEAEQFAAILQQVFADLGYNDIDVTFTGDTVTVEGQL

SEQ ID NO: 307 - protein, 97 aa, Artificial Sequence
  Description of Artificial Sequence Synthetic polypeptide
MADINVQVTRDPRRPGGDYYAYTVTTESELQKVLNELKDYIKPSSILPYYVSISITARTKKEAEQFAAILIKVFADLGPYSITVTFTGDTVTVEGNY

SEQ ID NO: 308 - protein, 94 aa, Artificial Sequence
  Description of Artificial Sequence Synthetic polypeptide
MAKVDITIKIQRDGQEIEIDIRVSTGKELERALQELEKALARAGARNVQITISAENDEQAKELLELIARLLQKLGYKDINVRVQGTEVKIEVRV

SEQ ID NO: 309 - protein, 94 aa, Artificial Sequence; identical to SEQ ID NO: 299
  Description of Artificial Sequence Synthetic consensus sequence

SEQ ID NO: 310 - protein, 6 aa, Artificial Sequence
  Description of Artificial Sequence Synthetic 6xHis tag
HHHHHH

SEQ ID NO: 311 - protein, 5 aa, Artificial Sequence
  Description of Artificial Sequence Synthetic peptide
GGGGS

SEQ ID NO: 312 - protein, 94 aa, Artificial Sequence
  Description of Artificial Sequence Synthetic polypeptide
MADINVQVNIDDNGKNFDYAYTVTTESELQKVLNELKDYIKKQGAKRVRISITARTKKEAEQFAAILIKVFADLGYNDIDVTFTGDTVTVEGQL

SEQ ID NO: 313 - protein, 99 aa, Artificial Sequence
  Description of Artificial Sequence Synthetic polypeptide
MADINVQVTRNNNSDSWSDIYYAYTVTTESELQKVLNELKDYIKSDGDDYSNVRISITARTKKEAEQFAAILIKVFADLGWYPIDVTFTGDTVTVEGSL

SEQ ID NO: 314 - protein, 99 aa, Artificial Sequence
  Description of Artificial Sequence Synthetic polypeptide
MADINVQVTRGLSSAYLYDYYYAYTVTTESELQKVLNELKDYIKSLLSSYALVYISITARTKKEAEQFAAILIKVFADLGPGAIDVTFTGDTVTVEGSL

SEQ ID NO: 315 - protein, 99 aa, Artificial Sequence
  Description of Artificial Sequence Synthetic polypeptide
MADINVQVTRNNNSDSWSDIYYAYTVTTESELQKVLNELKDYIKSLLSSYALVYISITARTKKEAEQFAAILIKVFADLGPGAIDVTFTGDTVTVEGSL

SEQ ID NO: 316 - protein, 99 aa, Artificial Sequence
  Description of Artificial Sequence Synthetic polypeptide
MADINVQVTRGLSSAYLYDYYYAYTVTTESELQKVLNELKDYIKSDGDDYSNVRISITARTKKEAEQFAAILIKVFADLGWYPIDVTFTGDTVTVEGSL

SEQ ID NO: 317 - protein, 99 aa, Artificial Sequence
  Description of Artificial Sequence Synthetic polypeptide
MADINVQVTRGLSSAYAYDYYYAYTVTTESELQKVLNELKDYIKSLASSYALVYISITARTKKEAEQFAAILIKVFADLGPGAIDVTFTGDTVTVEGSL

SEQ ID NO: 318 - protein, 99 aa, Artificial Sequence
  Description of Artificial Sequence Synthetic polypeptide
MADINVQVTRGASSAYAYDYYYAYTVTTESELQKVLNELKDYIKSLASSYAAVYISITARTKKEAEQFAAILIKVFADLGPGAIDVTFTGDTVTVEGSL

SEQ ID NO: 319 - protein, 99 aa, Artificial Sequence
  Description of Artificial Sequence Synthetic polypeptide
MADINVQVTRGLSSAALYDYYYAYTVTTESELQKVLNELKDYIKSLLSSAALVYISITARTKKEAEQFAAILIKVFADLGPGAIDVTFTGDTVTVEGSL

SEQ ID NO: 320 - protein, 99 aa, Artificial Sequence
  Description of Artificial Sequence Synthetic polypeptide
MADINVQVTRGLSSAALADYYYAYTVTTESELQKVLNELKDYIKSLLSSAALVAISITARTKKEAEQFAAILIKVFADLGPGAIDVTFTGDTVTVEGSL

SEQ ID NO: 321 - protein, 99 aa, Artificial Sequence
  Description of Artificial Sequence Synthetic polypeptide
MADINVQVTRGASSAALADYYYAYTVTTESELQKVLNELKDYIKSLASSAAAVYISITARTKKEAEQFAAILIKVFADLGPGAIDVTFTGDTVTVEGSL

SEQ ID NO: 322 - protein, 99 aa, Artificial Sequence
  Description of Artificial Sequence Synthetic polypeptide
MADINVQVTIGLSSAYLYDYYYAYTVTTESELQKVLNELKDYIKSLLSSYALVYISITARTKKEAEQFAAILIKVFADLGPGAIDVTFTGDTVTVEGSL

SEQ ID NO: 323 - protein, 97 aa, Artificial Sequence
  Description of Artificial Sequence Synthetic polypeptide
MADINVQVTRGLSSYYDYYYAYTVTTESELQKVLNELKDYIKSLLSSYALVYISITARTKKEAEQFAAILIKVFADLGPGAIDVTFTGDTVTVEGSL

SEQ ID NO: 324 - protein, 97 aa, Artificial Sequence
  Description of Artificial Sequence Synthetic polypeptide
MADINVQVTRGLSSAYLYDYYYAYTVTTESELQKVLNELKDYIKSLSYALVYISITARTKKEAEQFAAILIKVFADLGPGAIDVTFTGDTVTVEGSL

SEQ ID NO: 325 - protein, 99 aa, Artificial Sequence
  Description of Artificial Sequence Synthetic polypeptide
MADINVQVTRSLYAYLGSDYYYAYTVTTESELQKVLNELKDYIKLSLASYLYVSISITARTKKEAEQFAAILIKVFADLGGAPIDVTFTGDTVTVEGSL

SEQ ID NO: 326 - protein, 99 aa, Artificial Sequence
  Description of Artificial Sequence Synthetic polypeptide
MADINVQVTRNSWDNNSIDSYYAYTVTTESELQKVLNELKDYIKDNRDGSDYVSISITARTKKEAEQFAAILIKVFADLGPYWIDVTFTGDTVTVEGSL

SEQ ID NO: 327 - protein, 96 aa, Artificial Sequence
  Description of Artificial Sequence Synthetic polypeptide
MADINVQVTRDDNGKNFDYAYTVTTESELQKVLNELKDYIKSLLSSYALVYISITARTKKEAEQFAAILIKVFADLGPGAIDVTFTGDTVTVEGSL

SEQ ID NO: 328 - protein, 97 aa, Artificial Sequence
  Description of Artificial Sequence Synthetic polypeptide
MADINVQVTRGLSSAYLYDYYYAYTVTTESELQKVLNELKDYIKKQGAKRVRISITARTKKEAEQFAAILIKVFADLGPGAIDVTFTGDTVTVEGSL

SEQ ID NO: 329 - protein, 99 aa, Artificial Sequence
  Description of Artificial Sequence Synthetic polypeptide
MADINVQVTRGLSSAYLYDYYYAYTVTTESELQKVLNELKDYIKSLLSSYALVYISITARTKKEAEQFAAILIKVFADLGYNDIDVTFTGDTVTVEGQL

SEQ ID NO: 330 - protein, 94 aa, Artificial Sequence; identical to SEQ ID NO: 312
  Description of Artificial Sequence Synthetic polypeptide

SEQ ID NO: 331 - protein, 97 aa, Artificial Sequence
  Description of Artificial Sequence Synthetic polypeptide
MADINVQVNIDDNGKNFDYAYTVGGGGGGGGLQKVLNELKDYIKKQGAKRVRISITARTKKEAEQFAAILIKVFADLGYNDIDVTFTGDTVTVEGQL

SEQ ID NO: 332 - protein, 97 aa, Artificial Sequence
  Description of Artificial Sequence Synthetic polypeptide
MADINVQVNIDDNGKNFDYAYTVTTESELQKVLNELKDYIKKQGAKRVRISITAGGGGGGGGAEQFAAILIKVFADLGYNDIDVTFTGDTVTVEGQL

SEQ ID NO: 333 - protein, 96 aa, Artificial Sequence
  Description of Artificial Sequence Synthetic polypeptide
MADINVQVNIDDNGKNFDYAYTVTTESELQKVLNELKDYIKKQGAKRVRISITAGGGGGGGGGGFAAILIKVFADLGYNDIDVTFTGDTVTVEGQL

SEQ ID NO: 334 - protein, 98 aa, Artificial Sequence
  Description of Artificial Sequence Synthetic polypeptide
MADINVQVNIDDNGKNFDYAYTVTTESELQKVLNELKDYIKKQGAKRVRISITARTKKEAEQFAAILIKVFADLGYNDIDVTFGGGGGGGGVTVEGQL

SEQ ID NO: 335 - protein, 99 aa, Artificial Sequence
  Description of Artificial Sequence Synthetic polypeptide
MADINVQVNIDDNGKNFDYAYTVGGGGGGGGLQKVLNELKDYIKKQGAKRVRISITAGGGGGGGGGGFAAILIKVFADLGYNDIDVTFTGDTVTVEGQL

SEQ ID NO: 336 - protein, 103 aa, Artificial Sequence
  Description of Artificial Sequence Synthetic polypeptide
MADINVQVNIDDNGKNFDYAYTVGGGGGGGGLQKVLNELKDYIKKQGAKRVRISITAGGGGGGGGGGFAAILIKVFADLGYNDIDVTFGGGGGGGGVTVEGQL

SEQ ID NO: 337 - protein, 97 aa, Artificial Sequence
  Description of Artificial Sequence Synthetic polypeptide
MADINVQVNIDDNGKNFDYAYNVNNSDSWSILQKVLNELKDYIKKQGAKRVRISITARTKKEAEQFAAILIKVFADLGYNDIDVTFTGDTVTVEGQL

SEQ ID NO: 338 - protein, 95 aa, Artificial Sequence
  Description of Artificial Sequence Synthetic polypeptide
MADINVQVNIDDNGKNFDYAYTVTTESELQKVLNELKDYIKKQGAKRVRISITASDGDDYSNRFAAILIKVFADLGYNDIDVTFTGDTVTVEGQL

SEQ ID NO: 339 - protein, 95 aa, Artificial Sequence
  Description of Artificial Sequence Synthetic polypeptide
MADINVQVNIDDNGKNFDYAYTVTTESELQKVLNELKDYIKKQGAKRVRISIVASDGDDYSNRFAAILIKVFADLGYNDIDVTFTGDTVTVEGQL

SEQ ID NO: 340 - protein, 94 aa, Artificial Sequence
  Description of Artificial Sequence Synthetic polypeptide
MADINVQVNIDDNGKNFDYAYTVTTESELQKVLNELKDYIKKQGAKRVRISITARTKKEAEQFAAILIKVFADLGYNDIDVTFWGPYVTVEGQL

SEQ ID NO: 341 - protein, 97 aa, Artificial Sequence
  Description of Artificial Sequence Synthetic polypeptide
MADINVQVNIDDNGKNFDYAYNVNNSDSWSILQKVLNELKDYIKKQGAKRVRISITARTKKEAEQFAAILIKVFADLGYNDIDVTFWGPYVTVEGQL

SEQ ID NO: 342 - protein, 95 aa, Artificial Sequence
  Description of Artificial Sequence Synthetic polypeptide
MADINVQVNIDDNGKNFDYAYTVTTESELQKVLNELKDYIKKQGAKRVRISITASDGDDYSNRFAAILIKVFADLGYNDIDVTFWGPYVTVEGQL

SEQ ID NO: 343 - protein, 95 aa, Artificial Sequence
  Description of Artificial Sequence Synthetic polypeptide
MADINVQVNIDDNGKNFDYAYTVTTESELQKVLNELKDYIKKQGAKRVRISIVASDGDDYSNRFAAILIKVFADLGYNDIDVTFWGPYVTVEGQL

SEQ ID NO: 344 - protein, 98 aa, Artificial Sequence
  Description of Artificial Sequence Synthetic polypeptide
MADINVQVNIDDNGKNFDYAYNVNNSDSWSILQKVLNELKDYIKKQGAKRVRISIVASDGDDYSNRFAAILIKVFADLGYNDIDVTFWGPYVTVEGQL

SEQ ID NO: 345 - protein, 116 aa, Artificial Sequence
  Description of Artificial Sequence Synthetic polypeptide
MADINVQVNIDDGGGGGGGGNFDYAYTVGGGGGGGGLQKVLNELKDYIKGGGGGGGGRVRISITAGGGGGGGGGGFAAILIKVFADLGGGGGGGGDIDVTFGGGGGGGGVTVEGQL

SEQ ID NO: 346 - protein, 101 aa, Artificial Sequence
  Description of Artificial Sequence Synthetic polypeptide
MADINVQVTRDPRRPGGDYYAYNVNNSDSWSILQKVLNELKDYIKPSSILPYYVSISIVASDGDDYSNRFAAILIKVFADLGPYSIDVTFWGPYVTVEGNY

(19) United States
(12) Patent Application Publication
(10) Pub. No.: US 2010/0029499 A1
(43) Pub. Date: Feb. 4, 2010

(54) Artificial Protein Scaffolds

(75) Inventor: Jonathan H. Davis, Auburndale, MA (US)

Correspondence Address: GOODWIN PROCTER LLP;PATENT ADMINISTRATOR, 53 STATE STREET, EXCHANGE PLACE, BOSTON, MA, 02109-2881, US

(73) Assignee: Merck Patent GmbH, Darmstadt (DE)

(21) Appl. No.: 12/429,930

(22) Filed: Apr. 24, 2009

Related U.S. Application Data
(60) Provisional application No. 61/048,099, filed on Apr. 25, 2008.

(51) Int. Cl. C40B 30/04; C07K 14/00; C40B 40/10; C40B 40/06; C07H 21/04; C12N 1/20

(52) U.S. Cl. 506/9; 530/324; 506/18; 506/16; 536/23.1; 435/252.3

(57) ABSTRACT

The present invention provides proteins having one or more similarities to the artificial protein Top7 or to a Top7 derivative. Proteins of the invention have one or more loops that are longer than the corresponding loops of Top7, and/or that bind to a preselected target molecule. The invention also provides nucleic acids and cells useful in producing the proteins and methods for their use.

### REFERENCE TO RELATED APPLICATIONS

This application claims the benefit of and priority to U.S. Provisional Patent Application No. 61/048,099, filed Apr. 25, 2008, the disclosure of which is incorporated by reference herein.

### REFERENCE TO SEQUENCE LISTING

Submitted herewith for filing is a Sequence Listing text file named LEX043.TXT. The text file is 311 kilobytes and was created on Apr. 21, 2009. The entire disclosure of the Sequence Listing text file is incorporated by reference for all purposes.

### FIELD OF THE INVENTION

This invention relates generally to artificial protein scaffolds and their design, production and use.

### BACKGROUND

Nature has provided a number of proteins into which short peptides of diverse sequences may be inserted. Antibodies are a well-known example and have antigen binding domains defined by heavy and light chain variable regions, wherein each variable region includes complementarity determining regions (CDRs) interposed between framework regions (FRs). The CDR3 loops of both heavy and light antibody chains are formed by a process in which an exonuclease and terminal transferase operate to insert an essentially random DNA sequence into each V gene that encodes a peptide loop. When this process is combined with the more limited diversity that exists in the CDR1 and CDR2 loops, the VH and VL domains are randomly paired to produce a very large number of specific protein sequences. The resulting native proteins exhibit a very large diversity of binding specificities. The so-called FRs of the antibody V domains effectively serve as a scaffold onto which the CDR loops are fused.

However, antibodies have a number of technical issues that must be addressed. For example, they generally must be produced in mammalian cells, which is expensive and time-consuming. In addition, the various methods for generating monoclonal antibodies are generally slow, expensive, or both. As a result of these problems, various groups have explored alternative protein scaffolds for the display of peptides. For example, LaVallie et al. ((1993) Biotechnology 11:187-93; and U.S. Pat. No. 5,270,181) used E. coli thioredoxin to display peptides in E. coli in a way that avoided formation of inclusion bodies. Colas et al. ((1996) Nature 380:548-50) extended this approach by showing that random peptides could be inserted into a natural loop in thioredoxin, and thioredoxin-peptide ‘aptamers’ could be selected by their binding specificities to various proteins. Other groups have identified other natural proteins that may be used as scaffolds. However, these approaches have certain limitations. In general, a scaffold based on a naturally occurring protein is best expressed in the system that normally normally produces the natural protein. For example, thioredoxin-based aptamers are generally expressed in E. coli. Conversely, fibronectin type III-based aptamers are generally best expressed in mammalian cells and/or using a secretory system that promotes disulfide bond formation. In addition, the use of naturally occurring proteins as scaffolds always has the inherent risk that an unknown biological feature of the natural protein will interfere with its function as a scaffold in a particular context. Therefore, there is a need in the art for protein scaffold systems with improved properties.

### SUMMARY OF THE INVENTION

The invention is based, in part, on the insight that a completely artificial protein, designed de novo, can have properties designated by the protein engineer, based on the needs of its intended use. At the center of the invention are artificial proteins incorporating or mimicking elements of the Top7 protein, a highly stable protein designed de novo by Kuhlmann et al. (2003) Science 302:1364-1368. These artificial proteins are designed to be highly stable and fold efficiently, with certain positions at which random or diverse peptide loops can be genetically incorporated. The stability of these artificial protein scaffolds allows the incorporation of peptides that might tend to destabilize the protein, allowing protein folding in spite of the presence of what may be destabilizing loops. If randomized amino acid sequences are introduced, the resulting protein library can be screened for the ability to bind a preselected target molecule. Proteins that result from such a screen can be used in diagnostics and therapeutics.

Accordingly, in one aspect, the invention provides a protein having a Top7 fold. One or more loops in the Top7 fold bind specifically to a preselected target molecule, to which the protein binds with a dissociation constant of no more than 10 μM (e.g. 5-10 μM, 1-10 μM, 0.5-10 μM, 0.1-10 μM, 0.05-10 μM, 0.01-10 μM, 0.001-10 μM, etc.).

In another aspect, the invention provides a protein having a Top7 fold defining two ends. At least two loops on one end of the protein are each at least one amino acid longer than the corresponding loops of Top7. In one embodiment, one or both of the two loops bind specifically to a preselected target molecule. In certain embodiments, the protein binds the preselected target molecule with a dissociation constant of no more than 10 μM (e.g. 5-10 μM, 1-10 μM, 0.5-10 μM, 0.1-10 μM, 0.05-10 μM, 0.01-10 μM, 0.001-10 μM, etc.).

In another aspect, the invention provides a protein including at least five antiparallel β-strands, at least two parallel α-helices, and loops connecting the α-helices and β-strands. Generally, the parallel α-helices form one layer and the antiparallel β-strands form a second layer. The protein has two ends, generally corresponding to the ends of the α-helices and β-strands. Each of the two ends of the protein includes two loops connecting an α-helix with a β-strand and one loop connecting two β-strands. At least two loops on one end of the protein are each at least one amino acid longer than the corresponding loops of Top7. In some embodiments, the α-helices and β-strands define an α-carbon backbone having a structure whose root mean square deviation (RMSD) from the structure of the α-carbon backbone of the α-helices and β-strands of Top7 is no greater than 4.0 (e.g. no greater than 3.5, no greater than 3.0, no greater than 2.5, no greater than 2.0, no greater than 1.9, no greater than 1.8, no greater than 1.7, no greater than 1.6, no greater than 1.5, no greater than 1.4, no greater than 1.3, no greater than 1.2, no greater than 1.1, or no greater than 1.0). In certain embodiments, at least one of the two loops binds specifically to a preselected target molecule. For example, in some embodiments the protein binds a preselected target molecule with a dissociation constant of no more than 10 μM (e.g. 5-10 μM, 1-10 μM, 0.5-10 μM, 0.1-10 μM, 0.05-10 μM, 0.01-10 μM, 0.001-10 μM, etc.).

In another aspect, the invention provides a protein including at least five antiparallel β-strands and at least two parallel α-helices, the α-helices and β-strands define an α-carbon backbone having a structure whose root mean square deviation (RMSD) from the structure of the α-carbon backbone of the α-helices and β-strands of Top7 is no greater than 4.0 (e.g. no greater than 3.5, no greater than 3.0, no greater than 2.5, no greater than 2.0, no greater than 1.9, no greater than 1.8, no greater than 1.7, no greater than 1.6, no greater than 1.5, no greater than 1.4, no greater than 1.3, no greater than 1.2, no greater than 1.1, or no greater than 1.0). The protein includes loops connecting the α-helices and β-strands. Each of two ends of the protein includes two loops connecting an α-helix with a β-strand and one loop connecting two β-strands. One or more of the loops on one end bind specifically to a preselected target molecule to which the protein binds with a dissociation constant of no more than 10 μM (e.g. 5-10 μM, 1-10 μM, 0.5-10 μM, 0.1-10 μM, 0.05-10 μM, 0.01-10 μM, 0.001-10 μM, etc.). In some embodiments, the parallel α-helices (“α”) and the antiparallel β-strands (“β”) are present in a single polypeptide, in the order ββαβαββ. In other embodiments, the protein includes two polypeptides, e.g. as a heterodimer or homodimer, each polypeptide including an α-helix and three antiparallel β-strands in the order βαββ.

In some embodiments of any one of the previously described proteins, at least three loops (e.g. three loops on the same end of the protein) are each at least one amino acid longer than the corresponding loop of Top7.

The invention also provides proteins including amino acid sequences related to an amino acid sequence of Top7 or of a Top7 derivative. The amino acid sequence of one such derivative, referred to herein as “RD1.3/1.4 Consensus,” is presented as SEQ ID NO:5. Selected amino acids from portions of the α-helices and β-strands of RD 1.3/1.4 Consensus have been concatenated and presented as SEQ ID NO:6. The amino acid sequence of another Top7 derivative, referred to as “RD1-DI-DeLys,” is presented as SEQ ID NO:2, and selected portions from its α-helices and β-strands have been concatenated and presented as SEQ ID NO:3. A concatenation of corresponding selected portions of a further consensus sequence embracing various Top7 derivatives predicted to demonstrate reduced immunogenicity is presented as SEQ ID NO:7. Specifically, for each of SEQ ID NO:3, SEQ ID NO:6, and SEQ ID NO:7, amino acids 1-5 correspond to a portion of the first β-strand; amino acids 6-8 correspond to a portion of the second β-strand; amino acids 9-20 correspond to a portion of the first α-helix; amino acids 21-23 correspond to a portion of the third β-strand; amino acids 24-32 correspond to a portion of the second α-helix; amino acids 33-37 correspond to a portion of the fourth β-strand; and amino acids 38-42 correspond to a portion of the fifth β-strand.

Accordingly, in one aspect, the invention provides a protein including an amino acid sequence of the formula B(4)-L(45)-A(5)-L(56)-B(6)-L(67)-B(7). B(4), A(5), B(6), and B(7) correspond either to (i) amino acids 21-23, 24-32, 33-37, and 38-42 of SEQ ID NO:3 or a sequence at least 80% identical to amino acids 21-42 of SEQ ID NO:3 (e.g. differing from amino acids 21-42 at no more than four positions, no more than three positions, no more than two positions, or no more than one position); or (ii) amino acids 21-23, 24-32, 33-37, and 38-42 of SEQ ID NO:6 or a sequence at least 90% identical to amino acids 21-42 of SEQ ID NO:6 (e.g. differing from amino acids 21-42 at no more than two positions or no more than one position); or (iii) amino acids 21-23, 24-32, 33-37, and 38-42 of SEQ ID NO:7 or a sequence at least 95% identical to amino acids 21-42 of SEQ ID NO:7. The minimum lengths of L(45), L(56), and L(67) are 10 amino acids, 7 amino acids, and 4 amino acids, respectively. At least one of L(45), L(56), and L(67) specifically binds a preselected target molecule, to which the protein binds with an affinity constant of no more than 10 μM.

In another aspect, the invention provides a protein including an amino acid sequence of the formula B(4)-L(45)-A(5)-L(56)-B(6)-L(67)-B(7). B(4), A(5), B(6), and B(7) correspond either to (i) amino acids 21-23, 24-32, 33-37, and 38-42 of SEQ ID NO:3 or a sequence at least 80% identical to amino acids 21-42 of SEQ ID NO:3 (e.g. differing from amino acids 21-42 at no more than four positions, no more than three positions, no more than two positions, or no more than one position); or (ii) amino acids 21-23, 24-32, 33-37, and 38-42 of SEQ ID NO:6 or a sequence at least 90% identical to amino acids 21-42 of SEQ ID NO:6 (e.g. differing from amino acids 21-42 at no more than two positions or no more than one position); or (iii) amino acids 21-23, 24-32, 33-37, and 38-42 of SEQ ID NO:7 or a sequence at least 95% identical to amino acids 21-42 of SEQ ID NO:7. The minimum lengths of L(45), L(56), and L(67) are 10 amino acids, 7 amino acids, and 4 amino acids, respectively, and at least two of L(45), L(56), and L(67) each exceed their minimum length by at least one amino acid. In some embodiments, at least one of L(45), L(56), and L(67) specifically binds a preselected target molecule, to which the protein binds with an affinity constant of no more than 10 μM.

In certain embodiments, a protein of the invention includes two amino acid sequences of the formula B(4)-L(45)-A(5)-L(56)-B(6)-L(67)-B(7) (e.g. on separate polypeptide chains).

In another aspect, the invention provides a protein including an amino acid sequence of the formula B(1)-L(12)-B(2)-L(23)-A(3)-L(34)-B(4)-L(45)-A(5)-L(56)-B(6)-L(67)-B (7). B(1), B(2), A(3), B(4), A(5), B(6), and B(7) correspond either to (i) amino acids 1-5, 6-8, 9-20, 21-23, 24-32, 33-37, and 38-42 of SEQ ID NO:3 or a sequence at least 80% identical to amino acids 1-42 of SEQ ID NO:3 (e.g. differing from amino acids 1-42 at no more than eight positions, no more than seven positions, no more than six positions, no more than five positions, no more than four positions, no more than three positions, no more than two positions, or no more than one position); or (ii) amino acids 1-5, 6-8, 9-20, 21-23, 24-32, 33-37, and 38-42 of SEQ ID NO:6 or a sequence at least 90% identical to amino acids 1-42 of SEQ ID NO:6 (e.g. differing from amino acids 1-42 at no more than four positions, no more than three positions, no more than two positions or no more than one position); or (iii) amino acids 1-5, 6-8, 9-20, 21-23, 24-32, 33-37, and 38-42 of SEQ ID NO:7 or a sequence at least 95% identical to SEQ ID NO:7. The minimum lengths of L(12), L(23), L(34), L(45), L(56), and L(67) are 10 amino acids, 7 amino acids, 9 amino acids, 10 amino acids, 7 amino acids, and 4 amino acids, respectively. At least one of L(12), L(23), L(34), L(45), L(56), and L(67) specifically binds a preselected target molecule, to which the protein binds with an affinity constant of no more than 10 μM. In some embodiments, B(1), B(2), A(3), B(4), A(5), B(6), and B(7) correspond to (i) amino acids 1-5, 6-8, 9-20, 21-23, 24-32, 33-37, and 38-42 of SEQ ID NO:3 or a sequence at least 85% identical to SEQ ID NO:3; or (ii) amino acids 1-5, 6-8, 9-20, 21-23, 24-32, 33-37, and 38-42 of SEQ ID NO:6 or a sequence at least 95% identical to SEQ ID NO:6.; or (iii) amino acids 1-5, 6-8, 9-20, 21-23, 24-32, 33-37, and 38-42 of SEQ ID NO:7

In another aspect, the invention provides a protein including an amino acid sequence of the formula B(1)-L(12)-B(2)-L(23)-A(3)-L(34)-B(4)-L(45)-A(5)-L(56)-B(6)-L(67)-B (7). B(1), B(2), A(3), B(4), A(5), B(6), and B(7) correspond either to (i) amino acids 1-5, 6-8, 9-20, 21-23, 24-32, 33-37, and 38-42 of SEQ ID NO:3 or a sequence at least 80% identical to amino acids 1-42 of SEQ ID NO:3 (e.g. differing from amino acids 1-42 at no more than eight positions, no more than seven positions, no more than six positions, no more than five positions, no more than four positions, no more than three positions, no more than two positions, or no more than one position); or (ii) amino acids 1-5, 6-8, 9-20, 21-23, 24-32, 33-37, and 38-42 of SEQ ID NO:6 or a sequence at least 90% identical to amino acids 1-42 of SEQ ID NO:6 (e.g. differing from amino acids 1-42 at no more than four positions, no more than three positions, no more than two positions or no more than one position); or (iii) amino acids 1-5, 6-8, 9-20, 21-23, 24-32, 33-37, and 38-42 of SEQ ID NO:7 or a sequence at least 95% identical to amino acids 1-42 of SEQ ID NO:7. The minimum lengths of L(12), L(23), L(34), L(45), L(56), and L(67) are 10 amino acids, 7 amino acids, 9 amino acids, 10 amino acids, 7 amino acids, and 4 amino acids, respectively. In some embodiments, at least two of L(12), L(34), or L(56) each exceeds its minimum length by at least one amino acid. In some embodiments, at least two of L(23), L(45), or L(67) each exceeds its minimum length by at least one amino acid.

In another aspect, the invention provides a protein including an amino acid sequence of the formula B(1)-L(12)-B(2)-L(23)-A(3)-L(34)-B(4)-L(45)-A(5)-L(56)-B(6)-L(67)-B (7). B(1), B(2), A(3), B(4), A(5), B(6), and B(7) correspond either to (i) amino acids 1-5, 6-8, 9-20, 21-23, 24-32, 33-37, and 38-42 of SEQ ID NO:3 or a sequence at least 85% identical to amino acids 1-42 of SEQ ID NO:3 (e.g. differing from amino acids 1-42 at no more than six positions, no more than five positions, no more than four positions, no more than three positions, no more than two positions, or no more than one position); or (ii) amino acids 1-5, 6-8, 9-20, 21-23, 24-32, 33-37, and 38-42 of SEQ ID NO:6 or a sequence at least 95% identical to amino acids 1-42 of SEQ ID NO:6 (e.g. differing from amino acids 1-42 at no more than two positions or no more than one position); or (iii) amino acids 1-5, 6-8, 9-20, 21-23, 24-32, 33-37, and 38-42 of SEQ ID NO:7. The minimum lengths of L(12), L(23), L(34), L(45), L(56), and L(67) are 10 amino acids, 7 amino acids, 9 amino acids, 10 amino acids, 7 amino acids, and 4 amino acids, respectively, and L(12), L(23), L(34), L(45), L(56), or L(67) exceeds its minimum length by at least one amino acid. In some embodiments, B(1), B(2), A(3), B(4), A(5), B(6), and B(7) correspond to amino acids 1-5, 6-8, 9-20, 21-23, 24-32, 33-37, and 38-42 of SEQ ID NO:3 or a sequence at least 90% identical or at least 95% identical thereto. In some embodiments, the protein specifically binds a preselected target molecule in a manner dependent on the amino acid sequence of L(12), L(23), L(34), L(45), L(56), and/or L(67).

For any protein including an amino acid sequence of the formula (1)-L(12)-B(2)-L(23)-A(3)-L(34)-B(4)-L(45)-A(5)-L(56)-B(6)-L(67)-B(7), in some embodiments, at least two, at least three, at least four, at least five, or all six of L(12), L(23), L(34), L(45), L(56), and L(67) each exceeds its minimum length by at least one amino acid. These combinations of lengths are depicted in the following Table 1, in which “min” indicates that the length equals the minimum length and “>min” indicates that the length exceeds the minimum length by at least one amino acid.

TABLE 1

| Embodiment | L(12) | L(23) | L(34) | (L(45) | L(56) | L(67) |
|---|---|---|---|---|---|---|
| 1 | min | min | min | min | min | min |
| 2 | min | min | min | min | min | >min |
| 3 | min | min | min | min | >min | min |
| 4 | min | min | min | min | >min | >min |
| 5 | min | min | min | >min | min | min |
| 6 | min | min | min | >min | min | >min |
| 7 | min | min | min | >min | >min | min |
| 8 | min | min | min | >min | >min | >min |
| 9 | min | min | >min | min | min | min |
| 10 | min | min | >min | min | min | >min |
| 11 | min | min | >min | min | >min | min |
| 12 | min | min | >min | min | >min | >min |
| 13 | min | min | >min | >min | min | min |
| 14 | min | min | >min | >min | min | >min |
| 15 | min | min | >min | >min | >min | min |
| 16 | min | min | >min | >min | >min | >min |
| 17 | min | >min | min | min | min | min |
| 18 | min | >min | min | min | min | >min |
| 19 | min | >min | min | min | >min | min |
| 20 | min | >min | min | min | >min | >min |
| 21 | min | >min | min | >min | min | min |
| 22 | min | >min | min | >min | min | >min |
| 23 | min | >min | min | >min | >min | min |
| 24 | min | >min | min | >min | >min | >min |
| 25 | min | >min | >min | min | min | min |
| 26 | min | >min | >min | min | min | >min |
| 27 | min | >min | >min | min | >min | min |
| 28 | min | >min | >min | min | >min | >min |
| 29 | min | >min | >min | >min | min | min |
| 30 | min | >min | >min | >min | min | >min |
| 31 | min | >min | >min | >min | >min | min |
| 32 | min | >min | >min | >min | >min | >min |
| 33 | >min | min | min | min | min | min |
| 34 | >min | min | min | min | min | >min |
| 35 | >min | min | min | min | >min | min |
| 36 | >min | min | min | min | >min | >min |
| 37 | >min | min | min | >min | min | min |
| 38 | >min | min | min | >min | min | >min |
| 39 | >min | min | min | >min | >min | min |
| 40 | >min | min | min | >min | >min | >min |
| 41 | >min | min | >min | min | min | min |
| 42 | >min | min | >min | min | min | >min |
| 43 | >min | min | >min | min | >min | min |
| 44 | >min | min | >min | min | >min | >min |
| 45 | >min | min | >min | >min | min | min |
| 46 | >min | min | >min | >min | min | >min |
| 47 | >min | min | >min | >min | >min | min |
| 48 | >min | min | >min | >min | >min | >min |
| 49 | >min | >min | min | min | min | min |
| 50 | >min | >min | min | min | min | >min |
| 51 | >min | >min | min | min | >min | min |
| 52 | >min | >min | min | min | >min | >min |
| 53 | >min | >min | min | >min | min | min |
| 54 | >min | >min | min | >min | min | >min |
| 55 | >min | >min | min | >min | >min | min |
| 56 | >min | >min | min | >min | >min | >min |
| 57 | >min | >min | >min | min | min | min |
| 58 | >min | >min | >min | min | min | >min |
| 59 | >min | >min | >min | min | >min | min |
| 60 | >min | >min | >min | min | >min | >min |
| 61 | >min | >min | >min | >min | min | min |
| 62 | >min | >min | >min | >min | min | >min |
| 63 | >min | >min | >min | >min | >min | min |
| 64 | >min | >min | >min | >min | >min | >min |

For any protein of the invention, in some embodiments the protein includes an effector stably associated therewith. In this context, an “effector” provides an activity, such as a therapeutic or other biological activity. An effector can be as small as a radioisotope, useful for local delivery of (a preferably therapeutically effective dose of) radiation, or can be substantially larger, such as an organic small molecule (such as a pharmaceutical), a ligand (such as a cytokine, for example, an interleukin), a toxin (such as a chemotherapeutic agent), a binding moiety, a macrocyclic compound, an enzyme or other catalyst, a signaling protein, etc. The effector may be incorporated, e.g. as an amino acid sequence, and may be covalently connected, such as by a crosslinking moiety to an amino acid side chain or to the amino- or carboxy-terminus of the protein.

For any protein of the invention, in some embodiments the protein includes a detectable label stably associated therewith. The detectable label may be incorporated, e.g. as an amino acid sequence, and may be covalently connected, such as by a crosslinking moiety to an amino acid side chain or to the amino- or carboxy-terminus of the protein. The detectable label can include, for example, a colloidal metal (e.g. colloidal gold), a radiolabel, an epitope tag, an enzyme or other catalyst, a fluorophore, a chromophore, a quantum dot, etc.

For any protein of the invention, in some embodiments the scaffold protein of the invention includes a carrier protein stably associated therewith, e.g. as a fusion protein, or covalently associated as by a disulfide bond or a chemical crosslinker. The carrier protein can be, for example, an antibody, or a portion thereof, such as an Fc portion, an antibody variable domain, or an scFv moeity. In certain embodiments, a heterodimeric carrier protein, such as an engineered heterodimeric protein as described in U.S. Patent Application Publication US 2007/0287170, is included, permitting the association of one, two or more scaffold proteins of the invention with each other and/or with other moieties such as binding proteins, effector molecules, and/or detectable labels in a designed, engineered manner.

For any protein of the invention, in certain embodiments, the protein: does not specifically bind CD4; does not include a human immunodeficiency virus (HIV) peptide; does not include an immunogenic HIV peptide; does not include a viral peptide; does not include a bacterial peptide; and/or is not combined or co-administered with an adjuvant.

In one aspect, the invention provides a fusion protein that includes at least two of the previously described proteins.

In one aspect, the invention provides a protein library of a plurality of non-identical proteins. The non-identical proteins are as described above, but differ from each other in the amino acid sequences of one or more loops, or in at least one of L(12), L(23), L(34), L(45), L(56), or L(67). The invention also provides a nucleic acid library encoding such a protein library, as well as nucleic acids encoding any of one the proteins described above and cells containing such nucleic acids. The invention also provides methods for identifying a protein that specifically binds a preselected target molecule. The method includes exposing the protein library to a target molecule and identifying at least one protein associated with the target molecule.

In one aspect, the invention provides a method for detecting a target molecule. The method includes exposing a sample to a protein of the invention having an affinity for the target molecule under conditions permitting a target molecule, if present, to bind to the protein. The method further includes detecting the presence or absence of a complex including the protein and the target molecule.

The invention also provides a complex including a preselected target molecule and a protein of the invention having an affinity for the preselected target molecule. The protein optionally includes a detectable label, which can facilitate detection of the complex.

In one aspect, the invention provides a method of binding an in vivo target. The method includes administering a protein of the invention that specifically binds an in vivo target. In some embodiments, the protein includes a detectable label, which optionally is suitable for in vivo imaging (e.g. a radiolabel). In some embodiments the protein includes an effector, such as a therapeutic agent, a cytokine, or a toxin.

These and other aspects and advantages of the invention will become apparent upon consideration of the following figures, detailed description and claims.

### BRIEF DESCRIPTION OF THE DRAWINGS

FIG. 1 depicts the three-dimensional structure of Top7, as viewed along the axis of the first β-strand. The white arrow indicates the counterclockwise orientation of the first three structural elements of the protein, starting from the first β-strand when viewed from the N-terminus of the protein.

FIG. 2 contains the Protein Data Bank database entry (1QYS) with the atomic coordinates of the Top7 structure. The 12 mer on page 4 is disclosed as SEQ ID NO: 18; the 106 mer on page 5 is disclosed as SEQ ID NO: 19; and the peptide disclosed in atomic coordinates are residues 3-94 of SEQ ID NO: 19.

FIG. 3 depicts the arrangement of secondary structure elements, loops and ends in the Top7 structure.

FIGS. 4A and 4B depict the structures of an antibody VH domain and Top7, respectively.

FIG. 5 provides an alignment of the amino acid sequences of Top7 (SEQ ID NO: 20), RD1.3 (SEQ ID NO: 21), and RD1Lib1 (SEQ ID NO: 22).

FIG. 6 depicts an illustrative nucleic acid of the invention. 6xHis tag is disclosed as SEQ ID NO: 310 and Gly4-Ser is disclosed as SEQ ID NO: 311.

FIG. 7 depicts an illustrative method for shuffling loops among members of a library.

FIG. 8 provides an alignment of exemplary amino acid sequences of the invention (SEQ ID NOS 23-29, respectively, in order of appearance).

FIG. 9 provides additional exemplary amino acid sequences of the invention (SEQ ID NOS 21, 30, 5, 2, 21, 6, 3, 31-35, 7 and 312-346, respectively, in order of appearance).

FIGS. 10 and 11 are alignments of exemplary RD1Libl-derived proteins with an affinity for the variable domain of an antibody to the αV-chain of human αV-integrins. FIG. 10 discloses SEQ ID NOS 36-38, 38, 38-43, 43-46, 45, 47-48, 48-54, 54-67, 66, 66, 68-78 and 78-86, respectively, in order of appearance. FIG. 11 discloses SEQ ID NOS 87-110, 110-129, 129-132, 132, 132-150 and 150-152, respectively, in order of appearance.

FIG. 12 is an alignment of exemplary RD1Lib1-derived proteins with an affinity for the variable domain of antibody KS. FIG. 12 discloses SEQ ID NOS 153, 153-159, 159, 159-168, 168-171, 171, 171, 171, 171, 171-179, 179-180, 180-185, 184, 186-189 and 189-192, respectively, in order of appearance.

FIGS. 13 and 14 are alignments of exemplary RD1Lib1-derived proteins with an affinity for the variable domain of an anti-CD19 antibody. FIG. 13 discloses SEQ ID NOS 42, 193-198, 81, 78, 199, 199, 199-201, 201, 201-203, 88, 204-208, 208-209, 54, 210, 210-218, 218-219, 219, 219-225, 225, 225, 225-227, 227, 132, 228-229, 229, 229-231, 231, 231, 231-233, 233-238, 45 and 239, respectively, in order of appearance. FIG. 14 discloses SEQ ID NOS 38, 38, 38, 240, 45, 45, 45, 45, 241-259, 52, 260-261, 261-266, 80, 80, 80, 80, 267-272, 272-274, 274-275, 275-278, 278 and 278-291, respectively, in order of appearance.

FIG. 15 is an alignment of exemplary scaffold proteins bearing grafted loops from binding proteins selected from a library (SEQ ID NOS 292-297, respectively, in order of appearance).

FIG. 16 is a size exclusion chromatogram of an exemplary Fc-RDI fusion protein.

FIG. 17 is a size exclusion chromatogram of an exemplary Fc-RD1-DI-DeLys fusion protein.

FIG. 18 is a size exclusion chromatogram of an exemplary Fc-“Guy 1” fusion protein.

FIG. 19 depicts additional exemplary amino acid sequences of the invention. These include: 6-1 (SEQ ID NO: 298), a Top7 protein with a mutated glycosylation site; 6-2 through 6-4 (SEQ ID NOS 299-301, respectively, in order of appearance), slight variants of RD1.3, 6-5 through 6-9 (SEQ ID NOS 302-306, respectively, in order of appearance), RD1.3 variants with fewer immunogenic epitopes and fewer lysines; 6-10 (SEQ ID NO: 307)=an RD1 library member from Example 9; and 6-11 (SEQ ID NO: 308), a variant on the M7 protein of Dallüge et al. The consensus sequence is disclosed as SEQ ID NO: 309.

### DETAILED DESCRIPTION OF THE INVENTION

The invention is based, in part, upon the appreciation that the stability and structure of Top7-related proteins permits their use as a scaffold for the presentation of one or more heterologous amino acid sequences, which may be inserted into the scaffold and/or may replace existing amino acids of the scaffold.

### The Top7 Fold

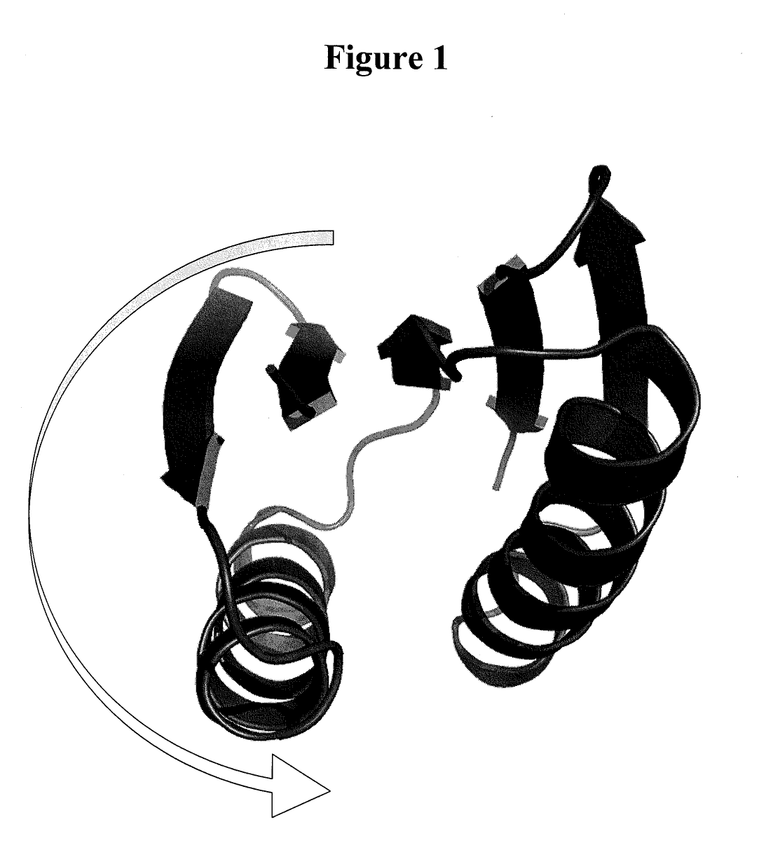

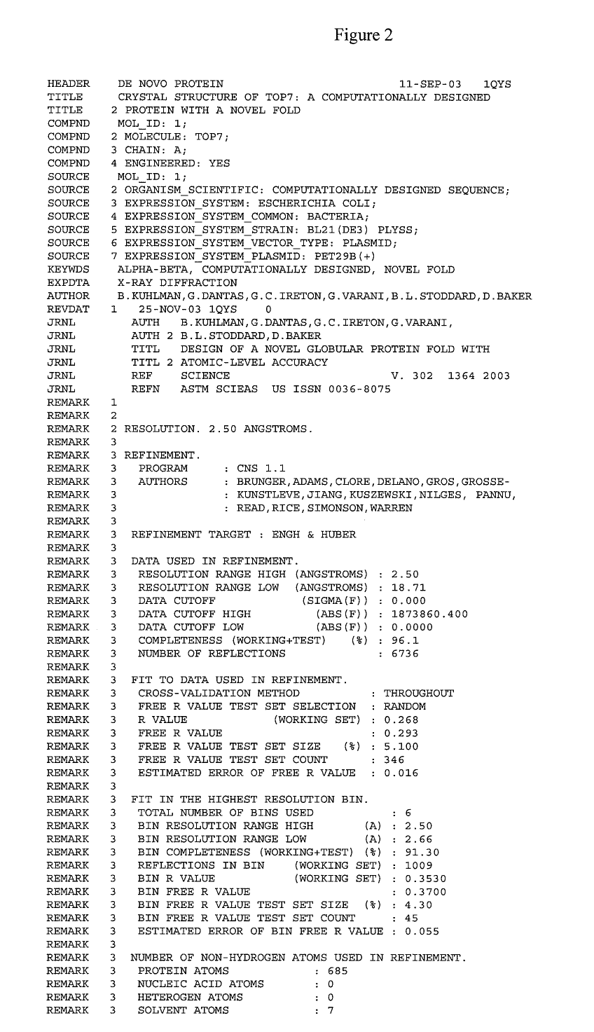

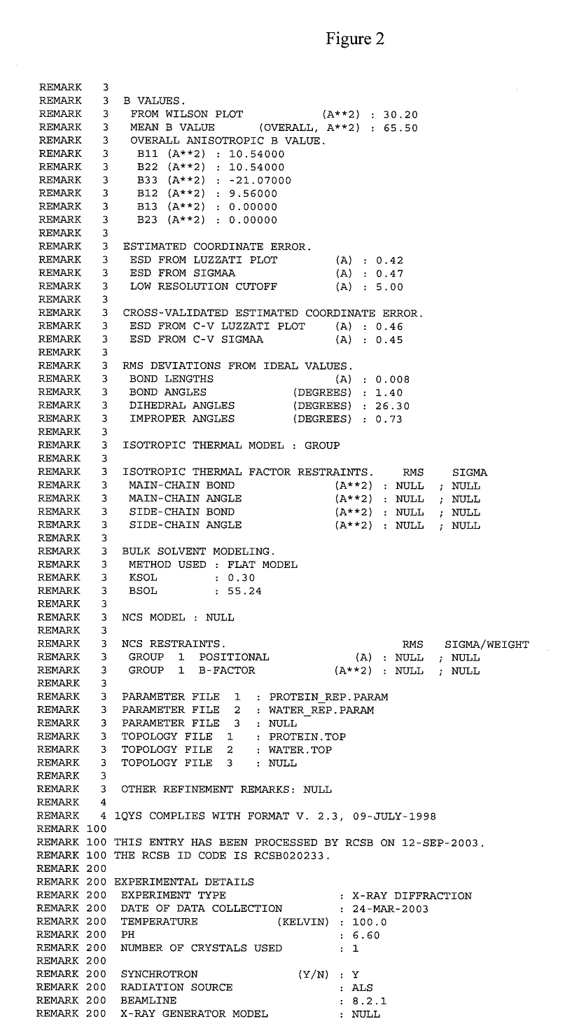

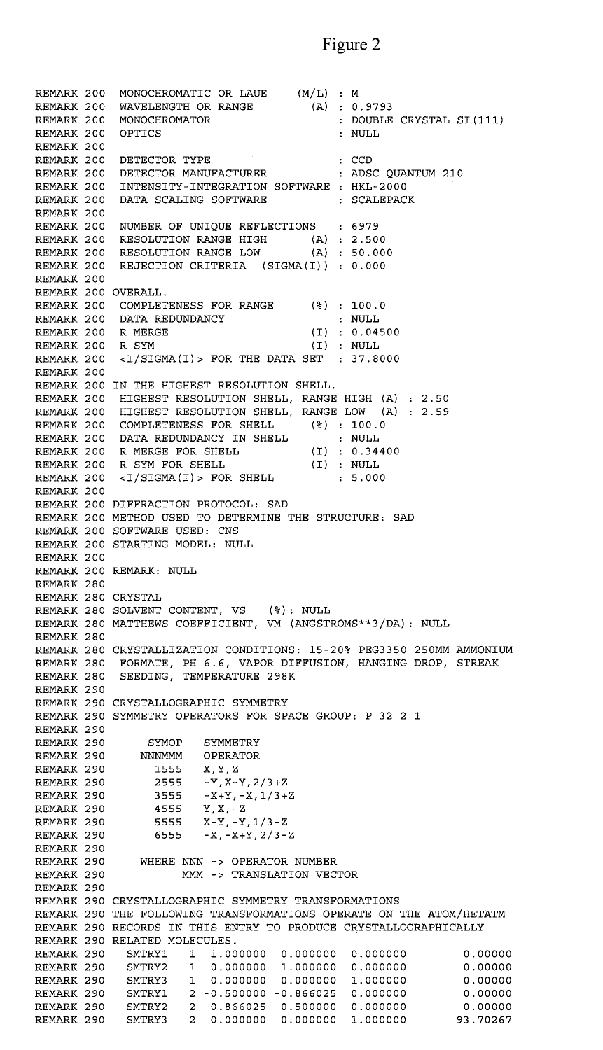

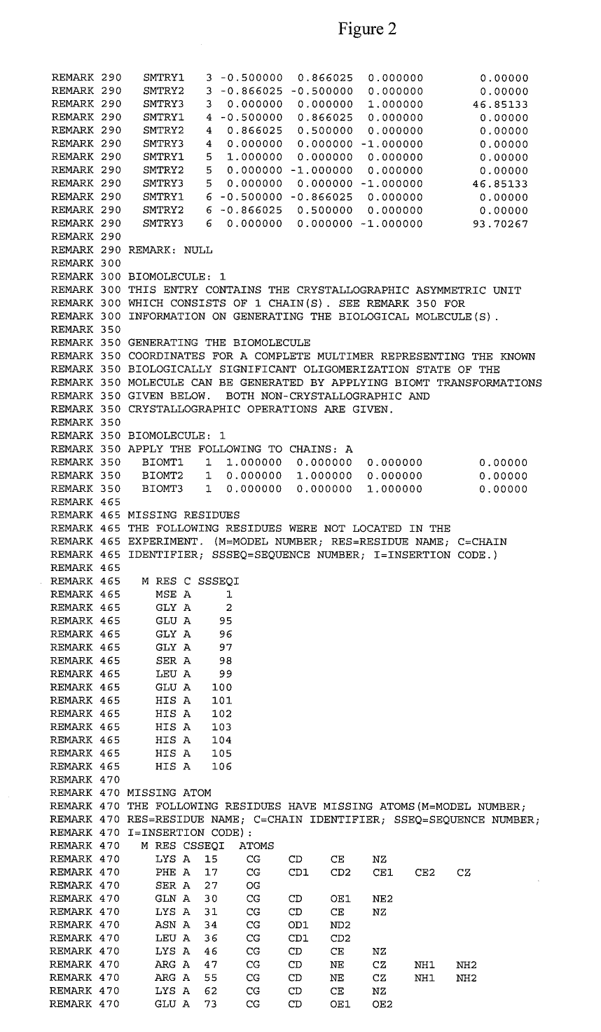

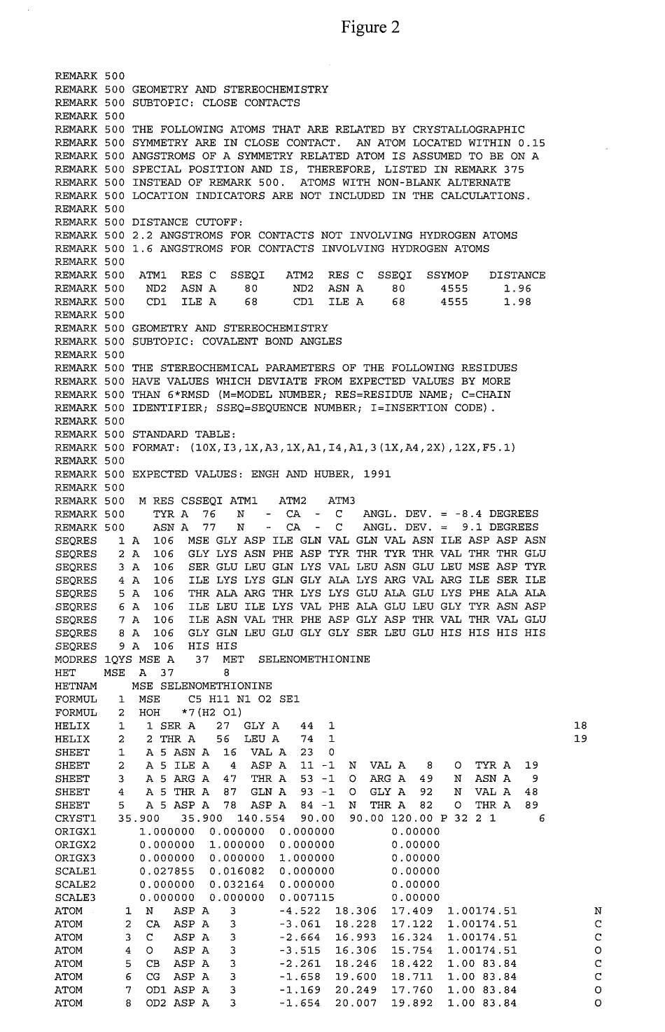

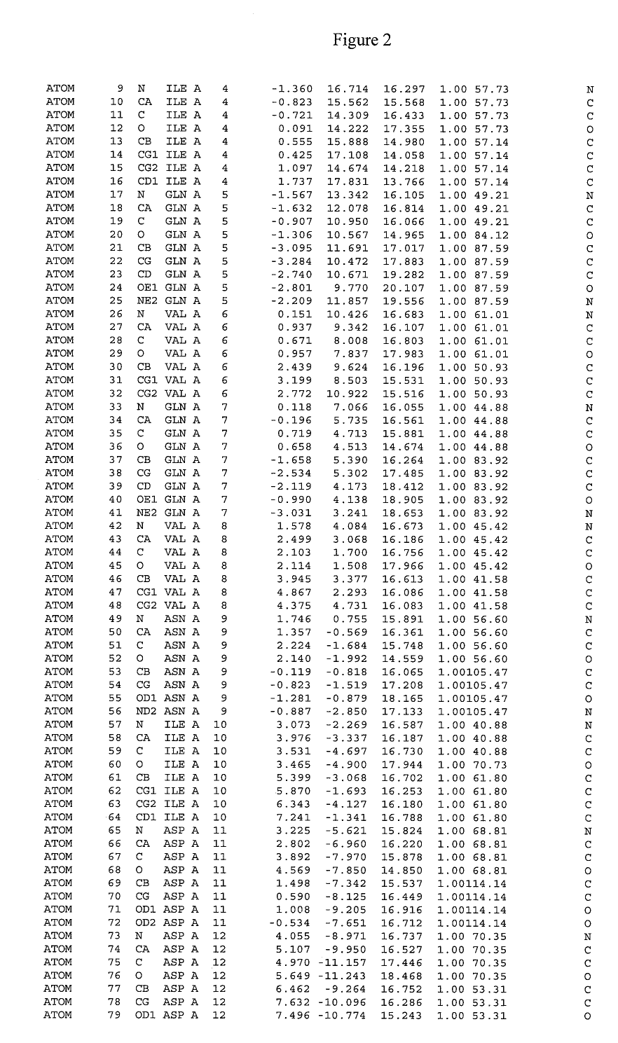

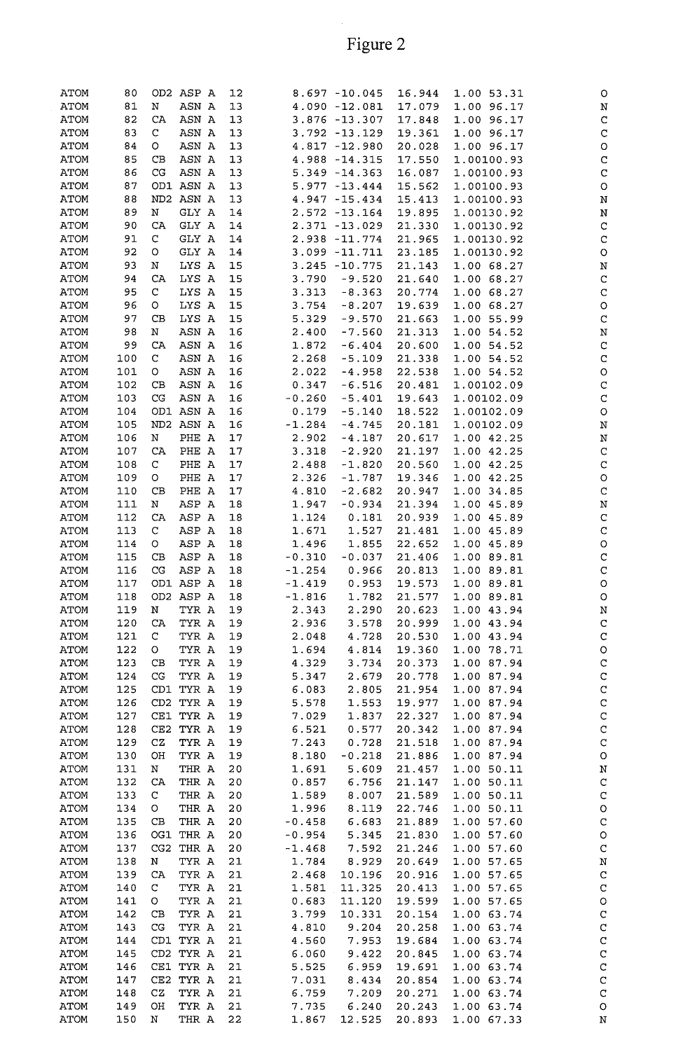

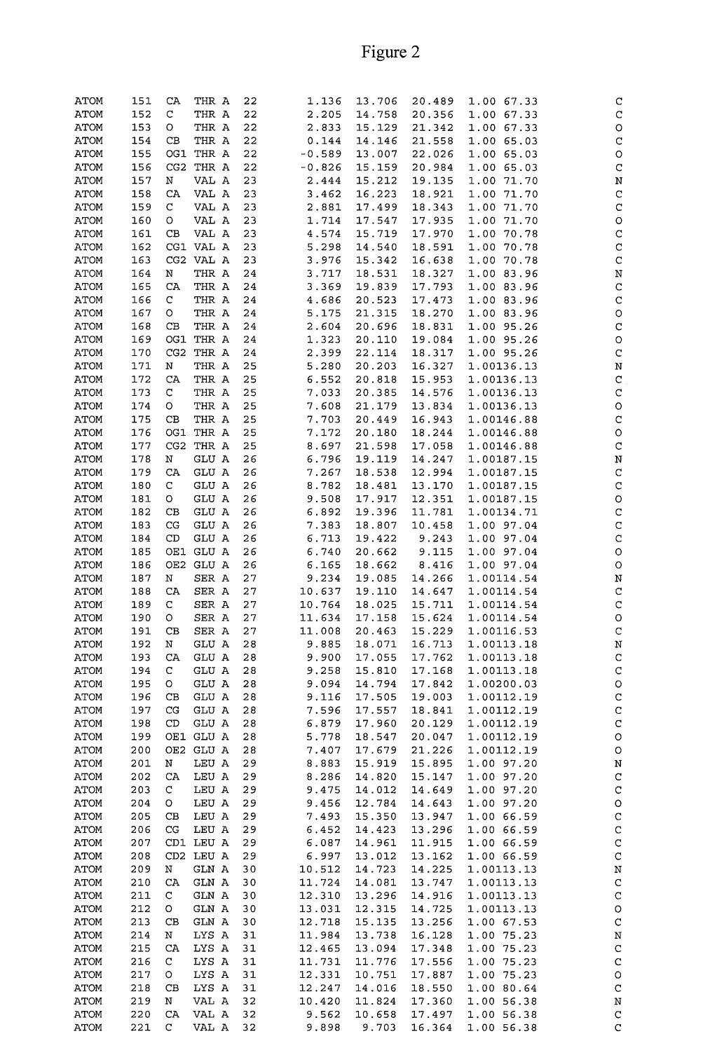

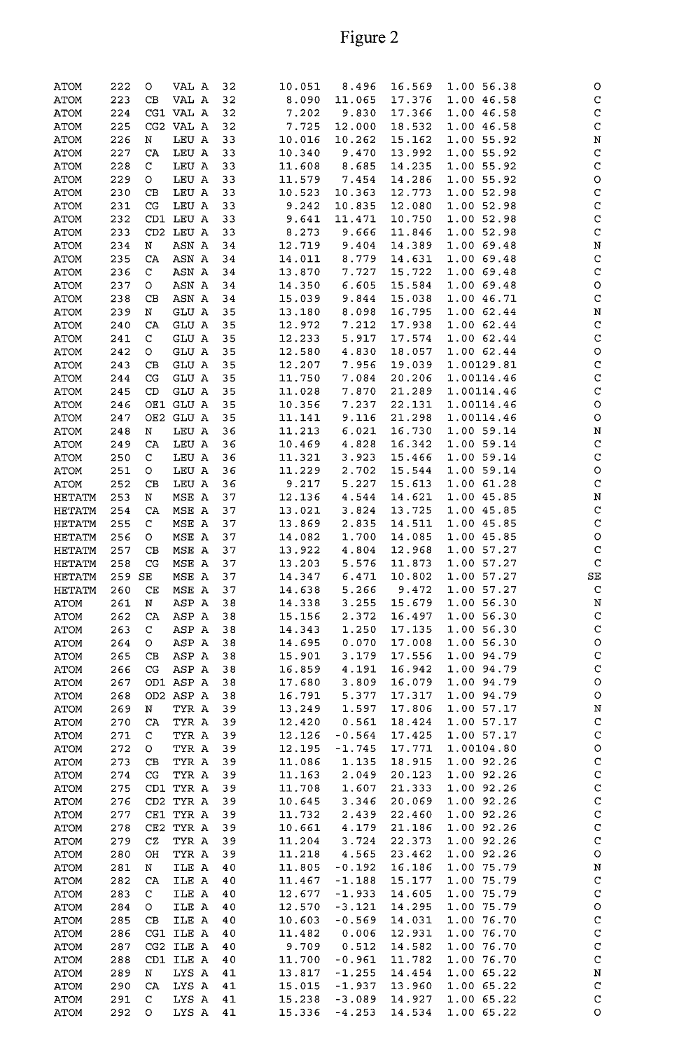

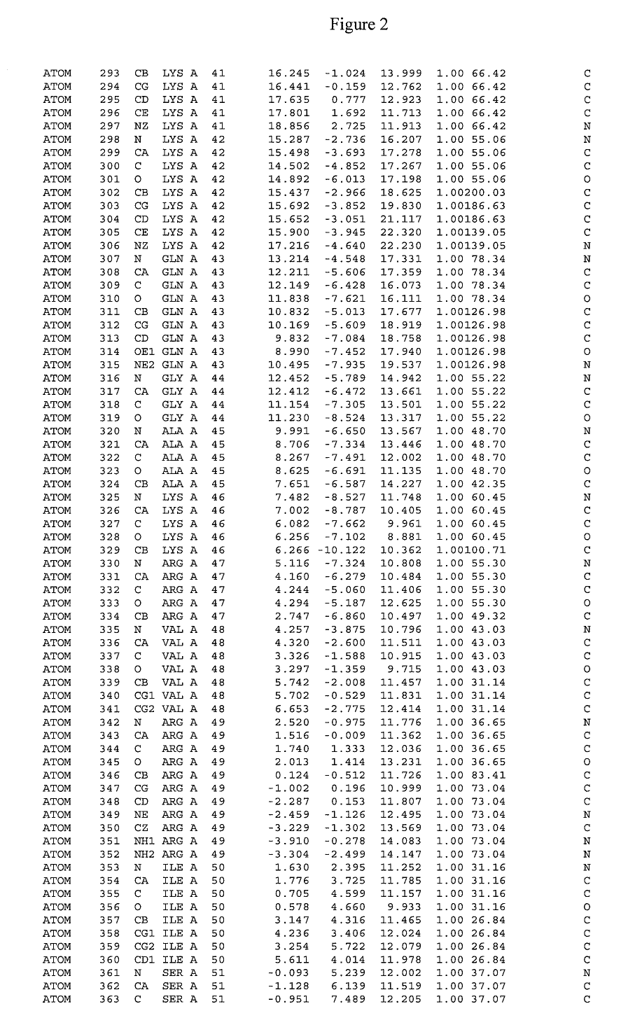

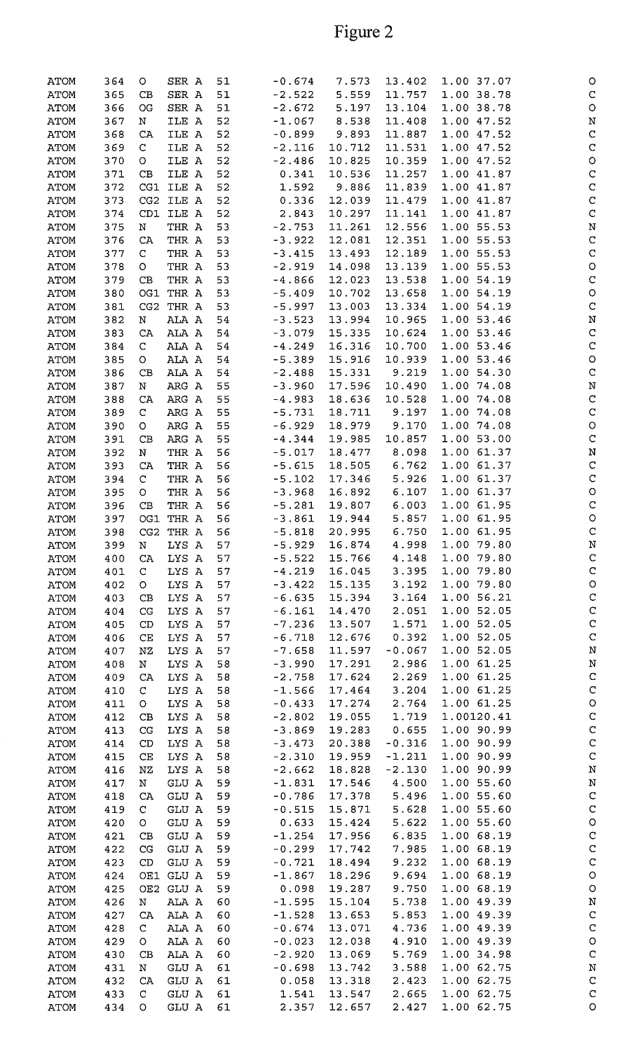

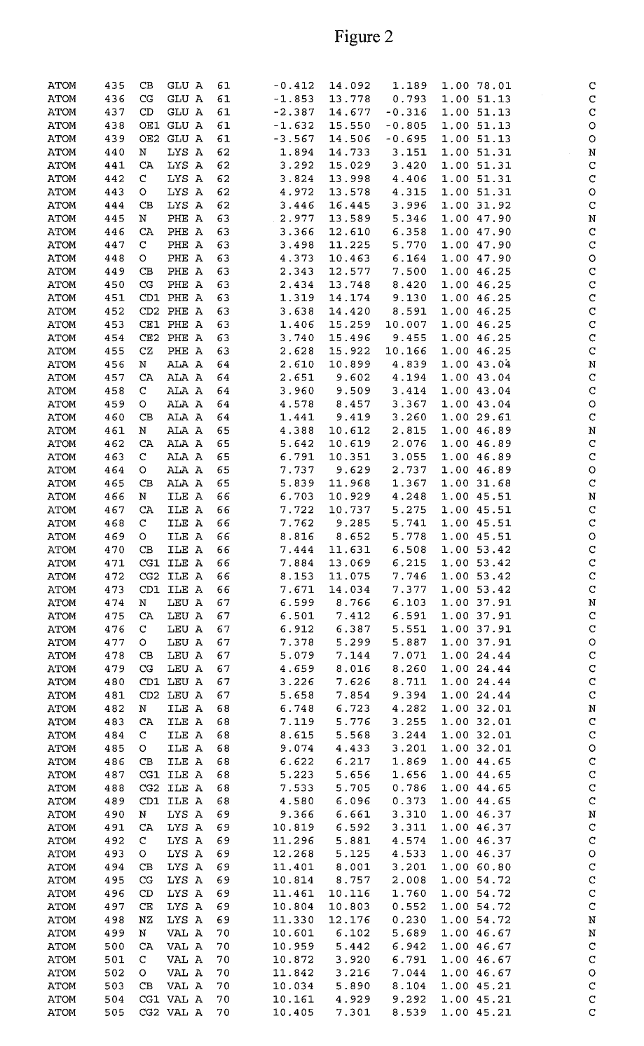

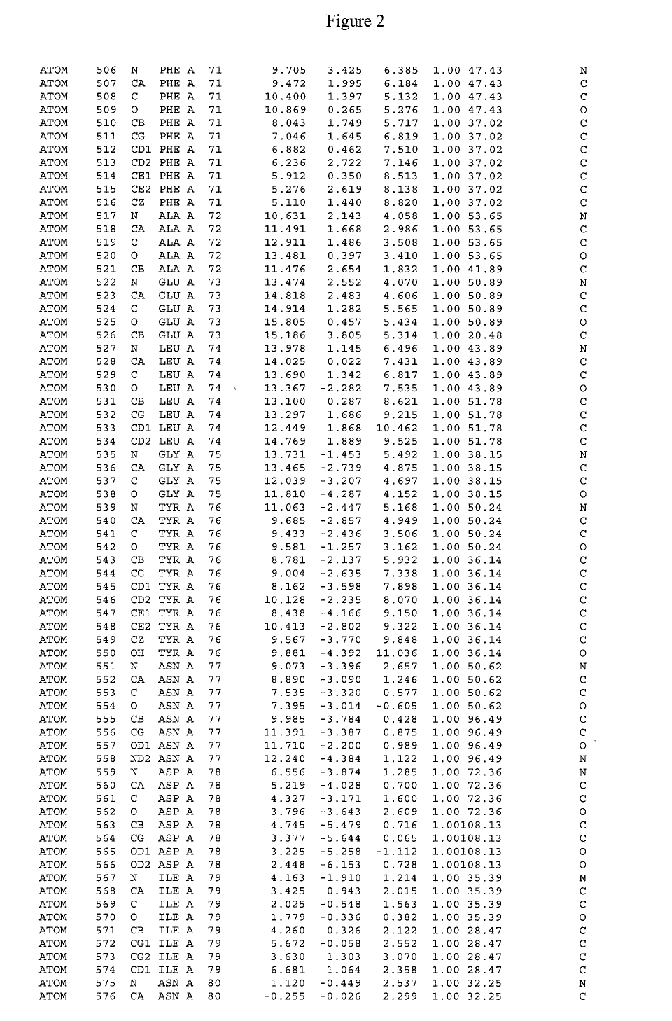

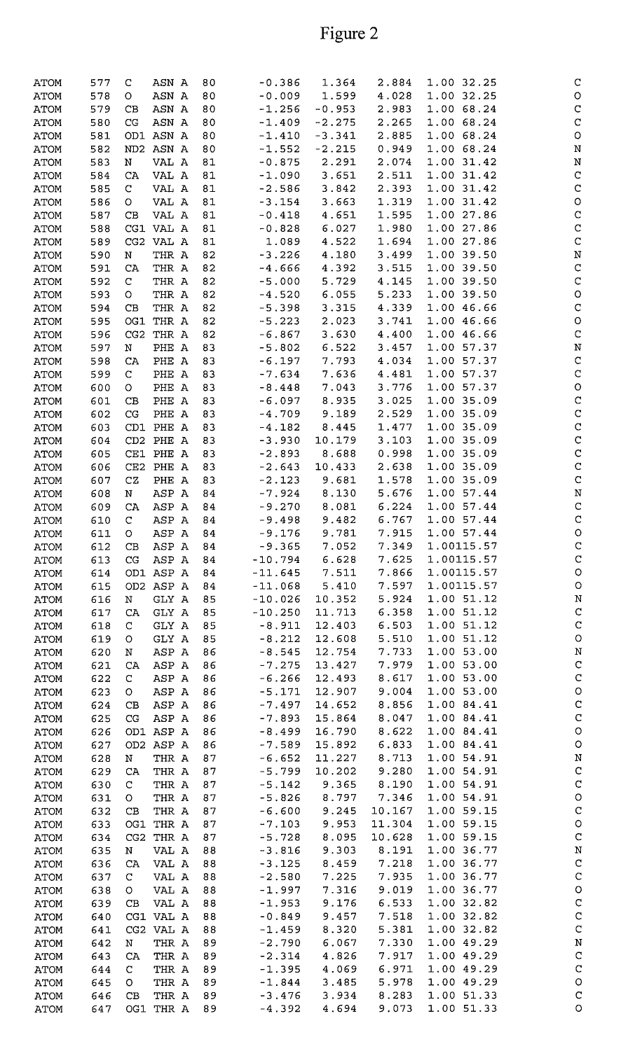

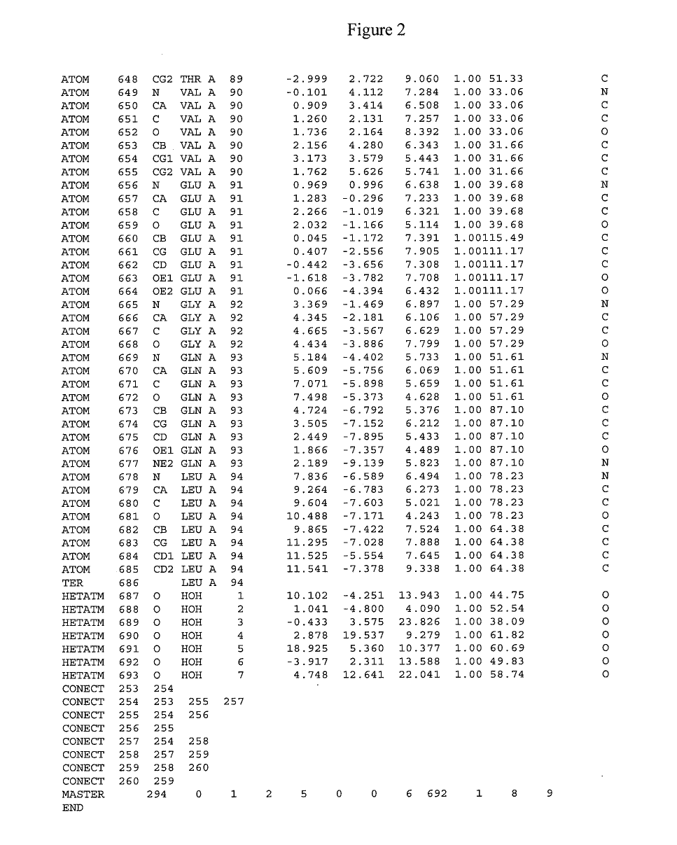

Heterologous amino acid sequences can be inserted into a protein that incorporates elements of the Top7 fold. The structure of the Top7 protein, as determined by X-ray crystallography by Kuhlman et al. ((2003) Science 302:1364-1368 and deposited in the Protein Data Base with accession number 1QYS, is shown in FIG. 1. The coordinates of the structure are also presented in FIG. 2. As seen in FIG. 1, Top7 is a two-layer protein, with two parallel α-helices on one side of the protein forming a first layer (the bottom layer in FIG. 1) packed against a second layer (the top layer in FIG. 1) formed of five antiparallel β-strands. Each secondary structure element (-60 -helix or β-strand) is directly connected to the next. In other words, none of the loops traverses the length of a structural element to connect the “near end” of one element to the “far end” of the next; rather, the loops connect the closer ends of the elements.

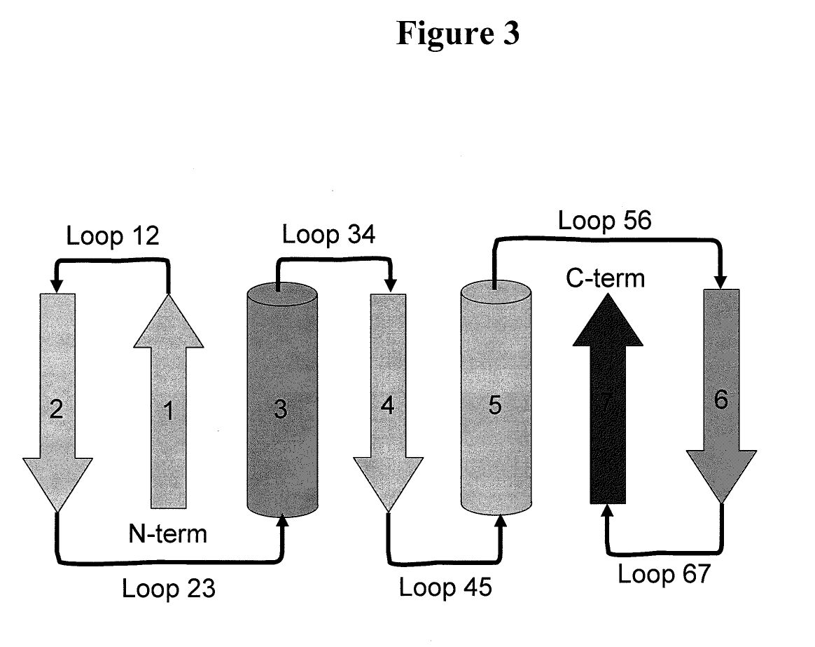

The arrangement of the secondary structure elements of Top7 in the Top7 polypeptide is shown in FIG. 3. In FIG. 3, the five β-strands are depicted as arrows and the two α-helices are depicted as cylinders. The elements are numbered sequentially from 1-7, based on the order in which they appear in the Top7 amino acid sequence. Thus, the β-strands (“β”) and α-helices (“α”) are present in the order ββαβαββ, and the first two β-strands are numbered 1 and 2; the first α-helix is numbered 3; the next β-strand is numbered 4; the second α-helix is numbered 5, and the last two β-strands are numbered 6 and 7. While the order of the elements, from the amino terminus to the carboxy terminus of Top7, is 1234567, in FIG. 3 the order of the elements from left to right is 2134576. This reflects that the β-sheet of Top7 is arranged with the second β-strand (“2”) on one side of the sheet, followed by the first β-strand (“1”), the third β-strand (structural element “4”), the fifth β-strand (structural element “7”) and, on the far end of the sheet, the fourth β-strand (structural element “6”). In FIG. 3, the loops connecting the elements are named according to the structural elements they connect. Thus, the loop connecting elements 1 and 2 is named “Loop 12,” the loop connecting elements 2 and 3 is named “Loop 23,” and so on. The end of the protein that includes loops 12, 34, and 56 is termed the “North End” and the end of the protein that includes loops 23, 45, and 67 is termed the “South End.”

In FIG. 1, the Top7 protein is oriented to provide a perspective looking from the N-terminus of the protein down the first β-strand (structural element “1”). As seen in FIG. 1, the α-helices are positioned with respect to the β-strands such that a line drawn from the first β-strand to the second β-strand and the first α-helix would proceed in a counterclockwise direction (shown with the white arrow)

The topology of the Top7 protein has never been observed in natural proteins. The overall structure was designed de novo by Kuhlman et al., who intentionally selected a novel topology for the protein. Once the topological constraints were fixed, Kuhlman et al. used a “computational strategy that iterates between sequence design and structure prediction” to design, in silico, a 93 amino acid protein (Top7) with a particular predicted three-dimensional structure. Kuhlman et al. found that the protein could be expressed as a highly soluble monomeric protein with a 3-D structure that agreed with the predicted in silico structure. Indeed, the experimentally-determined structure of the protein backbone has a root mean square deviation (“RMSD”) of only 1.1 Å from the in silico structure. Top7 is also exceptionally stable, as heating the protein to 98° C. does not appear to denature the protein. Even in the presence of 4.8 M of the denaturant guanidine hydrochloride, temperatures exceeding 80° C. are required to fully denature the protein.

Intriguingly, it has also been reported that the C-terminal 49 amino acids of Top7 can also be efficiently expressed as an exceptionally stable homodimer (Dantas et al. (2006) J. Mol. Biol. 362:1004-1024). These 49 amino acids include the third β-strand, the second α-helix, and the last two β-strands of Top7 (i.e. structural elements 4, 5, 6, and 7, in the order βαββ). Each subunit retains the same fold that the corresponding sequence has in full-length Top7, with one α-helix packed against three strands of a β-sheet. Like Top7, the homodimer forms a globular two layer structure with two α-helices in one layer packed against a second layer of antiparallel β-strands, although whereas the β-sheet of Top7 has five antiparallel β-strands, the homodimer has six. Like Top7, the homodimer is extremely stable, as Dantas et al. reported that the secondary structure for a 12 μM solution of the C-terminal fragment (“CFr”) appears unchanged at 98° C. or in 3M guanidine hydrocholoride and that, even in 4 M guanidine hydrochloride, temperatures exceeding 80° C. are required to fully denature the protein. Dantas et al. succeeded in further stabilizing CFr by introducing a disulfide bond connecting the N- and C-termini of the fragment; this stabilized fragment, termed “SS.CFR,” only begins to unfold at 6.5M guanidine hydrochloride, a concentration of denaturant that almost completely unfolds CFr and Top7.

As the Top7 structure was designed de novo, it is perhaps unsurprising that widely differing amino acid sequences can be selected in silico to achieve the Top7 fold. For example, Dallüge et al. used a different algorithm, based on tetrapeptide backbone formations, to create de novo polypeptide sequences predicted to adopt the Top7 fold ((2007) Proteins 68:839-849). Two of their designed polypeptide sequences, M5 and M7, each fold into proteins that were reported to be stable at all accessible temperatures in the absence of denaturant and that were not fully denatured at 80° C. in the presence of 4M guanidine hydrocholoride (or even 6M guanidine hydrochloride, for M7). Neither protein is more than 30% identical to the amino acid sequence of Top7.

### Insertions/Heterologous Sequences

Thus, existing technologies permit the design of proteins of widely varying sequence, each nevertheless demonstrating proper folding and a stability permitting significant latitude in the introduction of heterologous sequences. These heterologous sequences can be used to replace amino acids in the secondary structure elements of the scaffolds, or in the interconnecting loops. Alternatively, or in addition, heterologous sequences can be inserted into the scaffold molecule, preferably within one or more of the interconnecting loops. Heterologous sequences can also be appended to the N- and/or C-terminus of the scaffold.

As shown in FIG. 3, full-length Top7 includes six interconnecting loops, which FIG. 3 identifies as loops 12, 23, 34, 45, 56, and 67. For scaffolds having a complete Top7 structure, heterologous sequences can be inserted into any one of these loops, or into any combination of these loops. Proteins that include only a portion of the Top7 structure, such as CFr or derivatives thereof (e.g. SS.CFr) can also be used as scaffolds. When only a portion of the Top7 structure is present, heterologous sequences can be inserted into any one of the loops present in that portion. Thus, for example, CFr includes loops 45, 56, and 67, any or all of which could incorporate heterologous sequences.

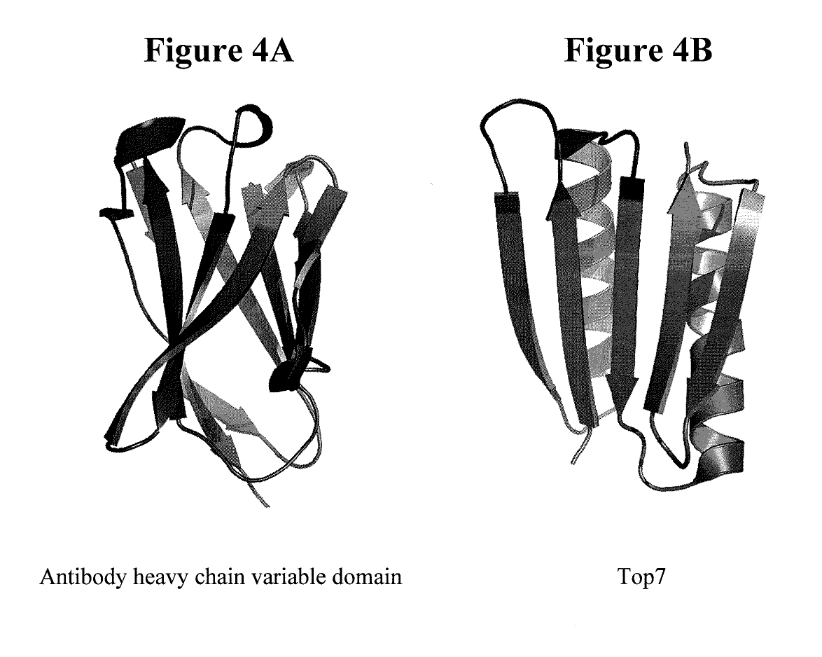

In some embodiments of the invention, heterologous sequences are introduced into multiple loops of the scaffold, preferably on the same end of the protein. Three loops are present at each end of the protein, reminiscent of the CDRs on antibody variable domains. As shown in FIG. 4, the loops of Top7 and the loops of antibody CDRs are are more or less similarly oriented. In fact, loop 12 in Top7 is almost exactly the same as CDR3 in a V_{H} domain. Thus, scaffolds incorporating one or more features of Top7 can be used like the framework of an antibody variable domain to present loops of varying sequence, some of which will separately or in combination have a useful affinity for a target molecule.

### Amino Acid Sequences

Because amino acid sequences with little sequence identity (e.g. less than 30% identity, as observed in M5 and M7) can nevertheless fold into stable structures suitable for use as scaffolds, a correspondingly wide variety of amino acid sequences are embraced by the present invention. Beyond Top7, useful scaffolds include, for example, CFr; SS.CFr; proteins disclosed in Dallüge et al., including but not limited to M5 and M7. The scaffold can incorporate any mutation that does not preclude proper folding. For example, of the seventeen point mutations engineered into Top7 in Watters et al. (2007) Cell 128: 613-624, none of them (K41E/K42E/K57E; F17Q/Y19L; G14A; Y21L; L29A; N34G; V48A; F63A; A64G/A65G; L67A; G85A; and V90A) precluded proper folding of the protein. Unsurprisingly, as Top7, M7, and other, related proteins are exceptionally stable, they can incorporate several mutations without losing their only required feature, i.e., their ability to fold into a stable structure.

One scaffold related to Top7 is referred to herein as “RD1.3/1.4 Consensus” and is presented as SEQ ID NO:5. RD1.3/1.4 Consensus represents a variant of Top7 engineered to incorporate several amino acid substitutions. Another scaffold related to Top7 is referred to herein as RD1-DI-DeLys, and represents a variant of RD 1.3 engineered to reduce the number of lysine residues present in the protein, thereby facilitating site-specific modification of lysine residues and reducing opportunities for proteolysis. RD1-DI-DeLys has also been engineered to reduce the availability of potentially immunogenic epitopes. The amino acid sequence of RD1-DI-DeLys is presented as SEQ ID NO:2. Accordingly, some scaffolds that can be used in the practice of the invention have amino acid sequences resembling portions of RD1.3 and/or RD1-DI-DeLys. Certain portions of RD1.3/1.4 Consensus from its seven structural elements have been concatenated and presented in SEQ ID NO:6; corresponding portions of RD1-DI-DeLys have been concatentated and presented in SEQ ID NO:3. For each of SEQ ID NO:3 and SEQ ID NO:6, amino acids 1-5 correspond to a portion of the first β-strand; amino acids 6-8 correspond to a portion of the second β-strand; amino acids 9-20 correspond to a portion of the first α-helix; amino acids 21-23 correspond to a portion of the third β-strand; amino acids 24-32 correspond to a portion of the second α-helix; amino acids 33-37 correspond to a portion of the fourth β-strand; and amino acids 38-42 correspond to a portion of the fifth β-strand.

As it is understood that the ends of the protein, including the ends of the structural elements and the interconnecting loops, can be varied significantly or replaced completely in a scaffold, these portions of RD1.3/1.4 Consensus and RD1-DI-DeLys have been omitted from SEQ ID NO:6 and SEQ ID NO:3. It is nevertheless understood that the structural elements will be connected by interconnecting loops which will be, in most instances, amino acid sequences including at least as many amino acids as are normally found separating those structural elements (e.g. the number of amino acids separating the corresponding portions of Top7. Thus, for example, a scaffold of including an amino acid sequence formula B(1)-L(12)-B(2)-L(23)-A(3)-L(34)-B(4)-L(45)-A(5)-L(56)-B(6)-L(67)-B(7) can be used, where B(1), B(2), A(3), B(4), A(5), B(6), and B(7) correspond generally to amino acids 1-5, 6-8, 9-20, 21-23, 24-32, 33-37, and 38-42 of SEQ ID NO:3, or a sequence at least 80% identical to SEQ ID NO:3, or SEQ ID NO:6, or a sequence at least 90% identical to SEQ ID NO:6. The minimum lengths of L(12), L(23), L(34), L(45), L(56), L(67) are generally 10, 7, 9, 10, 7, and 4 amino acids, respectively. Often, one, two, three, or more of L(12), L(23), L(34), L(45), L(56), and L(67) exceed their minimum lengths, such as by 1-3 amino acids, by 2-6 amino acids, by 3-8 amino acids, by 4-12 amino acids, or by 5-14 amino acids or more.

Alternatively, as demonstrated with CFr, any portion of a Top7-like molecule that is able to fold reliably can be used. Thus, for example, a scaffold including an amino acid sequence of the formula B(4)-L(45)-A(5)-L(56)-B(6)-L(67)-B(7) can be used, where B(4), A(5), B(6), and B(7) correspond generally to amino acids 21-23, 24-32, 33-37, and 38-42 of SEQ ID NO:3, or a sequence at least 80% identical to amino acids 21-42 of SEQ ID NO:3, or at least 90% identical to amino acids 21-42 of SEQ ID NO:6.

### Libraries and Selection

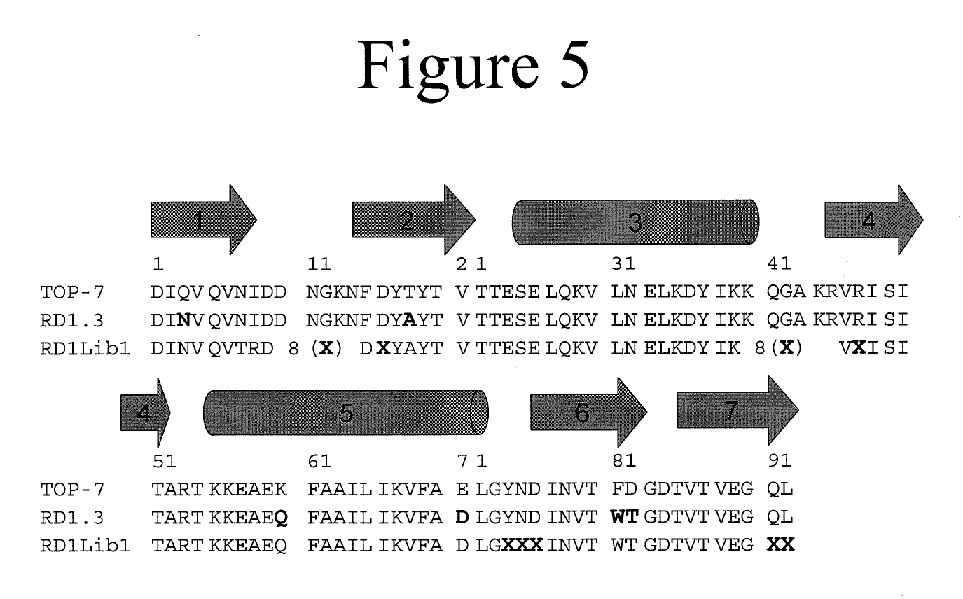

One advantage of a stable scaffold molecule is that it permits the preparation of libraries of proteins presenting randomized sequences. Individual proteins with a desired property, such as the ability to bind to a preselected target molecule, can then be isolated from the library. The randomized sequences can include randomized loop sequences, including randomized insertions into loop sequences, and can also include randomized sequences in the structural elements of the scaffold protein, in any combination. One example of a protein library, denoted “RD1Lib1,” is depicted in FIG. 5 and its sequence is presented as SEQ ID NO:7. As shown in FIG. 5, RD1Lib1 replaces five amino acids from loop 12 with eight random (X) amino acids; randomizes one amino acid position in structural element 2, replaces six amino acids from loop 34 with eight random amino acids, randomizes one amino acid in structural element 4, randomizes three amino acids in loop 56, and randomizes the last two amino acids of the protein. Beyond randomizing any combination of loops 12, 23, 34, 45, 56, and 67, a protein library can include randomization or other modification at positions corresponding, for example, to any one of the following positions of Top7: N7, D16, R47, N78, and/or E89 of the β strands on the North End; N3, T20, S49, T80, and T87 of the β strands on the South End; K39 through Q41 and A70 and D71 of the α-helices of the North End; and/or E26 and K55- E57 of the α-helices of the South End. In addition, residues that are internal and near the ends could be randomized, in order to provide a differently-shaped ‘foundation’ for the binding surface. For example, amino acids at positions corresponding to one or more of 18, V46, 177, F69, and I38 of Top7 could be randomized in a protein library.

The N- and C- termini of a protein library can also be randomized with respect to composition and length. For example, the N- or C-terminus of the protein could be shortened by one residue, compared to RD1.3, or extended by up to ten residues. Randomized location of stop codons at the end of the protein could be used to generate this length diversity at the C-terminus.

In some cases, the randomness of an amino acid position can be restricted, e.g. to avoid cysteine residues, to avoid lysine residues, or to favor hydrophilic amino acids to reduce immunogenicity.

A protein library can be constructed in the context of plasmid vectors or phage vectors, for example. It is particularly useful to construct such vectors and host systems in a way that members of the protein library that bind to a given target can be selected. For example, display systems using single-stranded phage such as M13 or fd, double-stranded phage such as T7 or lambda, flagella or other surface proteins of bacteria such as E. coli, ribosome-based display, messenger RNA display, surface proteins such as Aga2 of yeast, or protein-only systems can be used.

Once a protein library has been prepared, members of the library can be selected based on affinity for a preselected target molecule, such as a nucleic acid, an antibody variable domain, a sugar, an oligosaccharide, a lipid, or another organic or inorganic compound. In one type of selection protocol, a protein library is expressed on a phage such as M13 or T7 according to standard techniques. It should be noted than an advantage to Top7-related scaffolds is that both the N-terminus and C-terminus are available for genetic fusion to a host protein, and the opposing end may be used for loop insertion and peptide fusion. Protein libraries described in the Examples have their N-terminus fused to T7 coat protein and C-terminus and adjacent binding end available. The reverse orientation is also practicable, so that the binding end would be oriented on the N-terminal end of the scaffold, and its C-terminus fused to a display protein, such as the gene III protein of M13 bacteriophage.

As one example, a phage expression scaffold library can be applied to an immobilized target under conditions that favor binding, one or more washing steps are executed, and then bound phage are eluted using conditions such as high salt, low or high pH, a detergent such as SDS, or another solvent conditions dictated by particular needs of the experiment. The eluted phage are expanded by, for example, growth in a bacterial host. PCR-based techniques can also be used to expand nucleic acids encoding potential binding proteins after a binding/selection step, followed by recloning into the appropriate vector and packaging into a phage particle or transformation into a bacterial host. After amplification, the population that has been enriched for those phage encoding specific binding proteins is again exposed to the preselected target molecule, binders selected in this new round, and the cycle of recovery is repeated. This cycle is optionally repeated, for example, three to five times. If desired, the success of the enrichment steps can be monitored by titering the number of phage that are retained after each step; the titer should increase if enrichment is occuring. At a certain point, which may be indicated by titering the number of phage that adhere after each binding step or which may be determined by routine experimentation, it is useful to test individual candidates for their ability to bind to a given target. Examples 5 and 6 describe particular methods for such analysis, although a wide variety of methods may be used.

In some circumstances, it is useful to select binding proteins from a library and then recombine randomized portions of members of the selected population with each other to generate binding proteins that may have higher affinity.

### Nucleic Acids

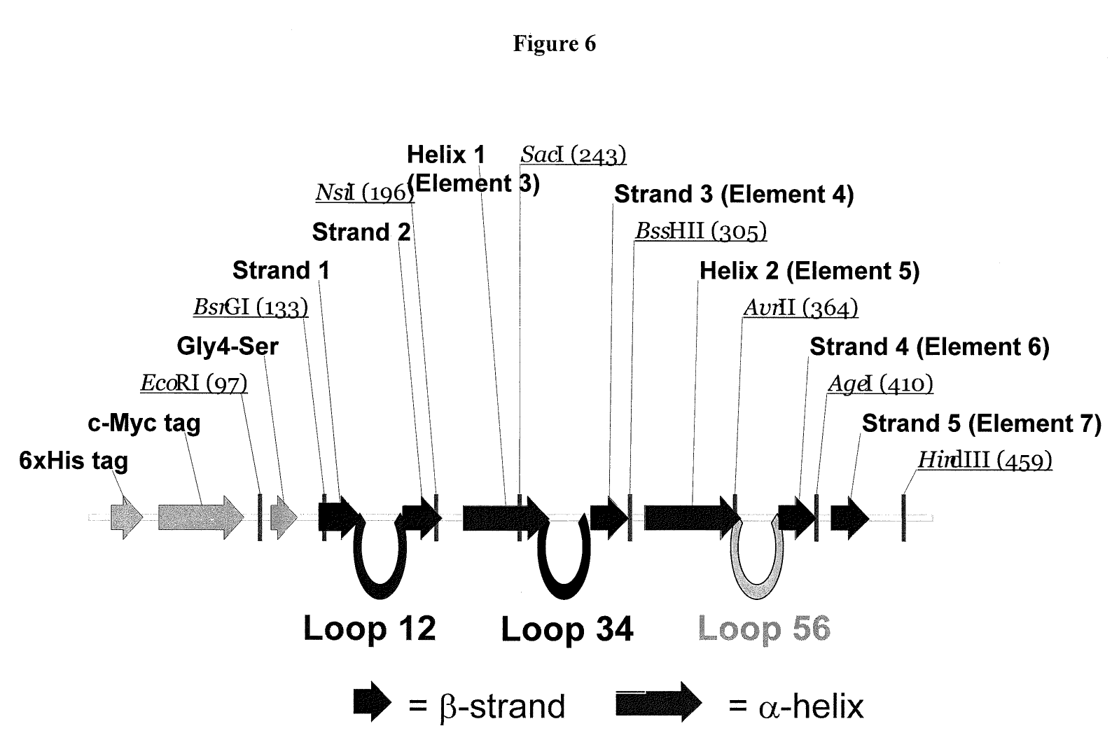

Proteins of the invention can be expressed using any suitable nucleic acid encoding the protein or protein library, in any suitable prokaryotic (bacterial) or eukaryotic (e.g. yeast, insect or mammalian, such as human, primate, hamster, etc.) system. For protein libraries, it can be advantageous to incorporate restriction sites to facilitate excision and transfer of the nucleic acid encoding the protein. Appropriately placed restriction sites can also facilitate the selective excision of one or more loops or other randomized sequences. One exemplary nucleic acid is depicted in FIG. 6. FIG. 6 depicts a nucleic acid with insertion sites in loops 12, 34, and 56. Each loop is flanked by two restriction sites, permitting the selective excision (and/or insertion) of any loop sequence of interest.

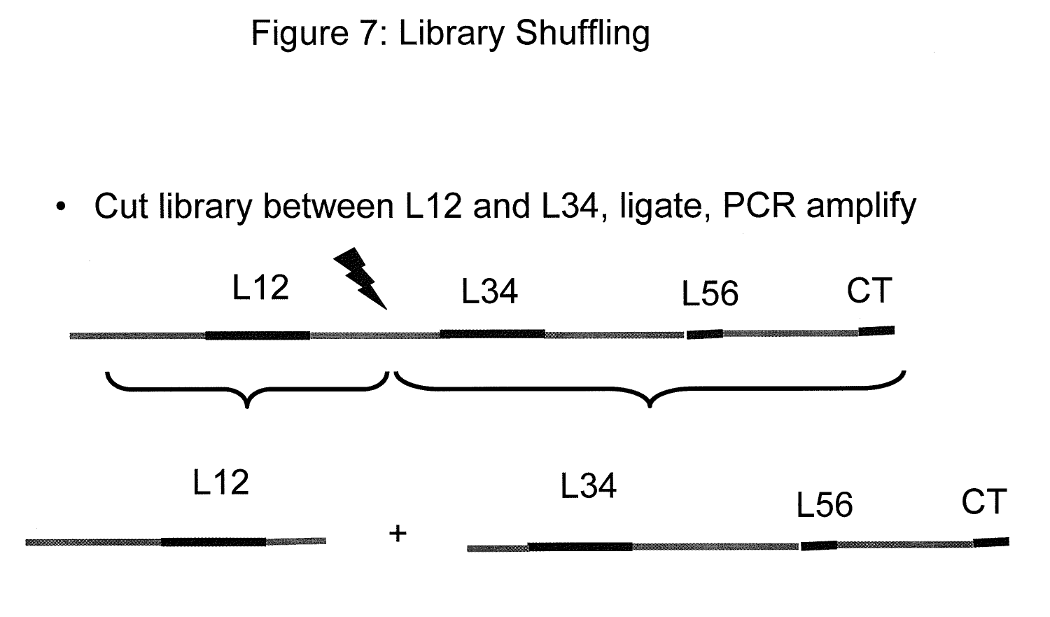

For example, intervening restriction sites can be used for “shuffling” loops among members of a library. One example is depicted in FIG. 7. As shown in FIG. 7, after members of a library have been selected for proteins with a particular property (such as an affinity for a particular target), library members can be cleaved at one or more internal restriction sites and religated, leading to the recombination and reshuffling of loops among library members, which may lead to the identification of higher-affinity interactors.

Throughout the description, where compositions are described as having, including, or comprising specific components, it is contemplated that compositions also consist essentially of, or consist of, the recited components. Similarly, where processes are described as having, including, or comprising specific process steps, the processes also consist essentially of, or consist of, the recited processing steps. Except where indicated otherwise, the order of steps or order for performing certain actions are immaterial so long as the invention remains operable. Moreover, unless otherwise noted, two or more steps or actions may be conducted simultaneously.

### EXAMPLES

The invention is explained in more detail with reference to the following Examples, which are to be considered as illustrative and not to be construed so as to limit the scope of the invention as set forth in the appended claims.

### Example 1

### Thermodynamic Properties of Scaffolds with Peptide Insertions

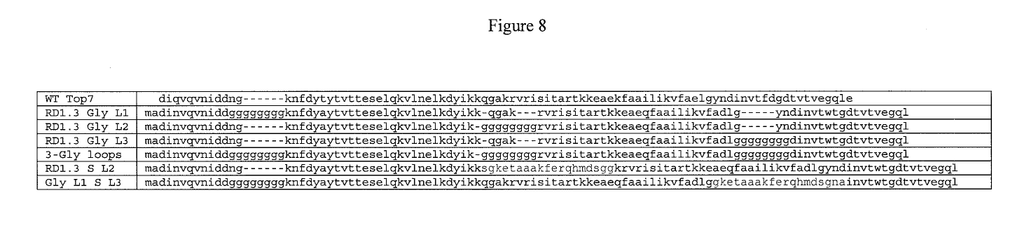

To confirm the suitability of RD1.3 as a scaffold containing large, random peptide loops, loops 12, 34, and 56 were replaced with eight glycines each. These were chosen because glycine is the most disruptive of all amino acids from a backbone entropy standpoint—if the protein still folds and is stable with 8 glycines, it should fold with most other reasonably soluble random sequences. Another sequence, the 15 amino acid loop “S-peptide”, was also inserted into the RD1.3 protein, alone and in combination with glycine loops. S-peptide is part of the RNase-S enzyme that is known to bind to the truncated enzyme and complete it, thereby restoring function. This peptide as a loop insertion would provide both a binding and an enzymatic assay to demonstrate the ability of RD1.3 to display useful loops. The amino acid sequence of each of these test proteins is shown aligned to the Top7 sequence in FIG. 8.

Each protein tested, with the glycine loops or the glycine loops and the S-peptide loop, was soluble and homogenous. There was little aggregation, even after multiple freeze-thaw cycles and long term storage at 4° C. Each protein solution was stable at 4-5 mg/mL. Thus, even even large, high entropy insertions are well-tolerated, presumably because of the substantial stability of the starting structure of RD1.3.

### Example 2

### Designed Scaffolds

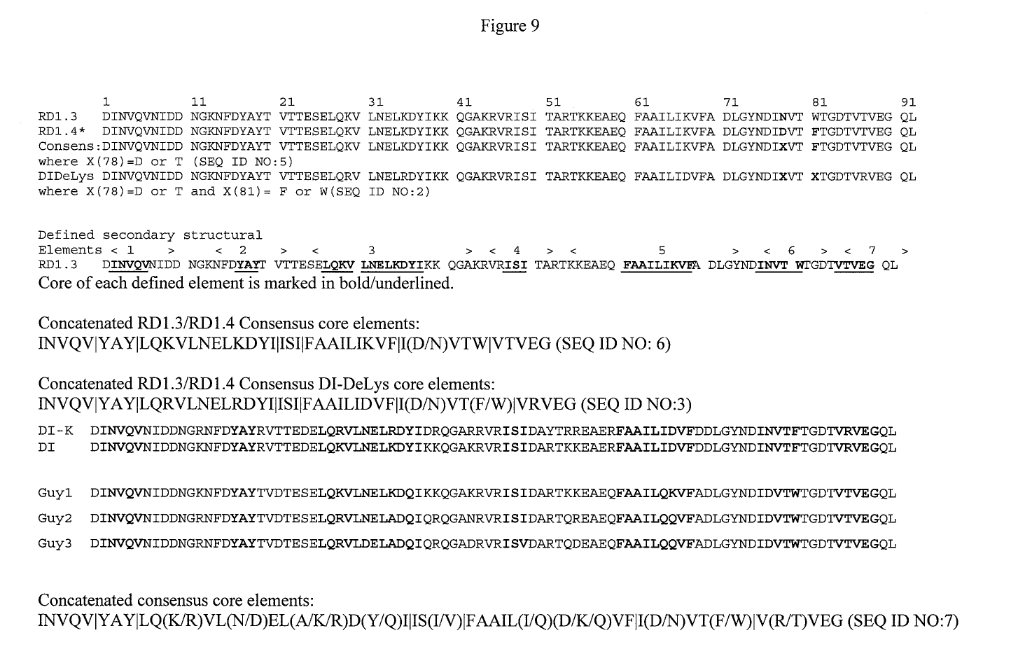

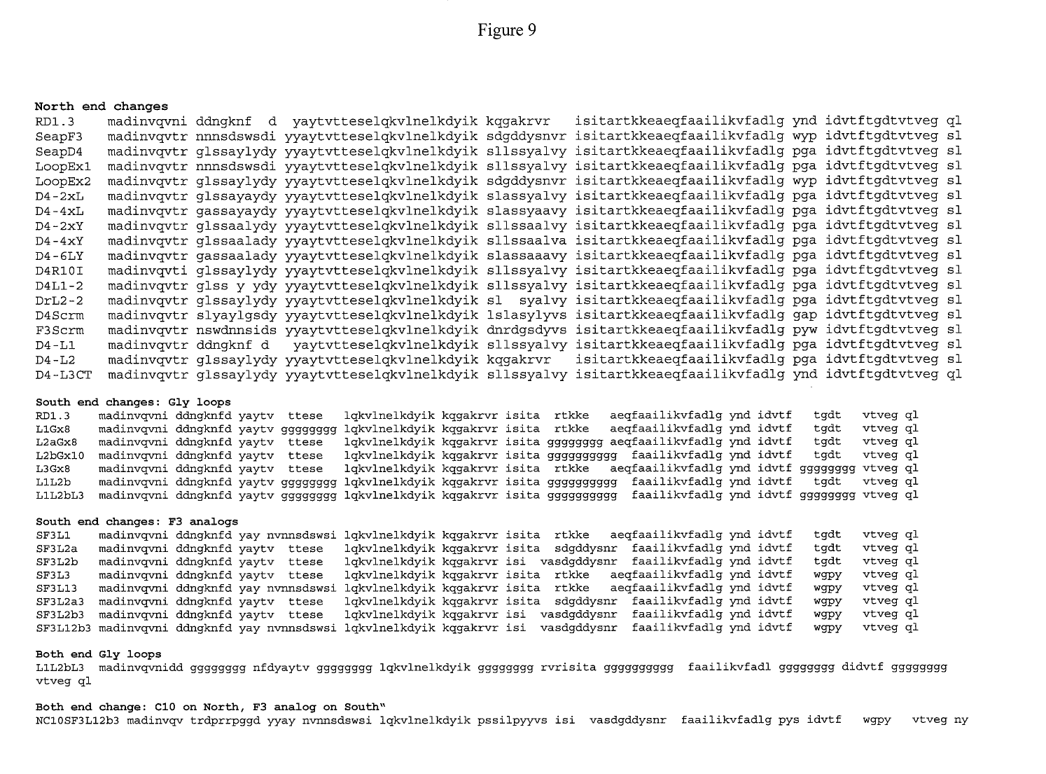

A variety of proteins related to Top7 were designed for use as protein scaffolds. The amino acid sequences of the proteins are depicted in the alignment shown in FIG. 9. As is evident in FIG. 9, insertions in each loops 12, 23, 34, 45, 56, and 67 were successfully designed, with or without point mutations at various positions throughout the scaffold. It is contemplated that these proteins, and other related proteins at least 50% identical, at least 60% identical, at least 70% identical, at least 80% identical, at least 90% identical, or at least 95% identical to one or more of these proteins or to the α-helices and β-strands of one or more of these proteins, are useful as scaffolds and as the basis for protein libraries incorporating one or more heterologous sequences as described in this application.

### Example 3

### Design and Synthesis of Exemplary Library RD1Lib1

To construct a library of genes with variable peptide loops, the following techniques were employed. First, a set of amino acids and frequency distributions were chosen, as indicated in the Table below.

| Amino Acid | Percentage |
|---|---|
| Tyr | 25 |
| Ser | 17 |
| Leu | 10 |
| Ala | 10 |
| Asn | 10 |
| Gly | 5 |
| Ile | 5 |
| Asp | 5 |
| Arg | 5 |
| Pro | 5 |
| Trp | 3 |

In this particular library construction, only 11 amino acids were chosen. It will be apparent to those skilled in the art of protein engineering that a variety of amino acids and distributions can be used. It is often useful to avoid the use of cysteine, because this amino acid may lead to the formation of undesired disulfide bonds, and selenocysteine, because this amino acid is encoded by a UGA codon that may also be interpreted as a stop codon.

The oligonucleotides listed below were obtained from a commercial supplier (TriLink BioTechnologies (San Diego, Calif.)).

SEQ. E1: L1

(SEQ ID NO: 1)

GCT CCT GAT GTA CAG GTA ACC CGT (XXX)₈ GAC XXX TAC
TAT GCA TAC ACG GTG ACC
SEQ. E2: L2

(SEQ ID NO: 4)

CTG AAC GAG CTC AAA GAC TAC ATT AAA (XXX)₈ GTT XXX
ATT TCT ATT ACC GCG CGC ACT AAA
SEQ. E3: L3

(SEQ ID NO: 8)

AA GTA TTC GCT GAC CTA GGA (XXX)₃ ATT AAC GTC ACT
TGG ACC GGT GAC ACA
SEQ. E4: CTERM (”CT”)

(SEQ ID NO: 9)

ACT TGG ACC GGT GAC ACA GTA ACA GTA GAA GGA (XXX)₂
TAA TAA CTC GAG GAA GCT TGG

Codons marked “XXX” are insertions from the codon mix described above. Restriction sites are underlined. For each of the four oligonucleotides with random segments, a pair of PCR primers was synthesized (shown below) that bind to the fixed tails. Restriction enzyme recognition sites are underlined, and the appropriate restriction enzymes are listed below the sequence.

L1:

(SEQ ID NO: 10)

| | |
|---|---|
| 5′ GCT CCT GAT GTA CAG GTA ACC CGT 3′ | (L1-F) |
| BsrGI | |

(SEQ ID NO: 11)

| | |
|---|---|
| 5′ GGT CAC CGT GTA TGC ATA GTA 3′ | (L1-R) |
| NsiI | |
| L2: | |

(SEQ ID NO: 12)

| | |
|---|---|
| 5′ CTG AAC GAG CTC AAA GAC TAC ATT AAA 3′ | (L2-F) |
| SacI | |

(SEQ ID NO: 13)

| | |
|---|---|
| 5′ TTT AGT GCG CGC GGT AAT AGA AAT 3′ | (L2-R) |
| BssHI | |
| L3: | |

| | |
|---|---|
| (SEQ ID NO: 14 | ) |

| | |
|---|---|
| 5′ AA GTA TTC GCT GAC CTA GGA 3′ | (L3-F) |
| AvrII | |

(SEQ ID NO: 15)

| | |
|---|---|
| 5′ TGT GTC ACC GGT CCA AGT GAC GTT AAT 3′ | (L3-R) |
| C-term(CT): | |

(SEQ ID NO: 16)

| | |
|---|---|
| 5′ ACT TGG ACC GGT GAC ACA GTA ACA GTA | (CT-F) |
| GAA GGA 3′ | |

(SEQ ID NO: 17)

| | |
|---|---|
| 5′ CCA AGC TTC CTC GAG TTA TTA 3′ | (CT-R) |
| XhoI | |

In this and all subsequent examples, PCR amplification was performed under standard conditions, and the reactions monitored by agarose gel. When the product band was clearly visible and did not significantly increase in intensity between two samples taken two cycles apart, the reaction was considered complete. In order to ensure clean double-stranded DNA during these amplification steps, the following modification to standard PCR procedures was generally used. When DNA is amplified by PCR, during later cycles the re-annealing of full length DNA may compete with primer annealing. In the case of a diverse oligonucleotide pool, this effect can lead to unpaired DNA regions, where the fixed portions anneal, leaving the unmatched random regions as bulges. Particularly if the bulge is near a restriction site that is to be used for cloning, such single-stranded regions may reduce the efficiency of ligation. To create completely double-stranded DNA without the unpaired regions, fully amplified PCR product was diluted three-fold into fresh PCR mix with the same primers, and a single cycle of denaturation, primer annealing, and elongation was performed. All restriction digestion was performed with enzymes purchased from New England Biolabs (Beverly, Mass.), using supplied buffers and occasionally modified as described.

The individual loops with random segments were combined into a pool of genes encoding essentially full-length proteins as follows. Each of the four oligonucleotide pools was amplified using the appropriate forward and reverse oligonucleotides listed above. From the L3 and CT PCR reactions, one pL of each reaction was then combined in a fresh 100 μL PCR reaction, and further amplified using oligonucleotides L3-F and CT-R. This longer oligonucleotide pool, comrising both the L3 and CT diversity elements, was called L3/CT.

The L3/CT reaction was cleaned up with Phenol/Chloroform/Isoamyl alcohol (25:24:1) extraction, followed by 2× chloroform extraction and ethanol precipitation. The DNA was dissolved in buffer then cleaved with restriction enzymes AvrII and XhoI in a single reaction in NEB buffer 2 supplemented with BSA, at 37° C., following the instructions of the manufacturer. The L1 and L2 reactions were likewise cleaned up with Phenol/Chloroform/Isoamyl alcohol (25:24: 1) extraction, followed by 2× chloroform extraction and ethanol precipitation. L1 DNA was digested with BsrGI in NEB buffer 2 plus BSA at 37° C., then 1/20 volume of 1M NaCl and 1/25 volume of 1M TRIS-HCl (pH 7.9) added, and the DNA further digested with NsiI at 37° C. L2 DNA was digested with SacI in NEB buffer 1 plus BSA at 37° C., then BssHII was added and the sample digested at 50° C., according to the instructions of the maunfacturer. Three aliquots of pUC19 containing the scaffold gene were made. The first aliquot was digested with restriction enzymes AvrII and XhoI in a single reaction in NEB buffer 2 supplemented with BSA, at 37° C., following the instructions of the manufacturer. The second aliquot was digested with BsrGI in NEB buffer 2 plus BSA at 37° C., then 1/20 volume of 1M NaCl and 1/25 volume of 1M TRIS-HCl (pH 7.9) added, and the DNA further digested with NsiI at 37° C. The third aliquot was digested with SacI in NEB buffer 1 plus BSA at 37° C., then BssHII was added and the sample digested at 50° C., according to the instructions of the maunfacturer. No alkaline phosphatase was added to any of the above reactions.

L1, L2, and L3/CT digested DNA were separately gel purified using 3% low-melting agarose gels made with Gel-Star dye (Cambrex, Walkersville, Md.), following the instructions of the manufacturer. Correct bands were excized and the DNA extracted using warm phenol followed by choloroform (2×) and ethanol precipitation. Each double-digested pUC19/RD1 aliquot was separately gel purified in 0.8% agarose gels made with Gel-Star dye, following the instructions of the manufacturer. Bands were excised and the DNA extracted using a Qiagen gel extraction kit.

The next step in the construction of the library was to ligate each of the three trimmed DNAs with diversity segments into the purified linearized vector that had been digested with the same two restriction enzymes as the DNA to be inserted. For each of the three ligations to be performed, a 20 μL ligation reaction was set up with 50 nanograms of linearized vector, a three-fold molar excess of insert DNA containing diversity, and the appropriate buffer and enzyme (New England Biolabs, Beverly, Mass.), according to the instructions of the manufacturer. The result of this ligation was a set of three circularized vector DNA pools, each containing the RD1 gene with diversity in one of the three regions (L1, L2, or L3/CT). Since no alkaline phosphatase was used at any point, the circularized vector should in general have no nicks, but would not be tightly supercoiled.

Bacterial transformation is an inefficient process, wherein the majority of the circularized vector is not successfully transformed. In order to preserve the maximum library complexity, the following procedure was used to extract and amplify virtually all of the successfully ligated DNA diversity. 5 μL of the ligated material was put directly into a 100 μL PCR reaction with primers that annealed to the pUC vector on either side of the insert (M13For and M13Rev). PCR was performed, with 5 μL timepoints removed every two cycles after about 10 cycles. Based on the amount of DNA present in the timepoints, the minimum amount of PCR-competent ligated library DNA present in the mix before the initiation of PCR was back-calculated, based on the maximum rate of amplification of doubling each cycle. The calculation used the following equation: C>=m /(2̂n), where C is initial complexity (number of molecules from which genes containing diversity can be extracted by PCR), and m is the number of molecules in the PCR reaction after n cycles of PCR. As an example, the fragment from pUC19 containing scaffold amplified by PCR with M13For and M13Rev is approximately 590 base pairs. After n cycles of PCR the total amount of DNA of length 590 in the PCR reaction can be measured by comparing the intensity of the band (from the ‘n’ timepoint) with the bands from a quantitative marker such as Low Mass (Invitrogen, Carlsbad, Calif.). If after for example 10 cycles (n=10) the band has 50 ng of DNA, from 4 μL of PCR (12.5 ng/μL), then the remaining e.g. 80 μL of PCR reaction has 80 μL * 12.5 ng/μL=1000 ng=1 μg. A 590 basepair double-stranded DNA fragment has a molecular weight of approximately 590 b.p. * (660 AMU/b.p.)=3.9E+05 AMU/molecule. To calculate the number of molecules in 1 gram: (6.02E+23 molecules/mole)/(3.9E+05 grams/mole)=1.5E+18 molecules/g=1.5E+12 molecules/μg. To calculate the minimum initial complexity C, m=1.5E+12 and n=10. Thus, C=1.5E+12/(2̂10)=1.5E+09. For L1 and L2, if C exceeded 1.0E+09, the complexity was considered sufficient and the ligated DNA was used for the next step. For L3/CT, C>10E+06 was deemed sufficient.

Assembly of the full RD1Lib1 library: Primers were designed to asymmetrically amplify the scaffold gene from pUC19 vector. pUC-Top+600 is approximately 600 b.p. removed from the insert (on the side containing the N-terminus of the expressed protein), while pUC Bottom+150 is approximately 150 b.p to the other side of the insert. When a scaffold gene is amplified using these primers, the PCR fragment can be cut by any enzyme with a unique recognition site within or bordering the gene, and the two resulting fragments will differ by at least 100 bp, so they can be readily separated by agarose gel electrophoresis.

The final mixture of L1.1/L2.1/L3.1/CT reaction products was estimated to have a complexity of at least 5×10⁹.

T7 Select Phage Display System Packaging Kits(P/N 70014) and 10-3 T7Select vector DNA (P/N 70548) were obtained from Novagen (San Diego, Calif.) and a library using the L1.1/L2.1/L3.1/CT reaction product was constructed according to the instructions of the manufacturer.

The L1.1/2.1/L3.1/CT reaction product was digested with EcoRI and HindIII, gel purified, then ligated into 10-3b T7 vector arms at a molar ratio of 3:1 insert:phage DNA. After overnight ligation of 20 ug of vector arms in a 200 microliter volume, the ligation reaction was then mixed with a total of 1 ml of packaging extracts and incubated for 2 hours at room temperature, diluted 9:1 with sterile LB, then titered, all according to the manufacturer's directions. The titer gave a total number of packaged phage of 1.5×10⁹. Subsequent sequencing of the library revealed that about 30% of the genes had a frame shift, so the library complexity of full length scaffold genes was about 1×10⁹. The phage were expanded in 1 liter of E. coli, strain 5403 (CalBiochem), and upon lysis the phage were concentrated twice by PEG precipitation followed by CsCl gradient purification and dialysis, as described by the phage library kit instructions.

It should be noted that several variations on this procedure for creating a library can be performed. For example, for the library described above, the 10-3b version of phage T7 was used; this version expresses about 5 to 15 copies of fusion protein on the surface of each phage particle, according to the manufacturer (Novagen). It is also possible to use other phage genomes such as 1-1b, which display 0.1 to 1 fusion protein/phage particle, according to the same manufacturer.

### Example 4

### Selection of Binding Proteins

Individual proteins that bind to specific targets were isolated from the T7-based RD1Lib1 library constructed in Example 3 by the following procedures. In outline, the general procedure was to bind a target protein to beads, mix the T7-RD1Lib1 library with the beads, wash, elute the bound T7 phages, infect E. coli with the eluted phages to expand this population, and proceed through several more cycles of binding, elution, and expansion until a significant fraction of the phage population expressed a protein that binds to the target. At this point, individual library members were tested for their ability to bind to the target and optionally to not bind to related target molecules. In some cases, negative selection steps were included. For example, when isolating proteins that bind specifically to a particular antibody V region pair, a negative selection step against an antibody with the same constant regions but different V domains was generally first performed before selecting for proteins that bind an antibody with the desired V region target.

For example, proteins were identified that bind specifically to the V regions of an anti-CD19 antibody (see U.S. Patent Application Publication No. US2007/0154473); a humanized 14.18 antibody (see U.S. Pat. No. 7,169,904); or an anti-EpCAM antibody (see U.S. Pat. No. 6,969,517). The antibody proteins were produced from genetically engineered mammalian cell lines as described.

The following specific procedures were used for specific selections in the isolation of proteins that bound to the anti-CD 19 antibody. The overall strategy was to perform a round of positive selection under low-stringency conditions, amplify the selected phage, perform a round of negative selection followed immediately by a second round of positive selection under more stringent conditions, another round of amplification, a reassortment step in which the DNAs encoding the N- and C-terminal portions of the selected RD1 populations are recombined and subsequently placed in a low-copy T7 expression vector, followed by a round of positive selection and two rounds of negative plus positive selections, with amplifications after rounds of positive selection. At the end of this process, individual library members were tested as described in Examples 5 and 6.

To produce a binding substrate, the anti-CD 19 antibody was first bound to streptavidin-coated DYNAL beads (product 112.06 from Invitrogen Corp., Carlsbad, Calif.) using a biotinylated goat anti-human antiserum as a bridge (Jackson Immunolabs, Md.). To prepare for a single round of selection, about 100 μL of beads at at 6.7×10⁸ beads/ml were placed in a 1.5 ml plastic tube in a magnetic rack and allowed to settle for about 1 minute until all of the beads were tightly held against the side of the tube. The supernatant was removed, 1 ml of TBS (Pierce) was added, the beads were mixed into the TBS, the beads again allowed to settle in the magnetic rack, supernatant withdrawn, 1 ml of TBS again added, and the beads again allowed to settle. Finally the beads were resuspended in about 30 μL of TBS. About 10 μg of biotinylated goat anti-human antibody in the form of 20 μL of a glycerol stock were added to the beads. The slurry was placed on a rotator and allowed to rotate for about 6 to 9 hours at room temperature. The beads were then washed 4 times in 1 ml of TBS and resuspended in 30 μL of TBS.

To initially select library members that bound to the V regions of an anti-CD 19 antibody, about 10 μg of the anti-CD19 antibody was mixed with the beads. The tube was placed on the rotator overnight at 4° C. to allow the anti-CD 19 antibody to bind to the goat anti-human IgG on the beads. The following morning, the beads were washed twice in 1 ml TBS as described above, resuspended in 3% BSA in PBS, rotated for another 2 hours at room temperature, washed twice in 1 ml of TBS, and resuspended in a solution containing T7 phage particles prepared by mixing and incubating 100 μL of a T7-RD1Lib1 library with a titer of 5×10¹¹ to 10¹² plaque-forming units per ml and 11 μL of 30% BSA for 2 hours at room temperature. The mixture containing the phage and the beads was incubated for about 30 to 60 minutes at room temperature on the rotator. The beads with adsorbed phage were then washed six times in 1 ml TBS with 0.05% Tween 20 at room temperature. After each addition of the TBS-Tween, the beads were left suspended for 1 minute, then magnetically separated as described above, the supernatant withdrawn, and fresh TBS-Tween added. After every other wash, the mixture was moved to a new tube. After the final wash, the bound phage were eluted from the beads by the adding 100 μL of 1% SDS in TBS, incubating for 5 minutes, and removing the supernatant from the beads magnetically as described above. The 100 μL of supernatant were immediately added to 900 μL of TBS.

The selected phages were amplified as follows. About 20 to 30 μL of the eluted phage were withdrawn for titering, and the remainder was added to 35 mls of E. coli 5403 exponentially growing at 37° C. in rich medium supplimented with 50 mg/l ampicillin at an O.D. of about 0.5. The culture was aerated at 37° C. until lysis, which usually occurred after about 2-4 hours and was defined by a drop in the O.D. to less than 0.3 and the presence of stringy debris. At this point, 3.5 mls of 3M NaCl was added, the culture was transferred to a 50 ml tube and centrifuged at 8,000×G for 10 minutes to remove the debris. The supernatant was removed to a fresh tube and ⅕ volume of 50% polyethylene glycol (PEG) 8000 in water was added, mixed, and allowed to incubate at 4° C. overnight. The following morning, the PEG precipitate was spun down at 10,000 G for 20 minutes, and the pellet obtained after carefully removing all of the supernatant. The pellet was resuspended in 3 mls of TBS, split into two 2-ml plastic tubes, and spun in a microcentrifuge at maximum speed for 10 minutes to remove debris, and the supernatant collected. About ⅙ volume of 50% PEG was added to each tube for a second precipitation step and the mixture was incubated on ice for 60 minutes and then spun at maximum speed for 10 minutes in a microcentrifuge. The supernatant was discarded and the pellet resuspended in 300 μL of TBS. The resulting solution was spun again at maximum speed in a microcentrifuge for 10 minutes to remove debris. The resulting supernatant was titered and contained typically about 5×10¹¹ phage particles (pfu) per ml. This preparation was used for the following steps.

A negative selection step was then performed. The hu14.18 monoclonal antibody was bound to DYNAL beads through biotinylated goat anti-human antiserum as described above. 100 μL of the phage preparation produced as described in the preceding paragraph was adsorbed to the beads for 1 hour at room temperature in a solution of 1×Blocking Buffer. The beads were magnetically separated as described above, and the supernatant was withdrawn. This supernatant was then used to perform a second round of positive selection performed as described above, except that the phage-bead adsorbtion mixtures were washed 12 times for one minute each with TBS containing 0.1% Tween. The purpose of these changes was to increase the stringency of selection. The bound phages were eluted, expanded, and purified as described above. The resulting phage preparation was titered. The phage preparation was also used to perform another round of negative and positive selection using the same conditions described in the beginning of this paragraph, whose dual purposes were to serve as a backup in case the following steps failed, and to provide an indication of the trajectory of the selection. The number of phage that survived this third round of selection was significantly increased compared to the number of phage that survived the second round of selection, which suggests an enrichment of binding sequences.

The amplified phage population from the second round of selection was used to generate recombined proteins by the following procedure. Without wishing to be bound by theory, the rationale for this step was that the protein-target interactions of the initially selected phages might be due to only a subset of the loops in a given RD1Lib1 library member, and that tighter binding could be achieved by pairing such loops with a variety of loops in other positions, followed by selection of tight binders. This step is analogous to steps that naturally introduce diversity into antibody sequences.

About 1 μL of the phage preparation that had been eluted from the second selection and amplified, as well as 1 μL of the initial, unselected phage population, were used to initiate a PCR amplification of library member coding sequences. Each amplification reaction was cycled until a strong band appeared on a gel, representing about 10 ng/μL in the reaction. The reaction was then diluted 2-3 times with fresh PCR buffer, dNTPs, polymerase, and primers, and a single cycle performed to reduce the incidence of imperfectly paired library members.

The amplified products were purified with a Qiagen kit and cut with the restriction enzyme BstAPI, resulting in the production of four fragments: a 5′ and a 3′ fragment from the selected population, and a 5′ and a 3′ fragment from the unselected population. These were gel-purified according to standard procedures.

Three ligation reactions were then performed: 5′ selected plus 3′ selected; 5′ unselected plus 3′ selected; and 5′ selected plus 3′ unselected. Each ligation reaction was amplified. During the amplifications, samples were withdrawn at various times and quantitated on an agarose gel, from which it was verified that at least about 10⁹ independent and amplifiable ligated molecules had been created in each ligation reaction. The ligation reaction mixtures were purified with a Qiagen kit and simultaneously digested with EcoRI and HindIII. A 320-bp DNA fragment was gel purified and then ligated into T7Select 1-1b and packaged using a Novagen in vitro packaging kit in accordance with the manufacturer's instructions.

The new library was amplified and concentrated by the same protocol as was the original library, resulting in concentrated phage suspensions with titers of at least 5.0×10¹¹/ml. The selection procedures outlined above were used to select high affinity binders from the new library. In this instance, the third round of selection was not for backup but was the final round from which the best binders were to be screened.

### Example 5

### Testing Individual Phage-Based RD1Lib1 Library Members for Binding to Target Proteins

After a series of selections for phage-based binding proteins, the resulting population will generally contain a mixture of some phages that express a library member that binds to a target, and other phages that do not. To identify individual phages that express an RD1Lib1 library member capable of binding to a given target, ELISA-type plates were coated with a particular target molecule, clonal phages expressing a library member were added, and the extent of phage binding was detected using an antibody against a major phage capsid protein.

The following specific protocol was used in some cases. The wells of Nunc-Immuno Module MaxiSorp 8-Framed Immunoplates (catalogue CA#468667) were incubated with 100 μL of 1 μg/ml of a target protein overnight at 4° C. to coat the well with the target protein. The wells were washed four times with PBS plus 0.05% Tween-20. The wells were incubated for 2 hours at room temperature with 100 μL of PBS plus 3% bovine serum albumin to block, and again washed four times with PBS plus 0.05% Tween-20.

In parallel, clonal phages expressing a specific RD1Lib1 library member were generated as follows. The collection of phages from the selection in Example 4 were titered according to standard procedures. From an agar plate with well-separated plaques at least 1-2 mm in diameter, single plaques were picked as agar plugs using 200 μL widebore pipette tips and placed into the wells of a first Falcon Plastic 96-well U-bottom plate containing 50 μL of TE buffer (100 mM Tris/HCl pH8.0, 10 mM EDTA, pH 8.0) in each well. The plates were shaken on a tabletop shaker (Eppendorf) at room temperature for about 30 minutes to elute the phage particles. About 100 μL of exponentially growing E. coli strain 5403 (Novagen) at an O.D. of 0.5 at 600 nm was placed into the wells of a second 96-well U-bottom tissue culture plate and about 15 to 20 μL of eluted phage were added from the first 96-well plate. Two wells were left free of phage for use as controls so that lysis could be visually observed. The plate was covered with “breathable tape” and placed in a New Brunswick rotary shaker at about 900-1000 rotations per minute. The plate was visually monitored for lysis, which usually occurred after about 2 or more hours. About 20 μL of crude lysate from each well was then added to the wells of a Costar 3958 1 ml round-bottom plate, with each well containing 0.7 mls of exponentially growing E. coli strain 5403 at an O.D. of about 0.5. The plate was covered with breathable tape and placed in a New Brunswick rotary shaker at about 900-1000 rotations per minute. The plate was visually monitored for lysis, which usually occurred after about 2 or more hours.

For each 96-well plate of isolated phage clones, one 96-well ELISA plate was coated with target as described above, and one 96-well ELISA plate was coated with a non-target molecule to serve as a negative control. In the case of the anti-CD 19 antibody target, the second plate contained either chimeric KS antibody or chimeric 14.18 antibody. 100 μL of filtered phage were withdrawn from each well of the phage preparation, then 50 μL were added to the corresponding well on the target-coated ELISA plates and 50 μL added to a well on the negative control plate. The target and control plates were incubated for about 1 hour at room temperature. The plates were washed four times with PBS plus 0.05% Tween-20. About 100 μL of a 1:10,000 dilution of an anti-T7 tail protein monoclonal antibody (Novagen catalogue # 71530; Madison, Wis.) were added to each well, and incubation proceeded for about 1 hour at room temperature. The plates were washed four times with PBS plus 0.05% Tween-20. About 100 μL of Goat Anti-Mouse IgG, Fc HRP (Jackson Immuno catalogue # 115-035-071) at 1:10,000 were added, and incubation proceeded for about 1 hour at room temperature. The plates were washed four times with PBS plus 0.05% Tween-20. About 100 μL of Bio FX TMB Component HRP solution TMBW 1000-01 were added to each well for about 10-20 minutes, the reaction was terminated by addition of 100 μL of 1 N HCl, and the plates were read at 450 nm on a plate reading spectrophotometer.

### Example 6

### Testing Isolated RD1Lib1 Library Members for Binding to Target Proteins

As an alternative or following step to the characterization described in Example 5, the procedure described below was used to generate histidine-tagged library members derived from the phage-based library members generated in Example 4, but separated from the phage.

As a first step, a ‘mini-library’ was generated from the selected phage by PCR amplification of the RD1Lib1-encoding segments within the phage DNAs. The resulting DNA was cut with the enzymes NcoI and XhoI, and inserted into the pET30 vector (Novagen), such that an N-terminal histidine-tagged version of each RD1Lib1 library member would be expressed. Ligation reactions were performed according to standard conditions and Blri cells (Novagen) were transformed with the ligation reaction mix and plated on LB+50 mg/liter Kanamycin plates according to standard procedures.

Individual colonies were picked into round-bottom 96-well plates with 100 μL of 2x-NZCYM-Kan in each well, and grown overnight at 37 degrees C., shaking at about 900 RPM. The following day, the overnight cultures were diluted 1:50 or 1:100 in a new deep-well 96-well plate with 1 ml of 2xNZCYM +Kan, grown at 37° C. for several hours until a typical well showed an OD at 600 nm of 0.5, induced with 0.5 mM IPTG and then allowed to grow at 37° C. for an additional 4 hours. This step results in the cytoplasmic expression of individual histidine-tagged library members. The cultures were then lysed using either “Bug Buster” or “Pop Culture” (both Novagen), according to the instructions of the manufacturer. After the centrifugation step that removes cell debris following lysis, the supernatant was moved to a fresh plate. This supernatant contained the soluble RD1Lib1 proteins. Random wells were selected for PAGE, to ensure that expression was adequate in at least a significant number of the clones. The original overnight cultures were retained, either as glycerol stocks at minus 80° C., or as a replica on an LB-Kan plate, for future sequencing or further testing.

The binding properties of the various clones was tested as follows. For each 96 well plate of clones, two 96 well Nickel-NTA plates (Pierce) were prepared, one to be an experimental and the other a control. 80 μL of binding buffer (300 mM NaCl, 25 mM sodium phosphate, pH 8.0) was added to each well in both plates, then 20 μL of supernatant from the RD1 preparation was added to the two plates, in the same position as in the original prep. The lysate was well mixed with the binding buffer, then allowed to incubate for one hour, in order to as fully as possible saturate the Nickel-NTA sites on the plate bottom. The plates were then washed 4 times with TBS plus 0.05% Tween (TBS-T). Two solutions were prepared in TBS, one with the target (anti-CD19) at 2 μg/ml, the other with the negative control (14.18) also at 2 μg/ml. 100 μL of the target solution was added to each well of the experimental plates, and 100 μL of the control solution added to each well of the control plates. After one hour the plates were washed 4× with TBS-T, then goat anti-human IgG (Fc) antibody conjugated to HRP (Jackson Immunolaboratories) at a 1:10,000 dilution in TBS was added and incubated for 1 hour. The plates were then washed 4× in TBS-T, and the signal developed by the addition of 100 μL/well of Bio-FX TMB as described in Example 5.

About 50% of the tested RDILibI library members appeared to bind to the preselected anti-CD19 target molecule. In this case, the library members were also tested for binding to the 14.18 antibody. Only one of the selected library members appeared also to bind 14.18. This library member most likely binds to a constant region of the antibodies, and thus appears to represent an escape from the negative selection steps described in Example 4.

Taken together, these results confirm that RDILibI library members can be identified that bind to a preselected target molecule in a specfic manner.

### Example 7

### Binding Proteins to Anti-αV Antibody Variable Domain

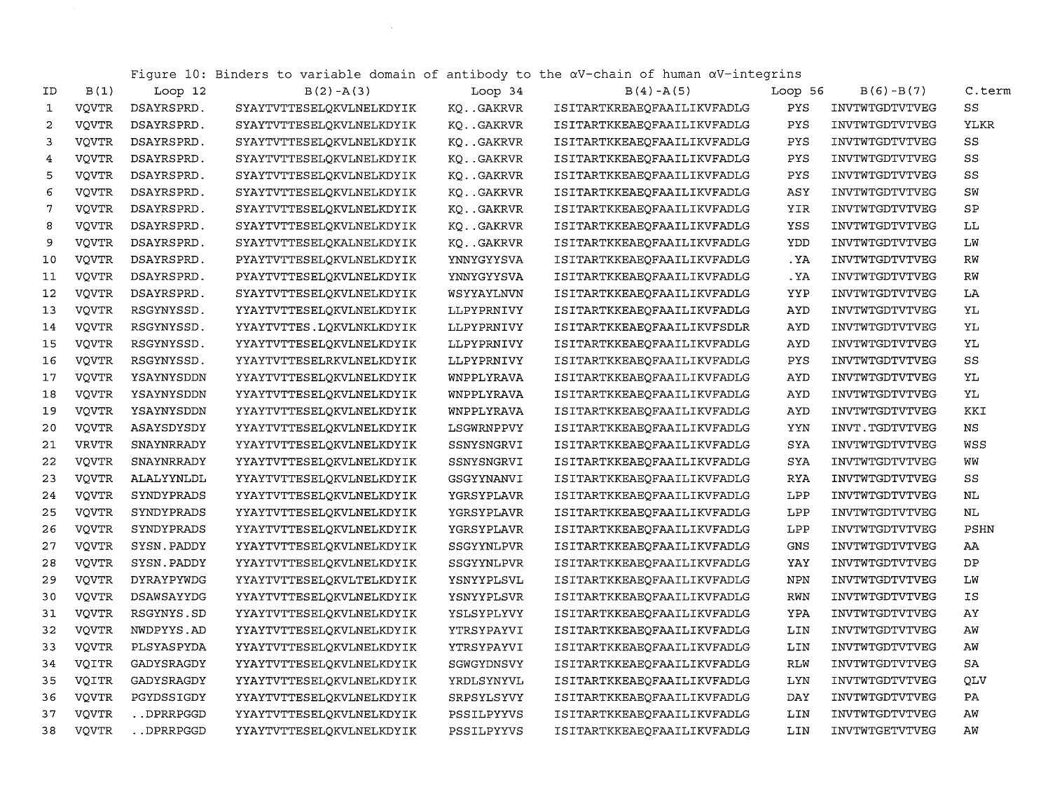

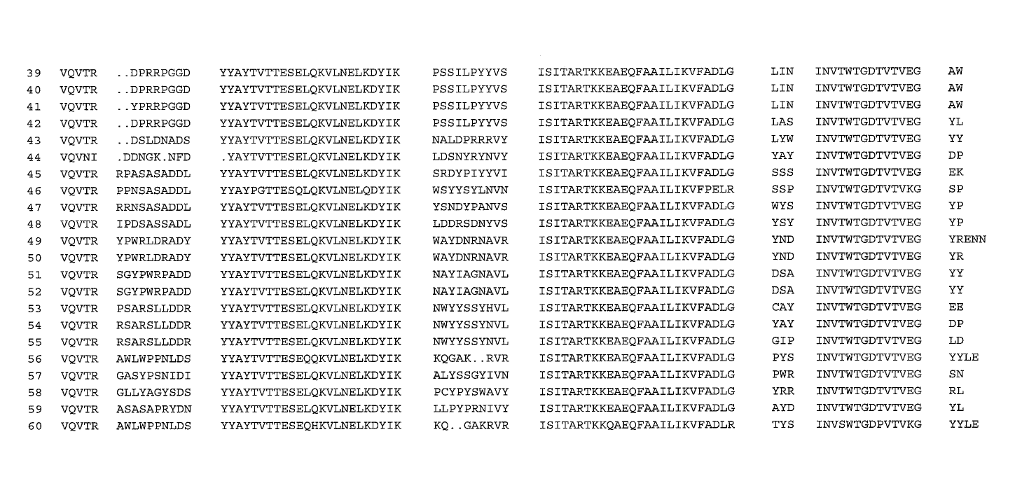

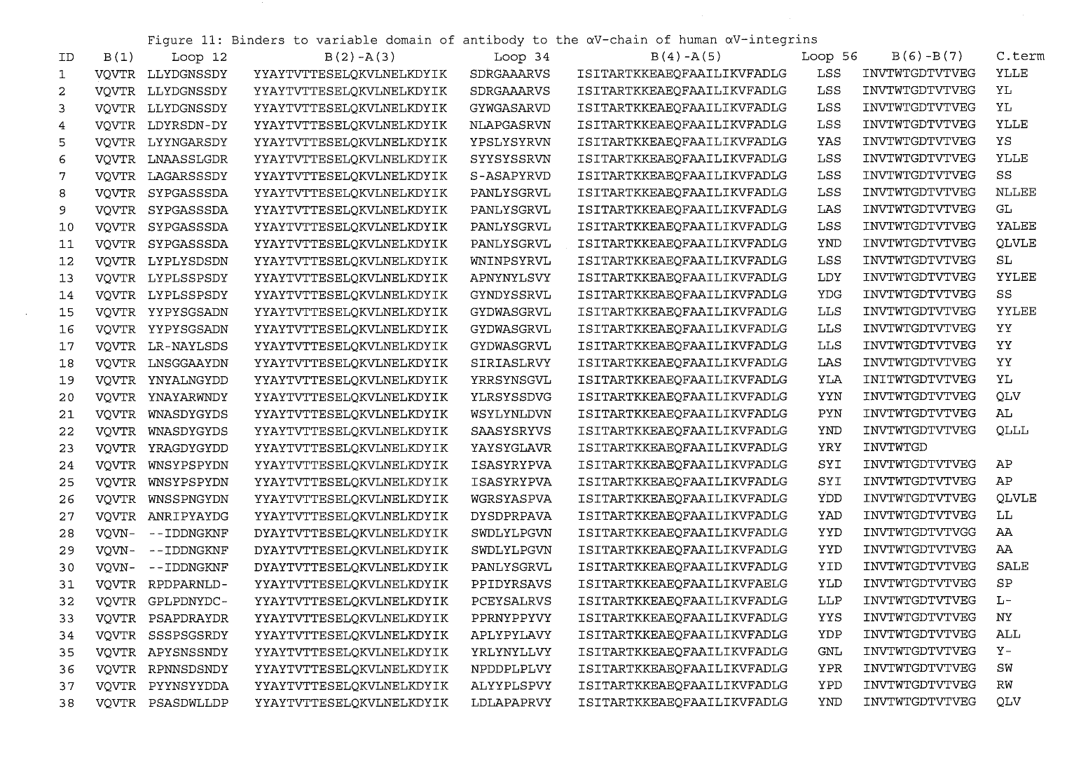

An RD1Lib1 library was successfully screened for proteins with an affinity for the variable domain of an antibody to the αV-chain of human αV-integrins (see U.S. Pat. No. 5,985,278). The amino acid sequences of the identified proteins are presented in FIGS. 10 and 11.

### Example 8

### Binding Proteins to KS

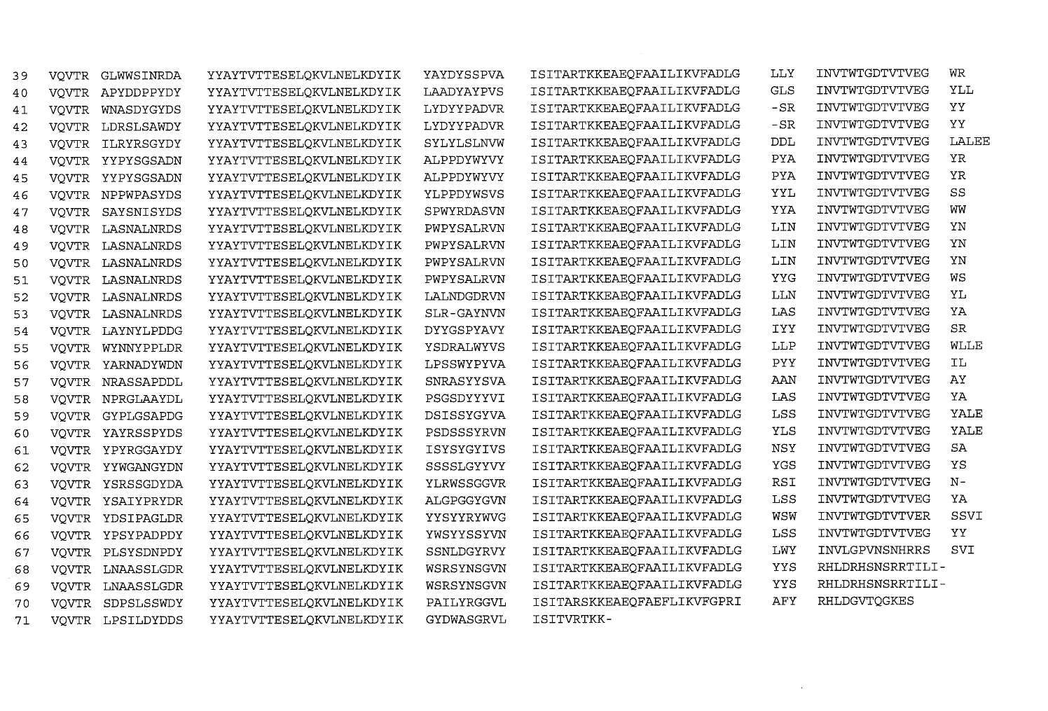

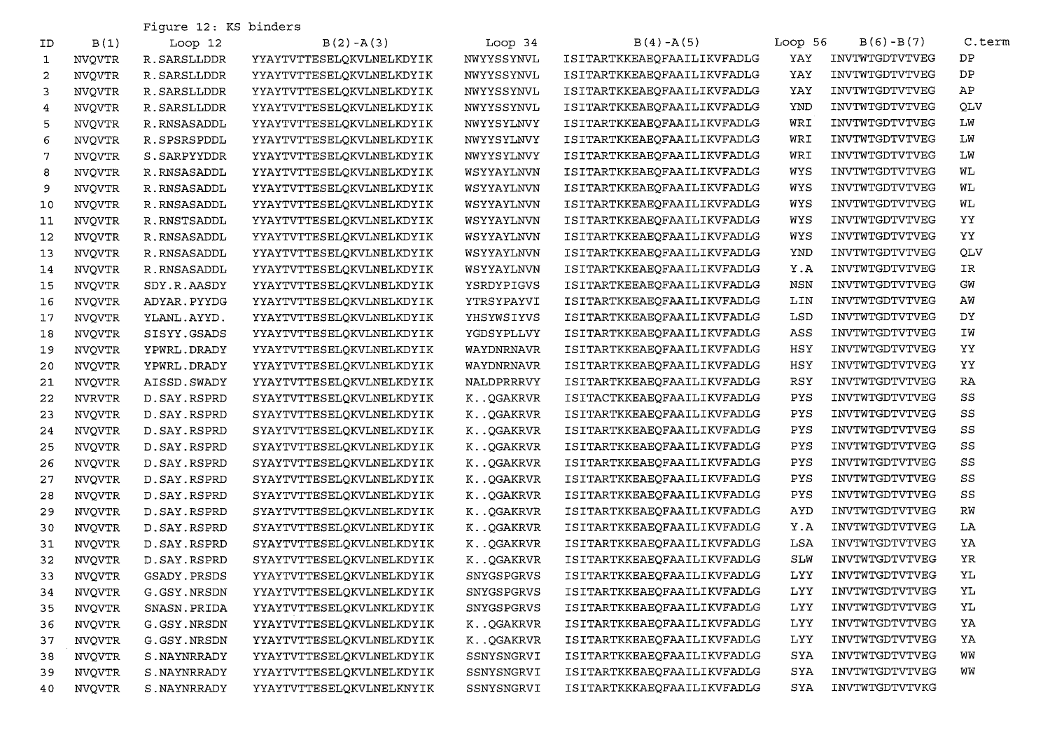

An RD1 Lib1 library was successfully screened for proteins with an affinity for a humanized KS antibody variable domain, which recognizes the human EpCAM antigen. The amino acid sequences of the identified proteins are presented in FIG. 12.

### Example 9

### Binding Proteins to Anti-CD19 Antibody Variable Domain, and to IgG

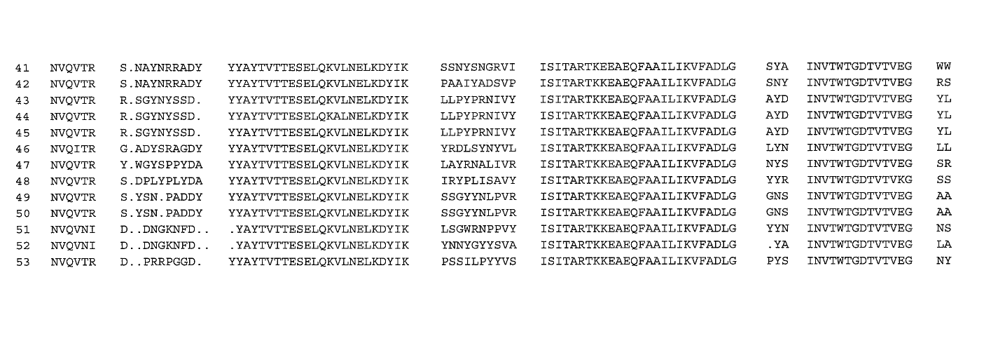

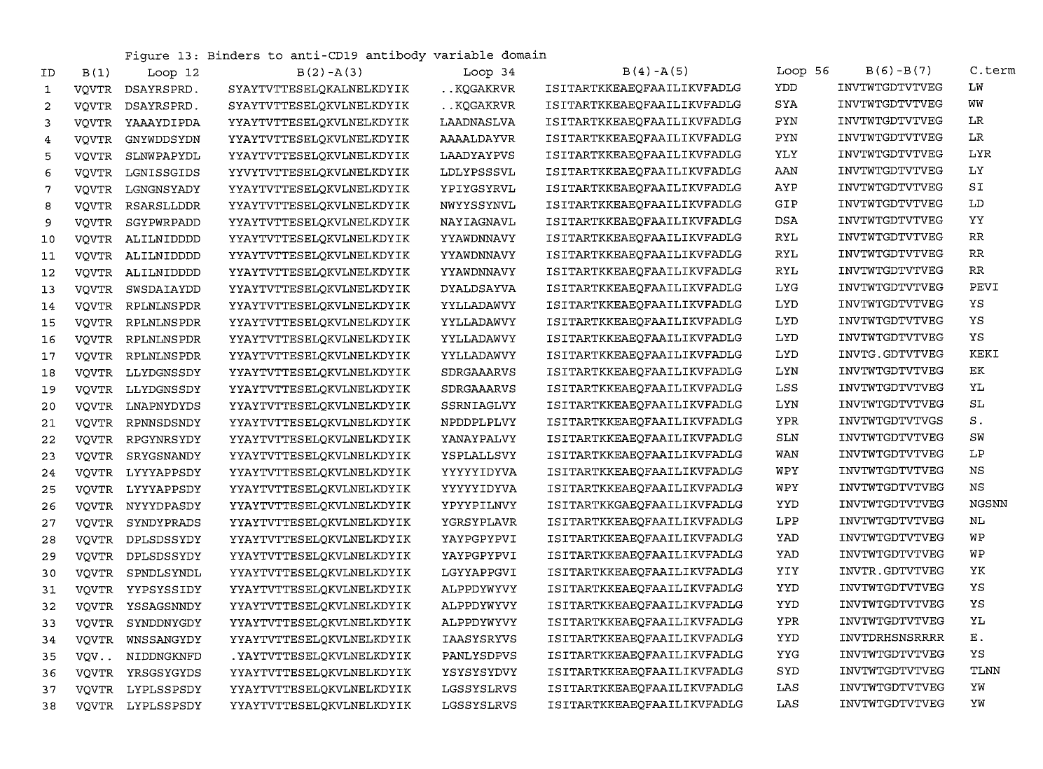

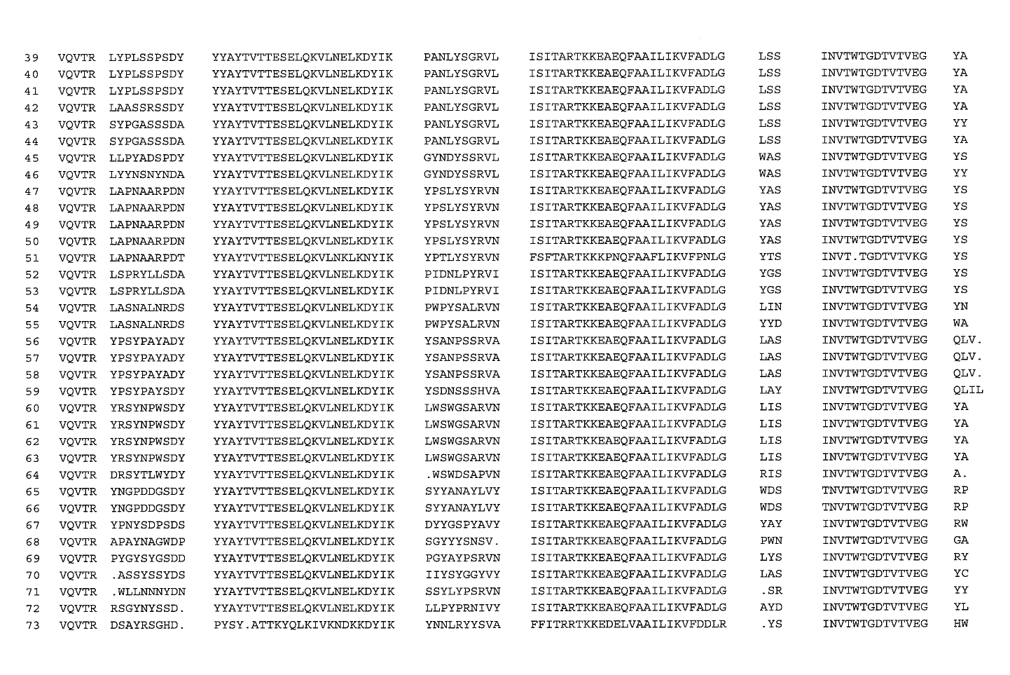

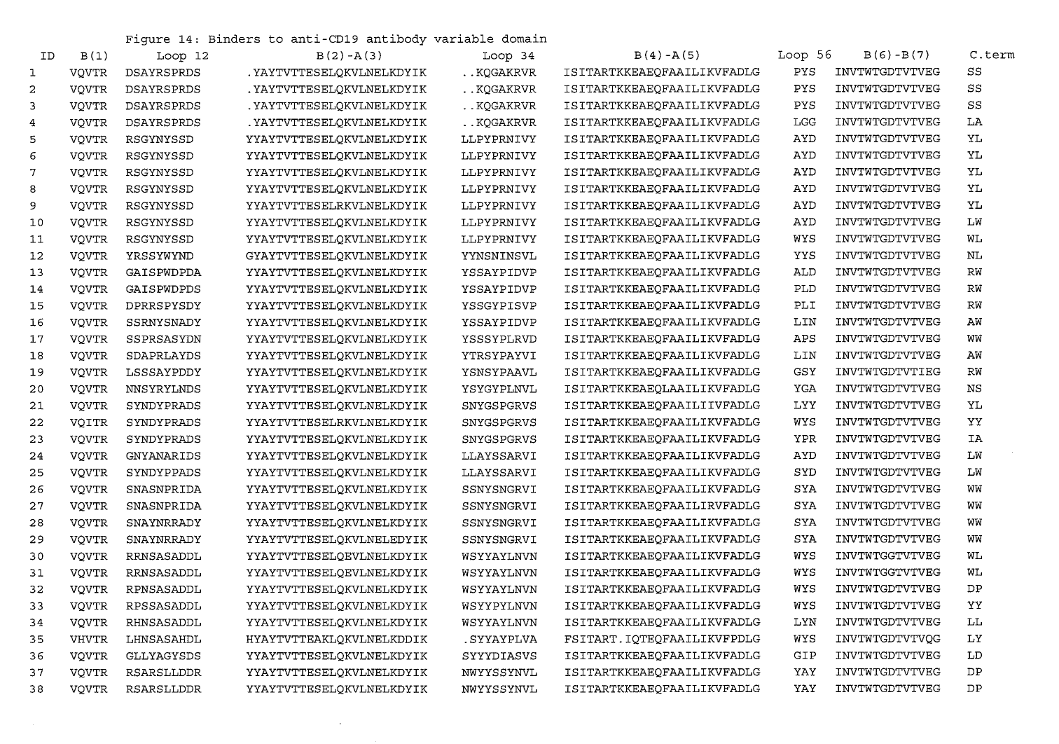

An RD1Lib1 library was successfully screened for proteins with an affinity for the variable domain of an anti-CD19 antibody. The amino acid sequences of the identified proteins are presented in FIGS. 13 and 14.

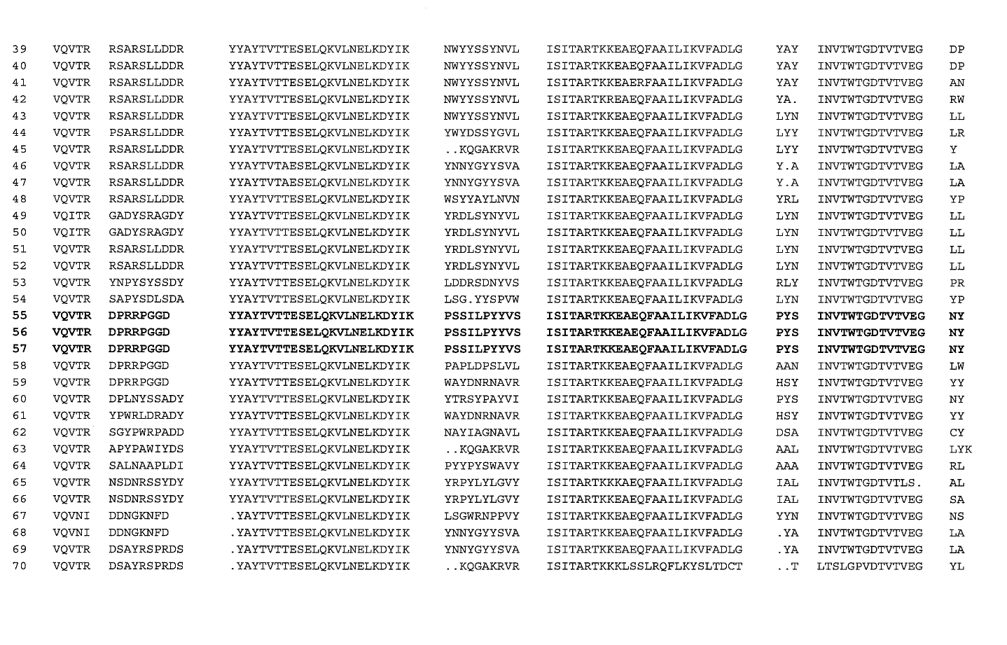

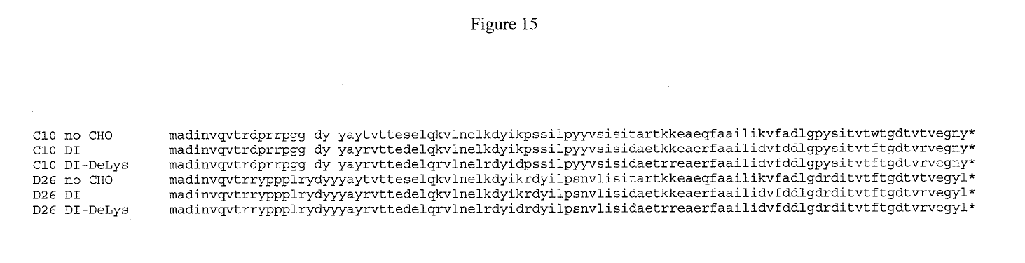

One anti-CD19 antibody binding protein, designated CIO, was selected for additional protein design work. Specifically, additional proteins were designed in which the randomized sequences of C10 were grafted into alternative scaffold sequences. The first such scaffold, designated “RD1 no CHO” or simply “no CHO,” is a version of RD1.3 with a mutated glycosylation site. The second scaffold, designated “DI,” is a deimmunized version of RD1.3. The third scaffold, designated “DI-DeLys,” is a version of DI in which each lysine has been replaced with an arginine. An IgG binding protein, designated D26, was also selected for grafting of its randomized sequences into these other scaffolds. The resulting amino acid sequences are depicted in FIG. 15.

### Example 10

### Confirmation of Non-Aggregation of RD1 Variants and Fc Fusion Proteins

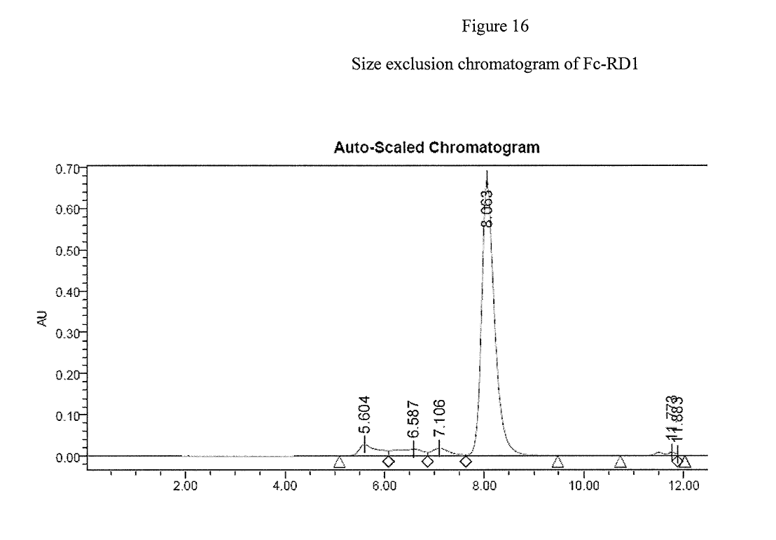

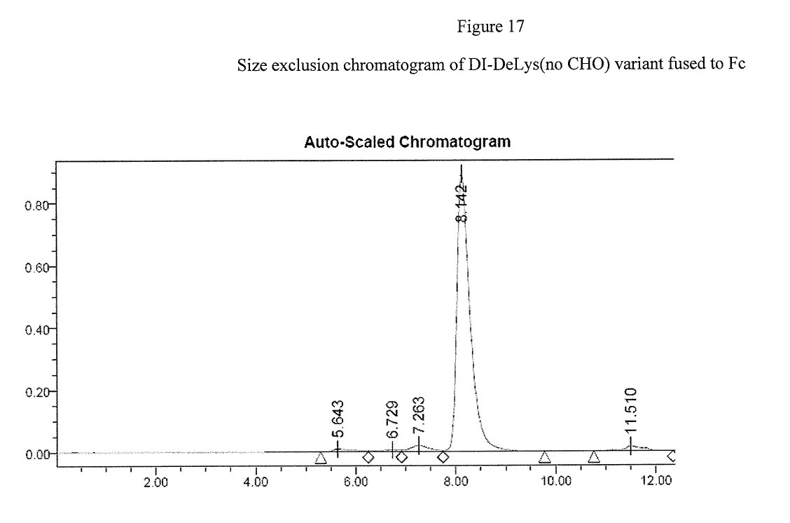

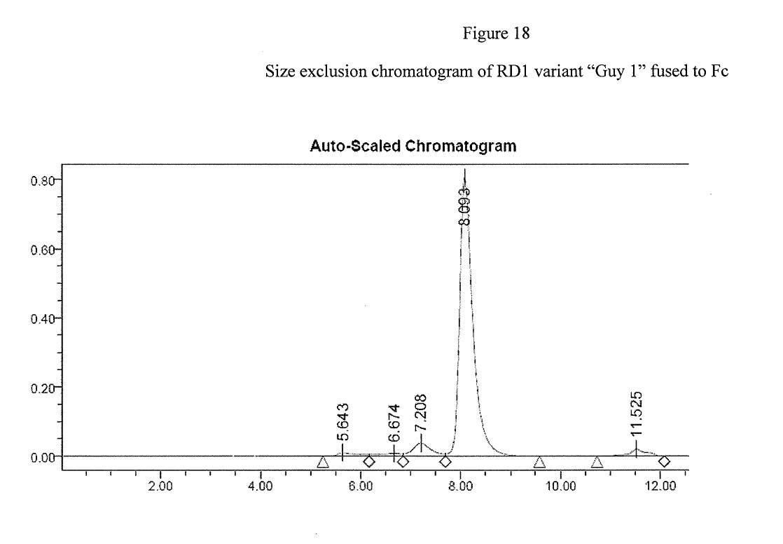

Three scaffold proteins were subjected to size exclusion chromatography to confirm that the proteins were present primarily as non-aggregated monomers. These included a fusion protein with an Fc antibody fragment at the N-terminus of the fusion protein and RD 1.3 at the C-terminus; RD1-DI-DeLys; and RD1 variant “Guy 1” from FIG. 9. The size exclusion chromatograms for Fc-RD1, RD1-DI-DeLys, and Guy 1 are shown in FIGS. 16, 17 and 18, respectively. As can be seen in the Figures, each protein is present primarily as a single peak in the chromatograms, indicating that the protein is present in a non-aggregated form.

### Example 11

### Synthesis of Additional Variants

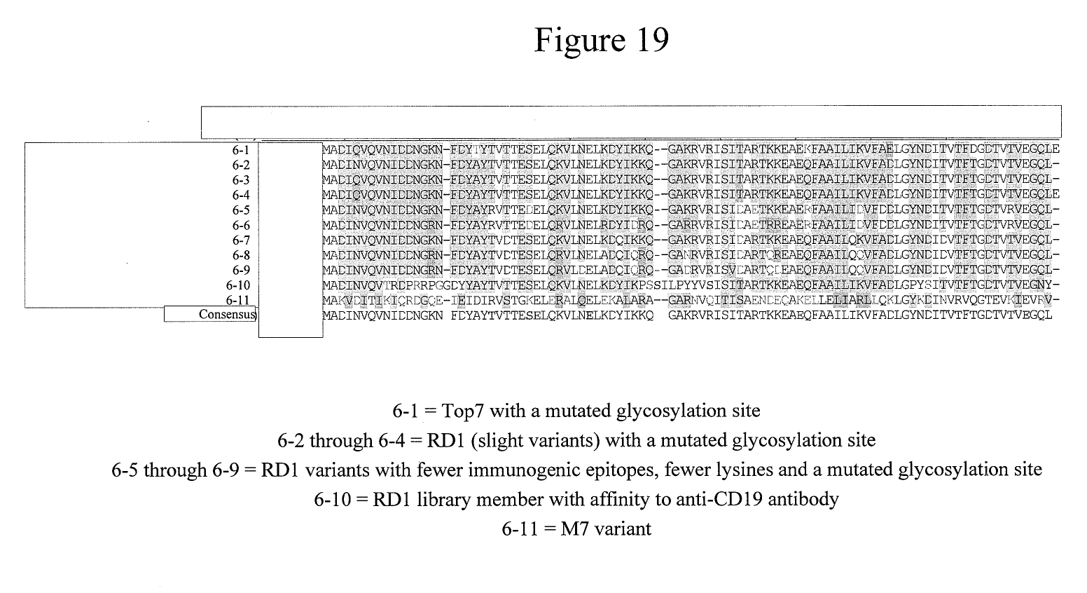

Additional variant scaffold proteins were designed and synthesized. The sequences of these proteins are depicted in FIG. 19. These include: 6-1, a Top7 protein with a mutated glycosylation site; 6-2 through 6-4, slight variants of RD1.3, 6-5 through 6-9, RD1.3 variants with fewer immunogenic epitopes and fewer lysines; 6-10=an RD1 library member from Example 9; and 6-11, a variant on the M7 protein of Dallüge et al. All of these proteins were successfully expressed, as determined by subsequent denaturing and non-denaturing gel electrophoresis.

### INCORPORATION BY REFERENCE

The entire disclosure of each of the patent documents and scientific articles referred to herein is incorporated by reference for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A protein comprising a Top7 fold, wherein one or more loops in the Top7 fold bind specifically to a preselected target molecule, wherein the protein binds to the preselected target molecule with a dissocation constant of no more than 10 μM.

2. A protein comprising a Top7 fold that defines two ends, wherein at least two loops on one end of the protein are each at least one amino acid longer than the corresponding loops of Top7.

3-23. (canceled)

24. A protein comprising an amino acid sequence of the formula B(1)-L(12)-B(2)-L(23)-A(3)-L(34)-B(4)-L(45)-A(5)-L(56)-B(6)-L(67)-B(7),
wherein B(1), B(2), A(3), B(4), A(5), B(6), and B(7) correspond to amino acids 1-5, 6-8, 9-20, 21-23, 24-32, 33-37, and 38-42 of (i) an amino acid sequence at least 85% identical to SEQ ID NO:3; or (ii) a sequence at least 95% identical to SEQ ID NO:6; or (iii) a sequence identical to SEQ ID NO:7,
wherein the minimum length of L(12) is 10 amino acids,
wherein the minimum length of L(23) is 7 amino acids,
wherein the minimum length of L(34) is 9 amino acids,
wherein the minimum length of L(45) is 10 amino acids,
wherein the minimum length of L(56) is 7 amino acids,
wherein the minimum length of L(67) is 4 amino acids, and
wherein L(12), L(23), L(34), L(45), L(56), or L(67) exceeds its minimum length by at least one amino acid.

25-37. (canceled)

38. A protein according to claim 1, wherein the protein does not specifically bind CD4.

39. A protein according to claim 1, wherein the protein does not comprise a human immunodeficiency virus peptide.

40. A protein according to claim 1, wherein the protein does not comprise an immunogenic human immunodeficiency virus peptide.

41. A protein according to claim 1, wherein the protein does not comprise a viral peptide.

42. A protein according to claim 1, wherein the protein does not comprise a bacterial peptide.

43. A fusion protein comprising at least two proteins according to claim 1.

44. A protein library comprising a plurality of non-identical proteins each according to claim 2, wherein the non-identical proteins differ from each other in the amino acid sequences of one or more of the loops.

45. (canceled)

46. (canceled)

47. A nucleic acid library encoding a protein library according to claim 44.

48. A nucleic acid encoding a protein according to claim 1.

49. A cell comprising the nucleic acid of claim 48.

50. A complex comprising:
a protein according to claim 1, and
the preselected target molecule.

51. The complex of claim 50, further comprising a detectable label.

52. A method of identifying a protein that specifically binds a preselected target molecule, the method comprising:
exposing a protein library according to claim 44 to a target molecule; and
identifying at least one protein associated with the target molecule.

53. A method for detecting a target molecule, the method comprising:
exposing a sample to a protein according to claim 1 under conditions permitting a target molecule, if present, to bind to the protein; and
detecting the presence or absence of a complex comprising the protein and the target molecule.

54. A method of binding to an in vivo target, the method comprising administering a protein according to claim 1, wherein the protein specifically binds an in vivo target.

55. The method of claim 54, wherein the protein further comprises a detectable label.

56. The method of claim 54, wherein the protein further comprises an effector stably associated therewith.
